(19) United States
(12) Patent Application Publication
(10) Pub. No.: US 2021/0382280 A1
(43) Pub. Date: Dec. 9, 2021

(54) IMAGING LENS AND IMAGING APPARATUS

(71) Applicant: SONY CORPORATION, Minato-ku (JP)

(72) Inventors: YOSHIO HOSONO, TOKYO (JP); YASUHIDE NIHEI, TOKYO (JP); DAIGO KATSURAGI, TOKYO (JP); ISAO YAMAMOTO, TOKYO (JP)

(21) Appl. No.: 17/287,239

(22) PCT Filed: Oct. 8, 2019
(86) PCT No.: PCT/JP2019/039567

§ 371 Date: Apr. 21, 2021

(30) Foreign Application Priority Data
Oct. 29, 2018 (JP) 2018-203211

(51) Int. Cl. G02B 13/00; G02B 9/64

(52) U.S. Cl. CPC G02B 13/0045; B60R 16/0232; G02B 9/64

(57) ABSTRACT

An imaging lens of the present disclosure includes, in order from object side toward image plane side: a first lens having positive refractive power in the vicinity of an optical axis; a second lens having positive refractive power in the vicinity of the optical axis; a third lens having negative refractive power in the vicinity of the optical axis; a fourth lens having negative refractive power in the vicinity of the optical axis; a fifth lens having negative refractive power in the vicinity of the optical axis; a sixth lens having negative refractive power in the vicinity of the optical axis; and a seventh lens of which a lens surface on the image plane side has an aspheric shape having an inflection point.

### TECHNICAL FIELD

The present disclosure relates to an imaging lens that forms an optical image of an object on an imaging element such as CCD (Charge Coupled Device) and CMOS (Complementary Metal Oxide Semiconductor), and an imaging apparatus mounted with the imaging lens to perform photographing, e.g., a digital still camera, a mobile phone with a camera, and an information mobile terminal.

### BACKGROUND ART

For digital still cameras, low-profile ones such as a card type are produced year by year, and size reduction in imaging apparatuses has been desired. Moreover, for mobile phones as well, the size reduction in imaging apparatuses has been desired, to provide a low-profile terminal itself or to provide sufficient space for mounting many functions. Thus, there has been an increasing desire for further size reduction in imaging lenses to be mounted on imaging apparatuses.

Moreover, simultaneously with the size reduction in imaging elements such as CCD and CMOS, there has been advancement in an increase in the number of pixels, by miniaturization of a pixel pitch of the imaging elements. In accompaniment therewith, higher performance has been desired for the imaging lenses to be used in such imaging apparatuses.

### CITATION LIST

### Patent Literature

PTL 1: Japanese Unexamined Patent Application Publication No. 2015-55728
PTL 2: Japanese Unexamined Patent Application Publication No. 2015-203792
PTL 3: Japanese Unexamined Patent Application Publication No. 2016-71115

### SUMMARY OF THE INVENTION

Furthermore, there has been a desire for a great-aperture and bright lens that allows for high sensitivity shooting while preventing image quality from lowering because of noises in shooting in dark places.

It is desirable to provide a high-performance imaging lens that attains size reduction and an increase in aperture size, and an imaging apparatus mounted with such an imaging lens.

An imaging lens according to one embodiment of the present disclosure includes, in order from object side toward image plane side: a first lens having positive refractive power in the vicinity of an optical axis; a second lens having positive refractive power in the vicinity of the optical axis; a third lens having negative refractive power in the vicinity of the optical axis; a fourth lens having negative refractive power in the vicinity of the optical axis; a fifth lens having negative refractive power in the vicinity of the optical axis; a sixth lens having negative refractive power in the vicinity of the optical axis; and a seventh lens of which a lens surface on the image plane side has an aspheric shape having an inflection point.

An imaging apparatus according to one embodiment of the present disclosure includes an imaging lens and an imaging element that outputs an imaging signal corresponding to an optical image formed by the imaging lens. The imaging lens includes the imaging lens according to the embodiment of the present disclosure described above.

In the imaging lens or the imaging apparatus according to the embodiment of the disclosure, with a seven-lens overall configuration, optimization of a configuration of each lens is attained.

### BRIEF DESCRIPTION OF THE DRAWINGS

FIG. 1 is a lens cross-sectional view of a first configuration example of an imaging lens according to an embodiment of the present disclosure.

FIG. 2 is a lens cross-sectional view of a second configuration example of the imaging lens.

FIG. 3 is a lens cross-sectional view of a third configuration example of the imaging lens.

FIG. 4 is a lens cross-sectional view of a fourth configuration example of the imaging lens.

FIG. 5 is a lens cross-sectional view of a fifth configuration example of the imaging lens.

FIG. 6 is a lens cross-sectional view of a sixth configuration example of the imaging lens.

FIG. 7 is a lens cross-sectional view of a seventh configuration example of the imaging lens.

FIG. 8 is a lens cross-sectional view of an eighth configuration example of the imaging lens.

FIG. 9 is a lens cross-sectional view of a ninth configuration example of the imaging lens.

FIG. 10 is an aberration diagram illustrating various aberrations in Numerical example 1 to which specific numerical values are applied to the imaging lens illustrated in FIG. 1.

FIG. 11 is an aberration diagram illustrating various aberrations in Numerical example 2 to which specific numerical values are applied to the imaging lens illustrated in FIG. 2.

FIG. 12 is an aberration diagram illustrating various aberrations in Numerical example 3 to which specific numerical values are applied to the imaging lens illustrated in FIG. 3.

FIG. 13 is an aberration diagram illustrating various aberrations in Numerical example 4 to which specific numerical values are applied to the imaging lens illustrated in FIG. 4.

FIG. 14 is an aberration diagram illustrating various aberrations in Numerical example 5 to which specific numerical values are applied to the imaging lens illustrated in FIG. 5.

FIG. 15 is an aberration diagram illustrating various aberrations in Numerical example 6 to which specific numerical values are applied to the imaging lens illustrated in FIG. 6.

FIG. 16 is an aberration diagram illustrating various aberrations in Numerical example 7 to which specific numerical values are applied to the imaging lens illustrated in FIG. 7.

FIG. 17 is an aberration diagram illustrating various aberrations in Numerical example 8 to which specific numerical values are applied to the imaging lens illustrated in FIG. 8.

FIG. 18 is an aberration diagram illustrating various aberrations in Numerical example 9 to which specific numerical values are applied to the imaging lens illustrated in FIG. 9.

FIG. 19 is a front view illustrating a configuration example of an imaging apparatus.

FIG. 20 is a rear view illustrating the configuration example of the imaging apparatus.

FIG. 21 is a block diagram depicting an example of schematic configuration of a vehicle control system.

FIG. 22 is a diagram of assistance in explaining an example of installation positions of an outside-vehicle information detecting section and an imaging section.

FIG. 23 is a view depicting an example of a schematic configuration of an endoscopic surgery system.

FIG. 24 is a block diagram depicting an example of a functional configuration of a camera head and a camera control unit (CCU) depicted in FIG. 23.

### MODES FOR CARRYING OUT THE INVENTION

Some embodiments of the present disclosure are described below in detail with reference to the drawings. It is to be noted that the description is given in the following order.

0. Comparative Examples

1. Basic Configuration of Lens

2. Workings and Effects

3. Example of Application to Imaging Apparatus

4. Numerical Examples of Lens

5. Applied Examples
5.1 First Applied Example
5.2 Second Applied Example

6. Other Embodiments

### 0. Comparative Examples

In an imaging lens, it is conceivable to have a seven or more lens configuration, to attain size reduction and high performance. For example, PTL 1 (Japanese Unexamined Patent Application Publication No. 2015-55728), PTL 2 (Japanese Unexamined Patent Application Publication No. 2015-203792), and PTL 3 (Japanese Unexamined Patent Application Publication No. 2016-71115) disclose imaging lenses having a seven-lens configuration.

PTL 1 discloses an imaging lens including, in order from object side toward image plane side: a first lens having positive refractive power; a second lens having positive or negative refractive power; a third lens having negative refractive power; a fourth lens having positive or negative refractive power; a fifth lens having positive or negative refractive power; a sixth lens having positive or negative refractive power; and a seventh lens having negative refractive power. In PTL 1, as an example, no disclosure or suggestion has been made of a configuration in which all of the third lens, the fourth lens, the fifth lens, the sixth lens, and the seventh lens have negative refractive power. In the imaging lens described in PTL 1, positive refractive power to be concentrated on the first lens and the second lens is dispersed to the lenses other than the first lens and the second lens for the size reduction. Therefore, it has a disadvantageous configuration for the size reduction. Further, in the imaging lens described in PTL 1, a configuration in which a composite focal length of the fourth lens and the fifth lens is positive, and the fifth lens is a strong (large curvature) meniscus shape with a convex surface directed toward the image plane side becomes a factor that increases a thickness of the lens itself. Thus, it becomes disadvantageous in the size reduction. Furthermore, in the configuration of the imaging lens described in PTL 1, on the occasion of an increase in aperture size, there occurs possibility of insufficient correction on a spherical aberration generated on front side. This sometimes causes insufficient performance to suppress various aberrations while satisfying predetermined optical performance. Reviewing the power of each lens provides room for improvement.

PTL 2 discloses an imaging lens of a seven-lens configuration including: a first lens group including a first lens, a second lens, and a third lens; a second lens group including a fourth lens and a fifth lens; and a third lens group including a sixth lens and a seventh lens. In the configuration of the imaging lens described in PTL 2, to achieve the size reduction, it is desirable to increase positive refractive power of the first lens group, which means that a light beam is strongly refracted in the first lens group. In the imaging lens described in PTL 2, both the first lens group and the second lens group have positive refractive power. However, in the case of this configuration, allowing the light beam to refract strongly in the first lens group for the size reduction causes further refraction of the light beam in the second lens group. This results in further deterioration of the aberration, inhibiting an optimum correction on the aberration. Thus, reviewing the refractive power of the second lens group provides room for improvement.

PTL 3 discloses an imaging lens including first to seventh optical elements. The imaging lens described in PTL 3 has a feature that a single aberration correction optical element is disposed as the seventh optical element. The aberration correction optical element has substantially no refractive power, and both surfaces are aspheric. This configuration is unsuitable for the size reduction because of the seventh optical element having substantially no refractive power in the paraxial vicinity, and because of space to be involved in disposing the seventh optical element. Moreover, for aberration correction as well, although a correction effect outside the paraxial vicinity, there is no effect of aberration correction in the paraxial vicinity. It is therefore difficult to ensure predetermined optical performance, as compared to an imaging lens of a seven-lens configuration in which the seventh lens has refractive power. Thus, reviewing the configuration of the lens provides room for improvement.

Therefore, it is desirable to provide a high-performance imaging lens of a seven-lens configuration that attains the size reduction and an increase in aperture, and an imaging apparatus mounted with such an imaging lens of the seven-lens configuration.

### 1. BASIC CONFIGURATION OF LENS

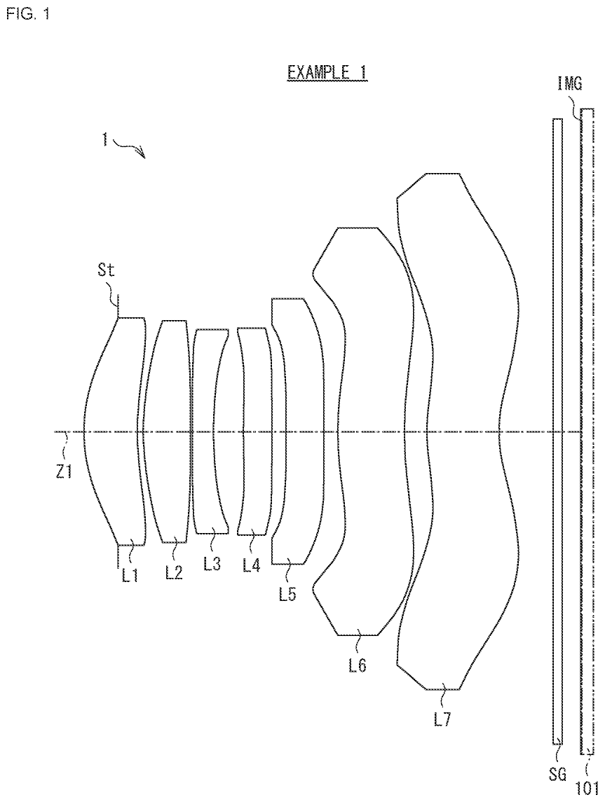

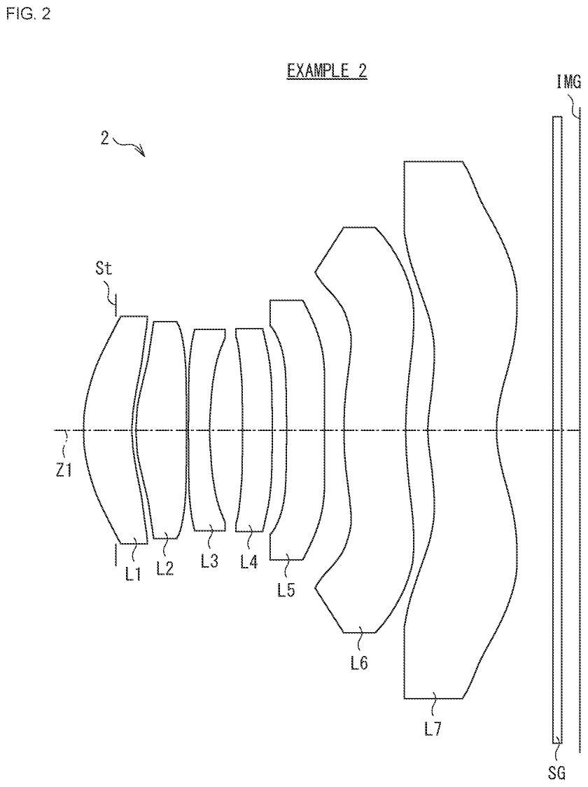

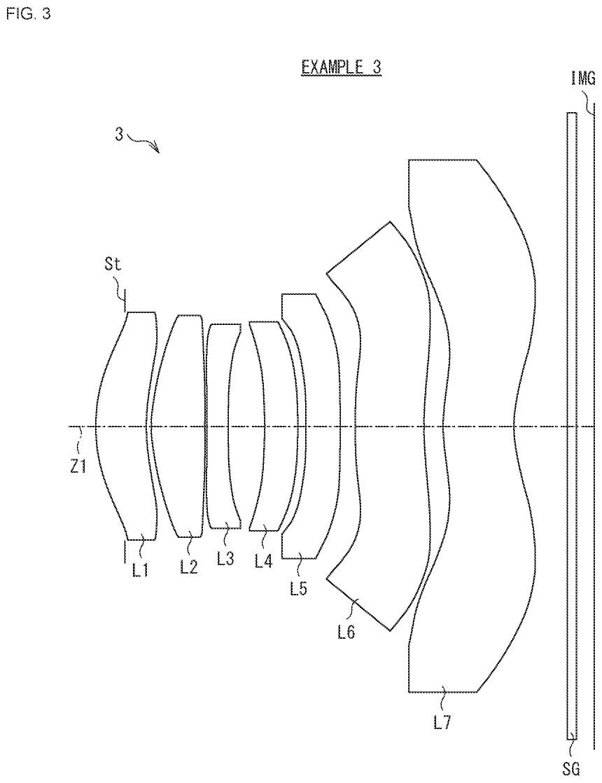

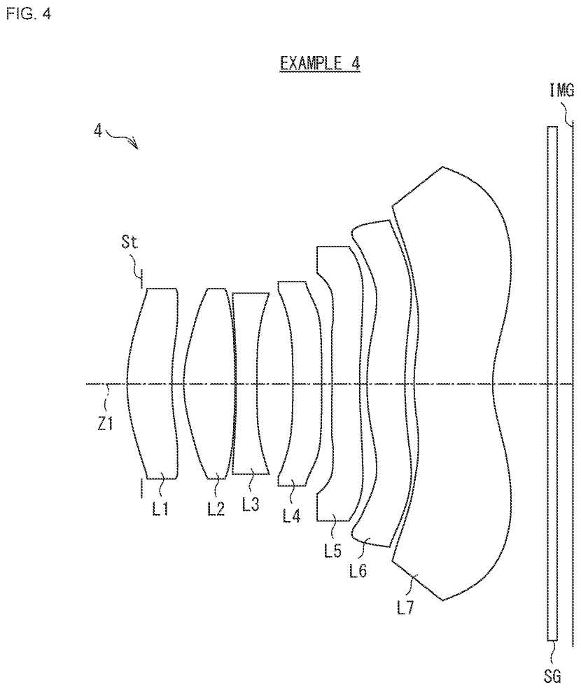

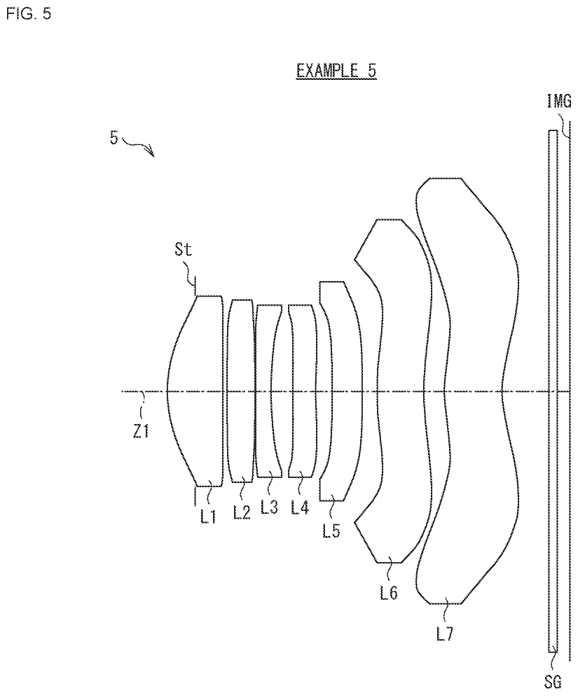

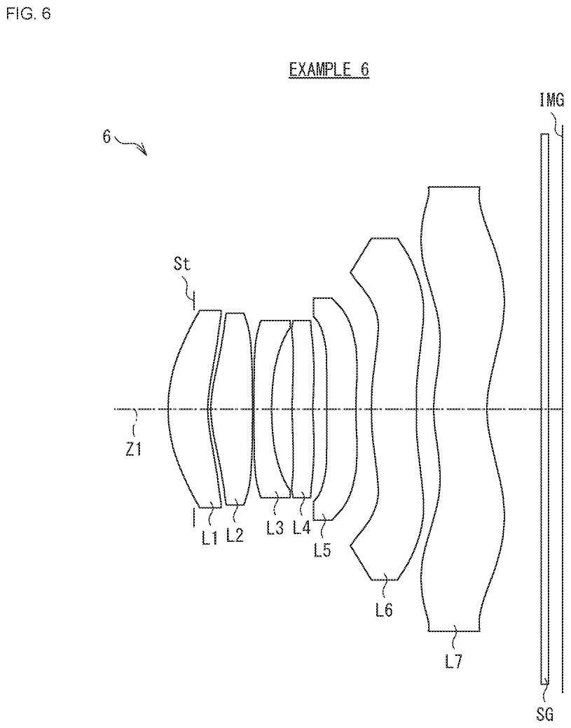

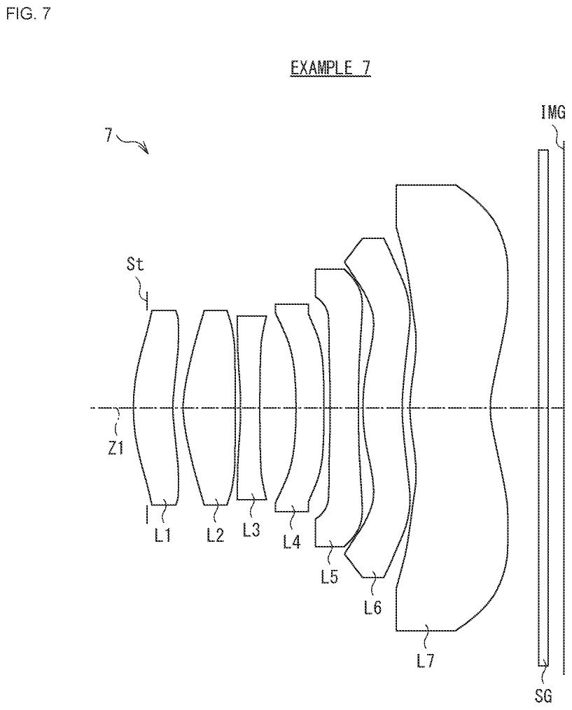

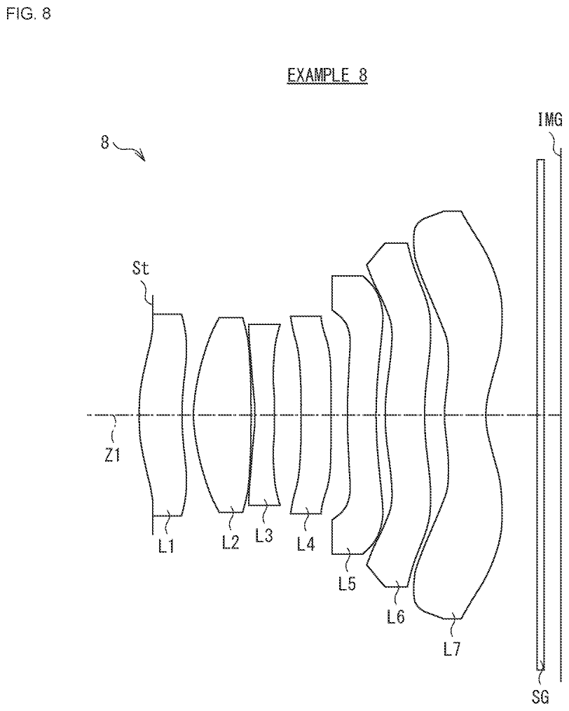

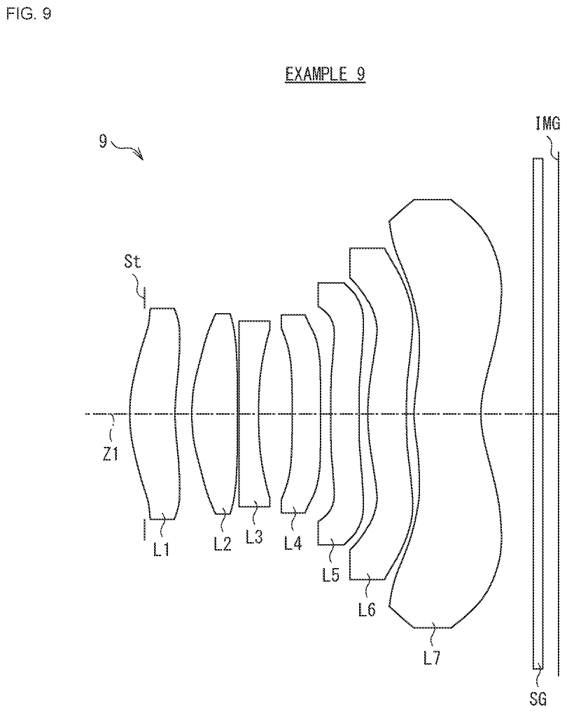

FIG. 1 illustrates a first configuration example of an imaging lens according to an embodiment of the present disclosure. FIG. 2 illustrates a second configuration example of the imaging lens. FIG. 3 illustrates a third configuration example of the imaging lens. FIG. 4 illustrates a fourth configuration example of the imaging lens. FIG. 5 illustrates a fifth configuration example of the imaging lens. FIG. 6 illustrates a sixth configuration example of the imaging lens. FIG. 7 illustrates a seventh configuration example of the imaging lens. FIG. 8 illustrates an eighth configuration example of the imaging lens. FIG. 9 illustrates a ninth configuration example of the imaging lens. Numerical examples in which specific numerical values are applied to these configuration examples are described later.

In FIG. 1 and the like, reference characters IMG represent an image plane, and Z1 represent an optical axis. St represent an aperture stop. In the vicinity of the image plane IMG, an imaging element 101 such as CCD and CMOS may be disposed. Between the imaging lens and the image plane IMG, an optical member such as a sealing glass SG protecting the imaging element and various optical filters may be disposed.

A configuration of the imaging lens according to the present embodiment is described below in association with the configuration examples illustrated in FIG. 1 and the like where appropriate. The technology of the present disclosure is, however, not limited to the illustrated configuration examples.

The imaging lens according to the present embodiment includes substantially seven lenses in which a first lens L1, a second lens L2, a third lens L3, a fourth lens L4, a fifth lens L5, a sixth lens L6, and a seventh lens L7 are disposed in order from object side toward image plane side along the optical axis Z1.

The first lens L1 has positive refractive power in the vicinity of the optical axis.

The second lens L2 has positive refractive power in the vicinity of the optical axis.

The third lens L3 has negative refractive power in the vicinity of the optical axis.

The fourth lens L4 has negative refractive power in the vicinity of the optical axis.

The fifth lens L5 has negative refractive power in the vicinity of the optical axis.

The sixth lens L6 has negative refractive power in the vicinity of the optical axis.

The seventh lens L7 has positive or negative refractive power in the vicinity of the optical axis. It is desirable that in the seventh lens L7, a lens surface on the image plane side have an aspheric shape having an inflection point.

In addition, it is desirable that the imaging lens according to the present embodiment further satisfy predetermined conditional expressions and the like which are described later.

### 2. WORKINGS AND EFFECTS

Next, workings and effects of the imaging lens according to the present embodiment are described. A more desirable configuration in the imaging lens according to the present embodiment is also described together.

It is to be noted that the effects described in the present specification are mere examples and are non-limiting. Further, any other effect may be provided.

According to the imaging lens of the present embodiment, with a seven-lens overall configuration, optimization of a configuration of each lens is attained. Hence, it is possible to correct various aberrations optimally, while allowing for size reduction and an increase in aperture size.

In the imaging lens of the present embodiment, as described below, it is desirable that optimization of refractive power arrangement, optimization of lens shape with the effective use of an aspheric surface, and optimization of a lens material, and the like be carried out.

In the imaging lens of the present embodiment, it is desirable that the seventh lens L7 have an aspheric shape having an inflection point in the lens surface on the image plane side. In other words, it is desirable that in the seventh lens L7, the lens surface on the image plane side has the aspheric shape having the inflection point, to allow a concavo-convex shape to change halfway as goes from a center portion to a peripheral portion. Allowing the lens surface on the image plane side of the seventh lens L7 to have a concave shape in the vicinity of the optical axis and to have a convex shape in the peripheral portion makes it possible to reduce an angle of entrance into the image plane IMG of light emitted from the seventh lens L7.

It is desirable that the imaging lens according to the present embodiment satisfy the following conditional expression (1).

1.1<TTL/f12<1.8 (1)

where “TTL” is a distance along the optical axis from a vertex of a surface on the object side of the first lens L1 to the image plane, and “f12” is a composite focal length of the first lens L1 and the second lens L2.

The conditional expression (1) defines a ratio between the distance along the optical axis from the vertex of the surface on the object side of the first lens L1 to the image plane and the composite focal length of the first lens L1 and the second lens L2. Satisfying the conditional expression (1) makes it possible to ensure small size and optimal performance. In a case where it is greater than an upper limit of the conditional expression (1), the composite focal length of the first lens L1 and the second lens L2 becomes short. This allows for the size reduction but strengthens refractive power with respect to an entering light beam, contributing to occurrence of a higher-order spherical aberration and a coma aberration, and causing difficulty in ensuring optical performance. In a case where it is smaller than a lower limit of the conditional expression (1), the composite focal length of the first lens L1 and the second lens L2 becomes long. This weakens the refractive power with respect to the entering light beam, contributing to an increase in a lens total length, and causing difficulty in achieving the size reduction.

Moreover, it is desirable that the imaging lens according to the present embodiment further satisfy the following conditional expression (2).

0.8<f1/f <273.0 (2)

where “f1” is a focal length of the first lens L1, and “f” is a focal length of a whole lens system.

The conditional expression (2) defines a ratio between the focal length of the first lens L1 and the focal length of the whole lens system. Satisfying the conditional expression (2) makes it possible to ensure the small size and the optimal performance. In a case where it is greater than an upper limit of the conditional expression (2), the focal length of the first lens L1 becomes long. This weakens the refractive power with respect to the entering light beam, contributing to the increase in the lens total length, and causing difficulty in achieving the size reduction. In a case where it is smaller than a lower limit of the conditional expression (2), the focal length of the first lens L1 becomes short. This strengthens the refractive power with respect to the entering light beam, leading to the size reduction, and easier correction on the coma aberration. However, sensitivity upon lens assembly becomes higher.

It is to be noted that it is more desirable that a numerical range of the conditional expression (2) be set as in the following conditional expression (2)′, to achieve the forgoing effects of the conditional expression (2) more optimally.

0.8 <f1/f <30.0 (2)′

Moreover, it is desirable that the imaging lens according to the present embodiment satisfy the following conditional expression (3).

0.6<f2/f <116.0 (3)

where “f2” is a focal length of the second lens L2, and “f” is the focal length of the whole lens system.

The conditional expression (3) defines a ratio between the focal length of the second lens L2 and the focal length of the whole lens system. Satisfying the conditional expression (3) makes it possible to ensure the small size and the optimal performance. In a case where it is greater than an upper limit of the conditional expression (3), the focal length of the second lens L2 becomes long. This weakens the refractive power with respect to the entering light beam, contributing to the increase in the lens total length, and causing difficulty in achieving the size reduction. In a case where it is smaller than a lower limit of the conditional expression (3), the focal length of the second lens L2 becomes short. This strengthens the refractive power with respect to the entering light beam, leading to the size reduction, and the easier correction on the coma aberration. However, the sensitivity upon the lens assembly becomes higher.

It is to be noted that it is more desirable that a numerical range of the conditional expression (3) be set as in the following conditional expression (3)′, to achieve the forgoing effects of the conditional expression (3) more optimally.

0.6<f2/f<1.4 (3)′

Moreover, it is desirable that the imaging lens according to the present embodiment satisfy the following conditional expression (4).

17.3<vd(L3)<28.5 (4)

where “vd(L3)” is an Abbe number of the third lens L3 with respect to a d-line.

The conditional expression (4) defines the Abbe number of the third lens L3. Satisfying the conditional expression (4) makes it possible to ensure optimal performance. In a case where it is greater than an upper limit of the conditional expression (4), sufficient refractive indexes with respect to an F-line and a g-line are not obtained. This causes an axial chromatic aberration to be controlled inadequately. In a case where it is smaller than a lower limit of the conditional expression (4), refractive indexes with respect to the F-line and the g-line becomes excessive. This causes the axial chromatic aberration to be controlled inadequately.

Moreover, it is desirable that the imaging lens according to the present embodiment satisfy the following conditional expression (5).

1.4<|f3/f12|<5.1 (5)

where “f3” is a focal length of the third lens L3, and “f12” is the composite focal length of the first lens L1 and the second lens L2.

The conditional expression (5) defines a ratio between the focal length of the third lens L3 and the composite focal length of the first lens L1 and the second lens L2. Satisfying the conditional expression (5) makes it possible to ensure the small size and the optimal performance. In a case where it is greater than an upper limit of the conditional expression (5), the composite focal length of the first lens L1 and the second lens L2 becomes short. This strengthens the refractive power with respect to the entering light beam, making it advantageous in the size reduction. However, it becomes difficult in making well-balanced correction on the aberrations. In a case where it is smaller than a lower limit of the conditional expression (5), the composite focal length of the first lens L1 and the second lens L2 becomes long. This weakens the refractive power with respect to the entering light beam, making it advantageous in suppression of occurrence of the aberrations. However, achieving the size reduction becomes difficult.

Moreover, it is desirable that the imaging lens according to the present embodiment further satisfy the following conditional expression (6).

4.2<f3/f<−1.3 (6)

where “f3” is the focal length of the third lens L3, and “f” is the focal length of the whole lens system.

The conditional expression (6) defines a ratio between the focal length of the third lens L3 and the focal length of the whole lens system. Satisfying the conditional expression (6) makes it possible to ensure the small size and the optimal performance. In a case where it is greater than an upper limit of the conditional expression (6), the focal length of the third lens L3 becomes short. This strengthens the refractive power with respect to the entering light beam, leading to the size reduction and the easier correction on the coma aberration. However, the sensitivity upon the lens assembly becomes higher. In a case where it is smaller than a lower limit of the conditional expression (6), the focal length of the third lens L3 becomes long. This weakens the refractive power with respect to the entering light beam, contributing to the increase in the lens total length, and causing difficulty in achieving size reduction.

Moreover, it is desirable that the imaging lens according to the present embodiment further satisfy the following conditional expression (7).

0.0<f3/f456<1.5 (7)

where “f3” is the focal length of the third lens L3, and “f456” is a composite focal length of the fourth lens L4, the fifth lens L5, and the sixth lens L6.

The conditional expression (7) defines a ratio between the focal length of the third lens L3 and the composite focal length of the fourth lens L4, the fifth lens L5, and the sixth lens L6. Satisfying the conditional expression (7) makes it possible to ensure the small size and the optimal performance. In a case where it is greater than an upper limit of the conditional expression (7), the composite focal length of the fourth lens L4, the fifth lens L5, and the sixth lens L6 becomes short. This strengthens the refractive power with respect to the entering light beam, contributing to excessive correction on an off-axis aberration. In particular, correction on the coma aberration and a field curvature becomes difficult. Moreover, there arises a disadvantage to reduction in the lens total length, causing difficulty in achieving the size reduction. In a case where it is smaller than a lower limit of the conditional expression (7), the composite focal length of the fourth lens L4, the fifth lens L5, and the sixth lens L6 becomes long. This weakens the refractive power with respect to the entering light beam, contributing to insufficient correction on the off-axis aberration. In particular, the correction on the coma aberration and the field curvature becomes difficult. Moreover, while being advantageous in the reduction in the lens total length, the sensitivity upon the lens assembly becomes higher.

Moreover, it is desirable that the imaging lens according to the present embodiment further satisfy the following conditional expression (8).

0.0<f3/f4567<2.2 (8)

where “f3” is the focal length of the third lens L3, and “f4567” is a composite focal length of the fourth lens L4, the fifth lens L5, the sixth lens L6, and the seventh lens L7.

The conditional expression (8) defines a ratio between the focal length of the third lens L3 and the composite focal length of the fourth lens L4, the fifth lens L5, the sixth lens L6, and the seventh lens L7. Satisfying the conditional expression (8) makes it possible to ensure the small size and the optimal performance. In a case where it is greater than an upper limit of the conditional expression (8), the composite focal length of the fourth lens L4, the fifth lens L5, the sixth lens L6, and the seventh lens L7 becomes short. This strengthens the refractive power with respect to the entering light beam, contributing to the excessive correction on the off-axis aberration. In particular, the correction on the coma aberration and the field curvature becomes difficult. Moreover, there arises the disadvantage to the reduction in the lens total length, causing difficulty in achieving the size reduction. In a case where it is smaller than a lower limit of the conditional expression (8), the composite focal length of the fourth lens L4, the fifth lens L5, the sixth lens L6, and the seventh lens L7 becomes long. This weakens the refractive power with respect to the entering light beam, contributing to the insufficient correction on the off-axis aberration. In particular, the correction on the coma aberration and the field curvature becomes difficult. Moreover, while being advantageous in the reduction in the lens total length, the sensitivity upon the lens assembly becomes higher.

Moreover, it is desirable that the imaging lens according to the present embodiment further satisfy the following conditional expression (9).

−470.0<f4/f<−2.3 (9)

where “f4” is a focal length of the fourth lens L4, and “f” is the focal length of the whole lens system.

The conditional expression (9) defines a ratio between the focal length of the fourth lens L4 and the focal length of the whole lens system. Satisfying the conditional expression (9) makes it possible to ensure the small size and the optimal performance. In a case where it is greater than an upper limit of the conditional expression (9), the focal length of the fourth lens L4 becomes short. This strengthens the refractive power with respect to the entering light beam, leading to the size reduction and the easier correction on the coma aberration. However, the sensitivity upon the lens assembly becomes higher. In a case where it is smaller than a lower limit of the conditional expression (9), the focal length of the fourth lens L4 becomes long. This weakens the refractive power with respect to the entering light beam, contributing to the increase in the lens total length, and causing difficulty in achieving size reduction.

It is to be noted that it is more desirable that a numerical range of the conditional expression (9) be set as in the following conditional expression (9)′, to achieve the forgoing effects of the conditional expression (9) more optimally.

−116.0<f4/f<−2.3 (9)′

It is to be noted that it is more desirable that the numerical range of the conditional expression (9) be set as in the following conditional expression (9)″, to achieve the forgoing effects of the conditional expression (9) even more optimally.

−85.0<f4/f<−2.3 (9)″

Moreover, it is desirable that the imaging lens according to the present embodiment further satisfy the following conditional expression (10).

1.7<|f7/f12|<274.0 (10)

where “f7” is a focal length of the seventh lens L7, and “f12” is the composite focal length of the first lens L1 and the second lens L2.

The conditional expression (10) defines a ratio between the focal length of the seventh lens L7 and the composite focal length of the first lens L1 and the second lens L2. Satisfying the conditional expression (10) makes it possible to ensure the small size and the optimal performance. In a case where it is greater than an upper limit of the conditional expression (10), the focal length of the seventh lens L7 becomes long. This weakens the refractive power with respect to the entering light beam, contributing to a gentle angle at which the light beam is bounced up. Thus, the lens total length becomes long, causing difficulty in achieving the size reduction. In a case where it is smaller than a lower limit of the conditional expression (10), the focal length of the seventh lens L7 becomes short. This strengthens the refractive power with respect to the entering light beam, contributing to a steep angle at which the light beam is bounced up. Thus, the correction on the off-axis aberration, in particular, the correction on a distortion becomes difficult.

It is to be noted that it is more desirable that a numerical range of the conditional expression (10) be set as in the following conditional expression (10)′, to achieve the forgoing effects of the conditional expression (10) more optimally.

1.7<|f7/f12|<28.0 (10)′

Moreover, it is desirable that the imaging lens according to the present embodiment further satisfy the following conditional expression (11).

0.5<|f1/f34567|<263.0 (11)

where “f1” is the focal length of the first lens L1, and “f34567” is a composite focal length of the third lens L3, the fourth lens L4, the fifth lens L5, the sixth lens L6, and the seventh lens L7.

The conditional expression (11) defines a ratio between the focal length of the first lens L1 and the composite focal length of the third lens L3, the fourth lens L4, the fifth lens L5, the sixth lens L6, and the seventh lens L7. Satisfying the conditional expression (11) makes it possible to ensure the small size and the optimal performance. In a case where it is greater than an upper limit of the conditional expression (11), the composite focal length of the third lens L3, the fourth lens L4, the fifth lens L5, the sixth lens L6, and the seventh lens L7 becomes short. This strengthens the refractive power with respect to the entering light beam, contributing to excessive correction on axial and off-axis aberrations. In particular, the correction on the spherical aberration and the coma aberration becomes difficult. Moreover, there arises the disadvantage to the reduction in the lens total length, causing difficulty in achieving the size reduction. In a case where it is smaller than a lower limit of the conditional expression (11), the composite focal length of the third lens L3, the fourth lens L4, the fifth lens L5, the sixth lens L6, and the seventh lens L7 becomes long. This weakens the refractive power with respect to the entering light beam, contributing to insufficient correction on the axial and off-axis aberrations. In particular, the correction on the spherical aberration and the coma aberration becomes difficult. Moreover, while being advantageous in the reduction in the lens total length, the sensitivity upon the lens assembly becomes higher.

It is to be noted that it is more desirable that a numerical range of the conditional expression (11) be set as in the following conditional expression (11)′, to achieve the forgoing effects of the conditional expression (11) more optimally.

0.5<|f1/f34567|<27.0 (11)′

Moreover, it is desirable that the imaging lens according to the present embodiment further satisfy the following conditional expression (12).

0.6<|f2/f34567|<79.8 (12)

where “f2” is the focal length of the second lens L2, and “f34567” is the composite focal length of the third lens L3, the fourth lens L4, the fifth lens L5, the sixth lens L6, and the seventh lens L7.

The conditional expression (12) defines a ratio between the focal length of the second lens L2 and the composite focal length of the third lens L3, the fourth lens L4, the fifth lens L5, the sixth lens L6, and the seventh lens L7. Satisfying the conditional expression (12) makes it possible to ensure the small size and the optimal performance. In a case where it is greater than an upper limit of the conditional expression (12), the composite focal length of the third lens L3, the fourth lens L4, the fifth lens L5, the sixth lens L6, and the seventh lens L7 becomes short. This strengthens the refractive power with respect to the entering light beam, contributing to the excessive correction on the axial and off-axis aberrations. In particular, the correction on the spherical aberration and the coma aberration becomes difficult. Moreover, there arises the disadvantage to the reduction in the lens total length, causing difficulty in achieving the size reduction. In a case where it is smaller than a lower limit of the conditional expression (12), the composite focal length of the third lens L3, the fourth lens L4, the fifth lens L5, the sixth lens L6, and the seventh lens L7 becomes long. This weakens the refractive power with respect to the entering light beam, contributing to the insufficient correction on the axial and off-axis aberrations. In particular, the correction on the spherical aberration and the coma aberration becomes difficult. Moreover, while being advantageous in the reduction in the lens total length, the sensitivity upon the lens assembly becomes higher.

Moreover, it is desirable that the imaging lens according to the present embodiment further satisfy the following conditional expression (13).

0.0<f3/f4<1.3 (13)

where “f3” is the focal length of the third lens L3, and “f4” is the focal length of the fourth lens L4.

The conditional expression (13) defines a ratio between the focal length of the third lens L3 and the focal length of the fourth lens L4. Satisfying the conditional expression (13) makes it possible to ensure the small size and the optimal performance. In a case where it is greater than an upper limit of the conditional expression (13), the focal length of the third lens L3 becomes long. This weakens the refractive power with respect to the entering light beam, contributing to the gentle angle at which the light beam is bounced up. This exerts great influences of distribution with respect to light beams at peripheral viewing angles, causing deterioration in a lateral chromatic aberration. Furthermore, the lens total length becomes long, causing difficulty in achieving the size reduction. In a case where it is smaller than a lower limit of the conditional expression (13), the focal length of the third lens L3 becomes short. This strengthens the refractive power with respect to the entering light beam, contributing to the steep angle at which the light beam is bounced up. Thus, the correction on the coma aberration and the field curvature becomes difficult.

Moreover, it is desirable that the imaging lens according to the present embodiment further satisfy the following conditional expression (14).

0.0<f3/f5<1.0 (14)

where “f3” is the focal length of the third lens L3, and “f5” is a focal length of the fifth lens L5.

The conditional expression (14) defines a ratio between the focal length of the third lens L3 and the focal length of the fifth lens L5. Satisfying the conditional expression (14) makes it possible to ensure the small size and the optimal performance. In a case where it is greater than an upper limit of the conditional expression (14), the focal length of the third lens L3 becomes long. This weakens the refractive power with respect to the entering light beam, contributing to the gentle angle at which the light beam is bounced up. This exerts great influences of distribution with respect to the light beams at the peripheral viewing angles, causing the deterioration in the lateral chromatic aberration. Furthermore, the lens total length becomes long, causing difficulty in achieving the size reduction. In a case where it is smaller than a lower limit of the conditional expression (14), the focal length of the third lens L3 becomes short. This strengthens the refractive power with respect to the entering light beam, contributing to the steep angle at which the light beam is bounced up. Thus, the correction on the coma aberration and the field curvature becomes difficult.

Moreover, it is desirable that the imaging lens according to the present embodiment further satisfy the following conditional expression (15).

−58.1<f45/f<−2.2 (15)

where “f45” is a composite focal length of the fourth lens L4 and the fifth lens L5, and “f” is the focal length of the whole lens system.

The conditional expression (15) defines a ratio between the composite focal length of the fourth lens L4 and the fifth lens L5, and the focal length of the whole lens system. Satisfying the conditional expression (15) makes it possible to ensure the small size and the optimal performance. In a case where it is greater than an upper limit of the conditional expression (15), the composite focal length of the fourth lens L4 and the fifth lens L5 becomes short. This strengthens the refractive power with respect to the entering light beam, leading to the size reduction and the easier correction on the coma aberration. However, the sensitivity upon the lens assembly becomes higher. In a case where it is smaller than a lower limit of the conditional expression (15), the composite focal length of the fourth lens L4 and the fifth lens L5 becomes long. This weakens the refractive power with respect to the entering light beam, contributing to the increase in the lens total length, and causing difficulty in achieving the size reduction.

Moreover, in the imaging lens according to the present embodiment, it is desirable that the aperture stop St be disposed between a lens surface on the object side of the first lens L1 and a lens surface on the image plane side of the first lens L1. In the case where the aperture stop St is disposed between the lens surface on the object side of the first lens L1 and the lens surface on the image plane side of the first lens L1, a spread of light beams entering the first lens L1 is suppressed. This allows for compatibility of both the aberration correction and improvement in flare caused by the first lens L1. However, the aperture stop St may be disposed at other positions. For example, the aperture stop St may be disposed between the lens surface of the image plane side of the first lens L1 and a lens surface on the image plane side of the second lens L2. In the case where the aperture stop St is disposed between the lens surface on the image plane side of the first lens L1 and the lens surface on the image plane side of the second lens L2, a spread of light beams entering the second lens L2 is suppressed. This allows for compatibility of both the aberration correction and improvement in flare caused by the second lens L2.

### 3. EXAMPLE OF APPLICATION TO IMAGING APPARATUS

Next, description is given of an example of application of the imaging lens according to the present embodiment to an imaging apparatus.

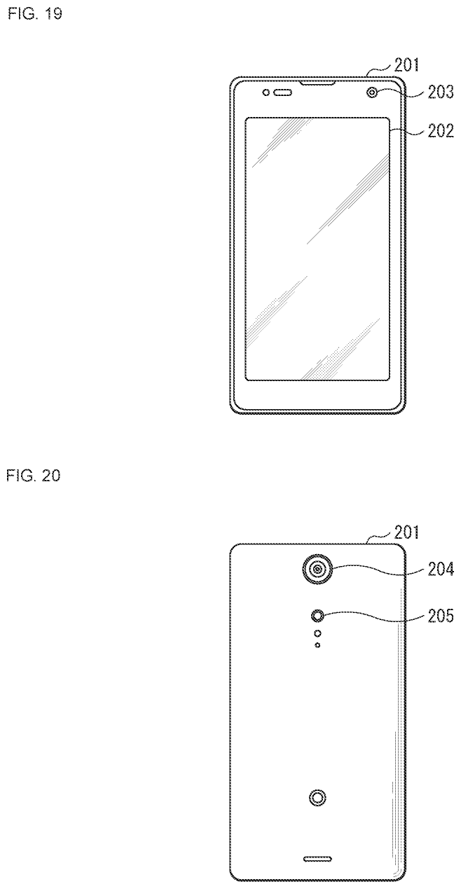

FIGS. 19 and 20 illustrate a configuration example of an imaging apparatus to which the imaging lens according to the present embodiment is applied. The configuration example is an example of a mobile terminal device including the imaging apparatus (e.g., a mobile information terminal and a mobile phone terminal). The mobile terminal device includes a casing 201 of a substantially rectangular shape. On front surface side of the casing 201 (FIG. 19), a display unit 202 and a front camera unit 203 are provided. On rear surface side of the casing 201 (FIG. 20), a main camera unit 204 and a camera flash 205 are provided.

The display unit 202 includes, for example, a touchscreen that detects a contact state with a front surface to allow for various operations. Thus, the display unit 202 has a display function of displaying various pieces of information and an input function of allowing a user to make various input operations. The display unit 202 displays an operation state and various kinds of data such as an image captured by the front camera unit 203 and the main camera unit 204.

The imaging lens according to the present embodiment is applicable as a lens for a camera module of the imaging apparatus (the front camera unit 203 and the main camera unit 204) in the mobile terminal device as illustrated in, for example, FIGS. 19 and 20. In a case with the use of the imaging lens as the lens for the camera module as mentioned above, as illustrated in FIG. 1, the imaging element 101 such as CCD and CMOS is disposed in the vicinity of the image plane IMG of the imaging lens. The imaging element 101 outputs an imaging signal (an image signal) corresponding to an optical image formed by the imaging lens. In this case, as illustrated in, for example, FIG. 1, between a last-stage lens and the image plane IMG, the optical member such as the sealing glass SG protecting the imaging element and the various optical filters may be disposed. Moreover, the optical members such as the sealing glass SG and the various optical filters may be disposed at any positions insofar as they are disposed between the last-stage lens and the image plane IMG.

It is to be noted that the imaging lens according to the present embodiment is applicable to not only the mobile terminal device as described above, but also an imaging lens for other electronic apparatuses such as digital still cameras and digital video cameras. Further, it is generally applicable to a small-sized imaging apparatus using a solid-state imaging element such as CCD and CMOS, e.g., an optical sensor, a mobile module camera, and a WEB camera, or the like. Moreover, it is also applicable to, for example, a monitoring camera.

### WORKING EXAMPLES

### 4. NUMERICAL EXAMPLES OF LENSES

Next, specific numerical examples of the imaging lens according to the present embodiment are described.

Here, numerical examples are described where specific numerical values are applied to the imaging lenses 1 to 9 of the respective configuration examples illustrated in FIGS. 1 to 9.

It is to be noted that meanings and the like of symbols described in the following tables and description are as follows. “Si” indicates the ordinal number of the i-th surface numbered in an increasing order from a closest one to the object side. “Ri” indicates a value of a paraxial radius of curvature of the i-th surface (unit: mm). “Di” indicates a value of an on-axial spacing between the i-th surface and the (i+1)-th surface (unit: mm). “Ndi” indicates a value of a refractive index with respect to the d-line (a wavelength of 587.6 nm) of a material of an optical element including the i-th surface. “vdi” indicates a value of an Abbe number with respect to the d-line of the material of the optical element including the i-th surface. A portion in which the value of “Ri” is “∞” indicates a planar surface or a virtual surface. “Li” indicates attributes of a surface. In “Li”, for example, “L1R1” indicates the lens surface on the object side of the first lens L1. “L1R2” indicates the lens surface on the image plane side of the first lens L1. Similarly, in “Li”, “L2R1” indicates a lens surface on the object side of the second lens L2. “L2R2” indicates a lens surface on the image plane side of the second lens L2. The same applies to other lens surfaces.

Moreover, some of the lenses to be used in each of the numerical examples have a lens surface that includes an aspheric surface. An aspheric shape is defined by the following expression. It is to be noted that in each of the tables describing aspheric surface coefficients described later, “E−i” represents exponential notation with a base 10, i.e., “10⁻ⁱ”. For example, “0.12345E−05” represents “0.12345×10⁻⁵”.

### (Expression of Aspheric Surface)

Z=C·h²/{1+(1−(1+K)·C·h²)^{1/2}}+Σan·hⁿ

(n is an integer greater than or equal to 3) where “Z” is a depth of the aspheric surface, “C” is a paraxial curvature and equals to 1/R, “h” is a distance from the optical axis to the lens surface, “K” is an eccentricity (2nd order aspheric coefficient), and “An” is an n-th order aspheric coefficient.

### (Configuration Common to Numerical Examples)

The imaging lenses 1 to 9 to which the following Numerical examples are respectively applied each have the configuration satisfying the basic lens configuration as described above. In other words, the imaging lenses 1 to 9 each have the substantially seven-lens configuration in which the first lens L1, the second lens L2, the third lens L3, the fourth lens L4, the fifth lens L5, the sixth lens L6, and the seventh lens L7 are disposed in order from the object side toward the image plane side.

The first lens L1 has the positive refractive power in the vicinity of the optical axis. The second lens L2 has the positive refractive power in the vicinity of the optical axis. The third lens L3 has the negative refractive power in the vicinity of the optical axis. The fourth lens L4 has the negative refractive power in the vicinity of the optical axis. The fifth lens L5 has the negative refractive power in the vicinity of the optical axis. The sixth lens L6 has the negative refractive power in the vicinity of the optical axis. In the seventh lens L7, the lens surface on the image plane side has the aspheric shape having the inflection point. The seventh lens L7 has the positive or negative refractive power in the vicinity of the optical axis.

The aperture stop St is disposed between the lens surface on the object side of the first lens L1 and the lens surface on the image plane side of the first lens L1.

The sealing glass SG is disposed between the seventh lens L7 and the image plane IMG.

### Numerical Example 1

Table 1 describes basic lens data of Numerical example 1 in which specific numerical values are applied to the imaging lens 1 illustrated in FIG. 1. In the imaging lens 1 according to Numerical example 1, the seventh lens L7 has the negative refractive power in the vicinity of the optical axis.

In the imaging lens 1 according to Numerical example 1, both surfaces of each lens of the first lens L1 to the seventh lens L7 have aspheric shapes. Tables 2 and 3 describe values of coefficients representing the aspheric shapes.

Moreover, Table 4 describes values of a focal length f, an F-number, a total length, and a half angle of view ω of a whole lens system in the imaging lens 1 according to Numerical example 1. Table 5 describes values of a focal length of each of the first lens L1, the second lens L2, the third lens L3, the fourth lens L4, the fifth lens L5, the sixth lens L6, and the seventh lens L7.

TABLE 1
Example 1

| Si | Li | Ri | Di | Ndi | νdi |
|---|---|---|---|---|---|
| 1 | | | 0.400 | | |
| 2 | St | ∞ | −0.400 | | |
| 3 | L1R1 | 2.108 | 0.629 | 1.544 | 56.1 |
| 4 | L1R2 | 3.633 | 0.072 | | |
| 5 | L2R1 | 3.310 | 0.543 | 1.544 | 56.1 |
| 6 | L2R2 | 83.221 | 0.029 | | |
| 7 | L3R1 | 27.700 | 0.250 | 1.671 | 19.2 |
| 8 | L3R2 | 5.466 | 0.351 | | |
| 9 | L4R1 | 13.990 | 0.330 | 1.544 | 56.1 |
| 10 | L4R2 | 13.698 | 0.169 | | |
| 11 | L5R1 | 662.055 | 0.447 | 1.644 | 22.5 |
| 12 | L5R2 | 22.158 | 0.162 | | |
| 13 | L6R1 | 4.619 | 0.777 | 1.616 | 25.8 |
| 14 | L6R2 | 4.305 | 0.266 | | |
| 15 | L7R1 | 2.270 | 0.846 | 1.535 | 55.7 |
| 16 | L7R2 | 1.778 | 0.640 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 2
Example 1

| | Si | | | |
|---|---|---|---|---|
| | 3 | 4 | 5 | 6 |
| R | 2.108 | 3.633 | 3.310 | 83.221 |
| K | −3.8053E−02 | −1.0000E+01 | −8.6464E+00 | −1.0000E+01 |
| A3 | 0.0000E+00 | 0.0000E+00 | 0.0000E+00 | 0.0000E+00 |
| A4 | −8.9000E−03 | −2.5596E−02 | −7.5714E−03 | −5.4707E−02 |
| A5 | 0.0000E+00 | 0.0000E+00 | 0.0000E+00 | 0.0000E+00 |
| A6 | −8.4236E−03 | 1.5396E−03 | −6.9069E−03 | 1.2705E−01 |
| A7 | 0.0000E+00 | 0.0000E+00 | 0.0000E+00 | 0.0000E+00 |
| A8 | 4.7831E−03 | −3.1878E−02 | −8.2106E−03 | −1.8482E−01 |
| A9 | 0.0000E+00 | 0.0000E+00 | 0.0000E+00 | 0.0000E+00 |
| A10 | −5.8996E−03 | 3.4677E−02 | 1.7474E−02 | 1.3163E−01 |
| A11 | 0.0000E+00 | 0.0000E+00 | 0.0000E+00 | 0.0000E+00 |
| A12 | 1.2222E−03 | −1.4145E−02 | −5.2799E−03 | −4.3676E−02 |
| A13 | 0.0000E+00 | 0.0000E+00 | 0.0000E+00 | 0.0000E+00 |
| A14 | 0.0000E+00 | 2.0406E−03 | 0.0000E+00 | 5.2103E−03 |
| A15 | 0.0000E+00 | 0.0000E+00 | 0.0000E+00 | 0.0000E+00 |
| A16 | 0.0000E+00 | 0.0000E+00 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |
| | 7 | 8 | 9 | 10 |
| R | 27.700 | 5.466 | 13.990 | 13.698 |
| K | 1.0000E+01 | −3.5803E+00 | 0.0000E+00 | 0.0000E+00 |
| A3 | 0.0000E+00 | 0.0000E+00 | 0.0000E+00 | 0.0000E+00 |
| A4 | −4.5707E−02 | −9.9386E−03 | −5.9876E−02 | −5.7621E−02 |
| A5 | 0.0000E+00 | 0.0000E+00 | 0.0000E+00 | 0.0000E+00 |
| A6 | 1.3949E−01 | 6.4043E−02 | 7.6111E−03 | 2.0232E−02 |
| A7 | 0.0000E+00 | 0.0000E+00 | 0.0000E+00 | 0.0000E+00 |
| A8 | −1.8391E−01 | −8.2579E−02 | −2.7636E−03 | −1.5685E−02 |
| A9 | 0.0000E+00 | 0.0000E+00 | 0.0000E+00 | 0.0000E+00 |
| A10 | 1.2110E−01 | 6.8200E−02 | −6.8883E−03 | −3.5074E−03 |
| A11 | 0.0000E+00 | 0.0000E+00 | 0.0000E+00 | 0.0000E+00 |
| A12 | −3.4983E−02 | −2.9895E−02 | 5.4907E−03 | 4.4296E−03 |
| A13 | 0.0000E+00 | 0.0000E+00 | 0.0000E+00 | 0.0000E+00 |
| A14 | 3.4053E−03 | 6.7358E−03 | 0.0000E+00 | 0.0000E+00 |
| A15 | 0.0000E+00 | 0.0000E+00 | 0.0000E+00 | 0.0000E+00 |
| A16 | 1.9930E−05 | 7.5117E−05 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

TABLE 3
Example 1

| | Si | | | |
|---|---|---|---|---|
| | 11 | 12 | 13 | 14 |
| R | 662.055 | 22.158 | 4.619 | 4.305 |
| K | −1.0000E+01 | −1.0000E+01 | 2.4516E+00 | 1.7684E+00 |
| A3 | −1.1253E−01 | −1.9307E−02 | 4.0392E−02 | −3.3500E−02 |
| A4 | 4.1214E−01 | 3.4038E−02 | −5.3667E−02 | −6.2484E−02 |
| A5 | −9.5058E−01 | 3.9442E−02 | 1.9150E−02 | 1.3600E−01 |
| A6 | 1.3940E+00 | −4.7633E−01 | −6.7076E−02 | −1.1170E−01 |
| A7 | −2.2940E+00 | 6.2083E−01 | 3.2922E−02 | 2.5712E−02 |
| A8 | 4.0667E+00 | −2.0276E−01 | −4.8053E−03 | 4.8875E−03 |
| A9 | −4.4393E+00 | −6.3639E−02 | 1.6542E−02 | −1.7676E−03 |
| A10 | 1.7058E+00 | −5.0744E−02 | −1.2440E−02 | 1.4927E−04 |
| A11 | 9.1638E−01 | 8.5993E−02 | −2.8514E−03 | −1.3535E−04 |
| A12 | −7.2280E−01 | 1.2794E−02 | 3.3651E−03 | −1.0633E−04 |
| A13 | −2.0869E−01 | −3.0108E−02 | −5.6424E−05 | 4.4468E−05 |
| A14 | 1.3037E−01 | −7.0986E−03 | −2.1126E−04 | 1.5763E−05 |
| A15 | 1.0817E−01 | 1.2849E−02 | 2.3779E−05 | −5.6792E−06 |
| A16 | −5.1629E−02 | −3.0976E−03 | −1.8936E−06 | 7.0280E−09 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

| | 15 | 16 |
|---|---|---|
| R | 2.270 | 1.778 |
| K | −6.3056E−01 | −1.2478E+00 |
| A3 | −3.0057E−02 | 2.6573E−02 |
| A4 | −2.1423E−01 | −2.3850E−01 |
| A5 | 8.5045E−02 | 1.2588E−01 |
| A6 | 9.7181E−03 | 9.8066E−05 |
| A7 | −4.7294E−03 | −2.0576E−02 |
| A8 | −2.8676E−03 | 4.7862E−03 |
| A9 | 7.7228E−04 | 3.7279E−04 |
| A10 | 2.8687E−04 | −2.6796E−06 |
| A11 | −4.1727E−05 | −3.7908E−05 |
| A12 | −3.3894E−05 | −2.1106E−05 |
| A13 | 5.1068E−08 | 4.8304E−06 |
| A14 | 1.8132E−06 | 1.0269E−06 |
| A15 | 6.2217E−07 | −1.2108E−07 |
| A16 | −1.7814E−07 | −2.6962E−08 |
| A17 | 0.0000E+00 | 0.0000E+00 |
| A18 | 0.0000E+00 | 0.0000E+00 |
| A19 | 0.0000E+00 | 0.0000E+00 |
| A20 | 0.0000E+00 | 0.0000E+00 |

TABLE 4
Example 1

| | |
|---|---|
| f | 4.69 |
| F-number | 1.6 |
| Total length | 5.821 |
| ω | 39.7 |

TABLE 5
Example 1

| | Focal length |
|---|---|
| L1 | 8.06 |
| L2 | 6.32 |
| L3 | −10.19 |
| L4 | −2004.60 |
| L5 | −35.61 |
| L6 | −1811.81 |
| L7 | −38.27 |

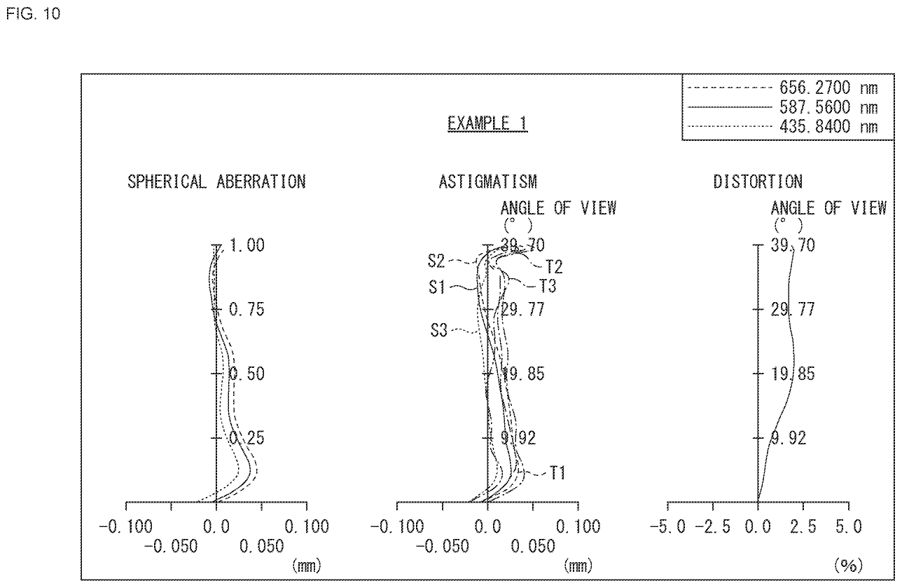

FIG. 10 illustrates various aberrations in Numerical example 1 described above. FIG. 10 illustrates a spherical aberration, an astigmatism (field curvature), and a distortion as the various aberrations. These aberration diagrams each illustrate the aberrations with the d-line (the wavelength of 587.6 nm) as a reference wavelength. The spherical aberration diagram and the astigmatism diagram also illustrate the aberrations with respect to a g-line (a wavelength of 435.84 nm) and a C-line (a wavelength of 656.27 nm). In the astigmatism diagram, “S” indicates a value on a sagittal image plane, and “T” indicates a value on a tangential image plane. The same applies to aberration diagrams in other numerical examples described below.

As can be seen from each of the aberration diagrams, the imaging lens 1 according to Numerical example 1 has the various aberrations corrected optimally while being small-sized with a great aperture. It is clear that the imaging lens 1 according to Numerical example 1 has superior image forming performance.

### Numerical Example 2

Table 6 describes basic lens data of Numerical example 2 in which specific numerical values are applied to the imaging lens 2 illustrated in FIG. 2. In the imaging lens 2 according to Numerical example 2, the seventh lens L7 has the negative refractive power in the vicinity of the optical axis.

In the imaging lens 2 according to Numerical example 2, both surfaces of each lens of the first lens L1 to the seventh lens L7 have aspheric shapes. Tables 7 and 8 describe values of coefficients representing the aspheric shapes.

Moreover, Table 9 describes values of a focal length f, an F-number, a total length, and a half angle of view ω of a whole lens system in the imaging lens 2 according to Numerical example 2. Table 10 describes values of a focal length of each of the first lens L1, the second lens L2, the third lens L3, the fourth lens L4, the fifth lens L5, the sixth lens L6, and the seventh lens L7.

TABLE 6
Example 2

| Si | Li | Ri | Di | Ndi | νdi |
|---|---|---|---|---|---|
| 1 | | | 0.370 | | |
| 2 | St | ∞ | −0.370 | | |
| 3 | L1R1 | 2.096 | 0.560 | 1.544 | 56.1 |
| 4 | L1R2 | 1.905 | 0.048 | | |
| 5 | L2R1 | 1.789 | 0.592 | 1.544 | 56.1 |
| 6 | L2R2 | 192.073 | 0.025 | | |
| 7 | L3R1 | 33.110 | 0.250 | 1.671 | 19.2 |
| 8 | L3R2 | 5.350 | 0.385 | | |
| 9 | L4R1 | −30.819 | 0.345 | 1.544 | 56.1 |
| 10 | L4R2 | −31.925 | 0.168 | | |
| 11 | L5R1 | 39.729 | 0.442 | 1.644 | 22.5 |
| 12 | L5R2 | 14.488 | 0.224 | | |
| 13 | L6R1 | 4.702 | 0.724 | 1.616 | 25.8 |
| 14 | L6R2 | 4.317 | 0.263 | | |
| 15 | L7R1 | 2.098 | 0.801 | 1.535 | 55.7 |
| 16 | L7R2 | 1.644 | 0.654 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 7
Example 2

| | Si | | | |
|---|---|---|---|---|
| | 3 | 4 | 5 | 6 |
| R | 2.096 | 1.905 | 1.789 | 192.073 |
| K | −1.0540E−01 | −6.2331E+00 | −5.4028E+00 | −9.8019E+00 |
| A3 | 0.0000E+00 | 0.0000E+00 | 0.0000E+00 | 0.0000E+00 |
| A4 | −1.3300E−02 | −5.8269E−02 | −3.4580E−02 | −5.5615E−02 |
| A5 | 0.0000E+00 | 0.0000E+00 | 0.0000E+00 | 0.0000E+00 |
| A6 | −3.5727E−03 | 4.8065E−03 | −2.4576E−02 | 1.2792E−01 |
| A7 | 0.0000E+00 | 0.0000E+00 | 0.0000E+00 | 0.0000E+00 |
| A8 | 5.7814E−03 | −3.2782E−02 | −1.0185E−02 | −2.0958E−01 |
| A9 | 0.0000E+00 | 0.0000E+00 | 0.0000E+00 | 0.0000E+00 |
| A10 | −6.6780E−03 | 4.1203E−02 | 2.2404E−02 | 1.5381E−01 |
| A11 | 0.0000E+00 | 0.0000E+00 | 0.0000E+00 | 0.0000E+00 |
| A12 | 1.6437E−03 | −1.7690E−02 | −5.7899E−03 | −5.2967E−02 |
| A13 | 0.0000E+00 | 0.0000E+00 | 0.0000E+00 | 0.0000E+00 |
| A14 | 0.0000E+00 | 3.0773E−03 | 0.0000E+00 | 6.8447E−03 |
| A15 | 0.0000E+00 | 0.0000E+00 | 0.0000E+00 | 0.0000E+00 |
| A16 | 0.0000E+00 | 0.0000E+00 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |
| | 7 | 8 | 9 | 10 |
| R | 33.110 | 5.350 | −30.819 | −31.925 |
| K | −9.9786E+00 | 5.7343E+00 | 0.0000E+00 | 0.0000E+00 |
| A3 | 0.0000E+00 | 0.0000E+00 | 0.0000E+00 | 0.0000E+00 |
| A4 | −3.0080E−02 | 2.6962E−03 | −3.7282E−02 | −5.0175E−02 |
| A5 | 0.0000E+00 | 0.0000E+00 | 0.0000E+00 | 0.0000E+00 |
| A6 | 1.5738E−01 | 6.8232E−02 | 6.8619E−03 | 1.7293E−02 |
| A7 | 0.0000E+00 | 0.0000E+00 | 0.0000E+00 | 0.0000E+00 |
| A8 | −2.1310E−01 | −9.9583E−02 | 5.2633E−04 | −1.2129E−02 |
| A9 | 0.0000E+00 | 0.0000E+00 | 0.0000E+00 | 0.0000E+00 |
| A10 | 1.3850E−01 | 7.9960E−02 | −8.3784E−03 | −1.8971E−03 |
| A11 | 0.0000E+00 | 0.0000E+00 | 0.0000E+00 | 0.0000E+00 |
| A12 | −4.0551E−02 | −3.6055E−02 | 6.6098E−03 | 4.3802E−03 |
| A13 | 0.0000E+00 | 0.0000E+00 | 0.0000E+00 | 0.0000E+00 |
| A14 | 4.2559E−03 | 8.4134E−03 | 0.0000E+00 | 0.0000E+00 |
| A15 | 0.0000E+00 | 0.0000E+00 | 0.0000E+00 | 0.0000E+00 |
| A16 | 2.5761E−05 | 9.7090E−05 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

TABLE 8
Example 2

| | Si | | | |
|---|---|---|---|---|
| | 11 | 12 | 13 | 14 |
| R | 39.729 | 14.488 | 4.702 | 4.317 |
| K | −1.0000E+01 | 3.8034E+00 | −1.4578E+00 | 1.8051E+00 |
| A3 | −1.1556E−01 | −3.0885E−02 | 3.6757E−02 | −3.7333E−02 |
| A4 | 4.1979E−01 | 3.8324E−02 | −5.0064E−02 | −6.5468E−02 |
| A5 | −1.0302E+00 | 3.6120E−02 | 2.8704E−02 | 1.4500E−01 |
| A6 | 1.5161E+00 | −5.1978E−01 | −7.4873E−02 | −1.2152E−01 |
| A7 | −2.5397E+00 | 6.8829E−01 | 3.4589E−02 | 2.8522E−02 |
| A8 | 4.5865E+00 | −2.2871E−01 | −5.8435E−03 | 5.5016E−03 |
| A9 | −5.0890E+00 | −7.3169E−02 | 1.9161E−02 | −2.0330E−03 |
| A10 | 1.9901E+00 | −5.9214E−02 | −1.4289E−02 | 1.7383E−04 |
| A11 | 1.0873E+00 | 1.0215E−01 | −3.2658E−03 | −1.5923E−04 |
| A12 | −8.7260E−01 | 1.5589E−02 | 4.0990E−03 | −1.2737E−04 |
| A13 | −2.5635E−01 | −3.6858E−02 | −6.7619E−05 | 5.5043E−05 |
| A14 | 1.6280E−01 | −8.8072E−03 | −2.7286E−04 | 1.9813E−05 |
| A15 | 1.3748E−01 | 1.6319E−02 | 2.3909E−05 | −7.2192E−06 |
| A16 | −6.6670E−02 | −4.0463E−03 | −5.5144E−06 | −2.2173E−08 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

| | 15 | 16 |
|---|---|---|
| R | 2.098 | 1.644 |
| K | −6.6356E−01 | −1.2274E+00 |
| A3 | −3.7907E−02 | 1.8376E−02 |
| A4 | −2.2582E−01 | −2.5090E−01 |
| A5 | 9.1639E−02 | 1.3596E−01 |
| A6 | 1.0707E−02 | 4.5554E−04 |
| A7 | −5.2380E−03 | −2.2763E−02 |
| A8 | −3.2397E−03 | 5.3861E−03 |
| A9 | 8.8239E−04 | 4.2085E−04 |
| A10 | 3.3380E−04 | −5.5516E−06 |
| A11 | −4.9616E−05 | −4.5646E−05 |
| A12 | −4.0879E−05 | −2.5618E−05 |
| A13 | 9.0783E−08 | 5.9136E−06 |
| A14 | 2.2743E−06 | 1.2863E−06 |
| A15 | 7.9079E−07 | −1.4962E−07 |
| A16 | −2.3253E−07 | −3.2569E−08 |
| A17 | 0.0000E+00 | 0.0000E+00 |
| A18 | 0.0000E+00 | 0.0000E+00 |
| A19 | 0.0000E+00 | 0.0000E+00 |
| A20 | 0.0000E+00 | 0.0000E+00 |

TABLE 9
Example 2

| | |
|---|---|
| f | 4.69 |
| F-number | 1.6 |
| Total length | 5.791 |
| ω | 39.8 |

TABLE 10
Example 2

| | Focal length |
|---|---|
| L1 | 1164.00 |
| L2 | 3.32 |
| L3 | −9.54 |
| L4 | −1837.21 |
| L5 | −35.65 |
| L6 | −302.26 |
| L7 | −36.88 |

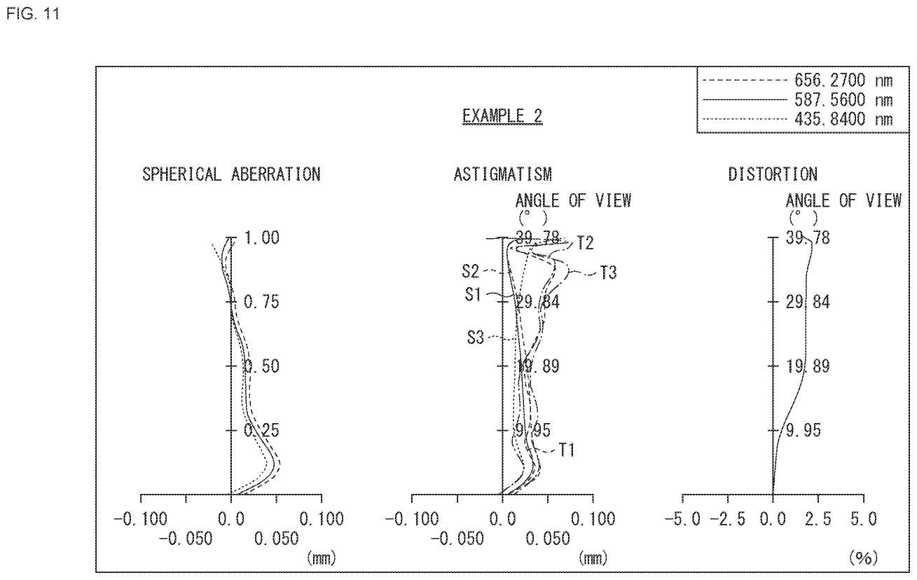

FIG. 11 illustrates various aberrations in Numerical example 2 described above.

As can be seen from each of the aberration diagrams, the imaging lens 2 according to Numerical example 2 has the various aberrations corrected optimally, while being small-sized with a great aperture. It is clear that the imaging lens 2 according to Numerical example 2 has superior image forming performance.

### Numerical Example 3

Table 11 describes basic lens data of Numerical example 3 in which specific numerical values are applied to the imaging lens 3 illustrated in FIG. 3. In the imaging lens 3 according to Numerical example 3, the seventh lens L7 has the negative refractive power in the vicinity of the optical axis.

In the imaging lens 3 according to Numerical example 3, both surfaces of each lens of the first lens L1 to the seventh lens L7 have aspheric shapes. Tables 12 and 13 describe values of coefficients representing the aspheric shapes.

Moreover, Table 14 describes values of a focal length f, an F-number, a total length, and a half angle of view ω of a whole lens system in the imaging lens 3 according to Numerical example 3. Table 15 describes values of a focal length of each of the first lens L1, the second lens L2, the third lens L3, the fourth lens L4, the fifth lens L5, the sixth lens L6, and the seventh lens L7.

TABLE 11
Example 3

| Si | Li | Ri | Di | Ndi | νdi |
|---|---|---|---|---|---|
| 1 | | | 0.340 | | |
| 2 | St | ∞ | −0.340 | | |
| 3 | L1R1 | 2.111 | 0.587 | 1.544 | 56.1 |
| 4 | L1R2 | 2.435 | 0.067 | | |
| 5 | L2R1 | 2.162 | 0.613 | 1.544 | 56.1 |
| 6 | L2R2 | 21.289 | 0.025 | | |
| 7 | L3R1 | 1000.000 | 0.250 | 1.671 | 19.2 |
| 8 | L3R2 | 11.637 | 0.432 | | |
| 9 | L4R1 | −8.183 | 0.384 | 1.544 | 56.1 |
| 10 | L4R2 | −9.786 | 0.097 | | |
| 11 | L5R1 | −17.015 | 0.397 | 1.644 | 22.5 |
| 12 | L5R2 | 46.212 | 0.174 | | |
| 13 | L6R1 | 5.079 | 0.812 | 1.616 | 25.8 |
| 14 | L6R2 | 4.408 | 0.221 | | |
| 15 | L7R1 | 2.160 | 0.830 | 1.535 | 55.7 |
| 16 | L7R2 | 1.798 | 0.620 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 12
Example 3

| | Si | | | |
|---|---|---|---|---|
| | 3 | 4 | 5 | 6 |
| R | 2.111 | 2.435 | 2.162 | 21.289 |
| K | −1.4547E−01 | −5.5061E+00 | −5.6444E+00 | 7.3415E+00 |
| A3 | 0.0000E+00 | 0.0000E+00 | 0.0000E+00 | 0.0000E+00 |
| A4 | −1.1500E−02 | −5.3073E−02 | −1.3993E−02 | −8.6363E−02 |
| A5 | 0.0000E+00 | 0.0000E+00 | 0.0000E+00 | 0.0000E+00 |
| A6 | −7.6258E−03 | 6.7811E−03 | −1.3986E−02 | 1.3723E−01 |
| A7 | 0.0000E+00 | 0.0000E+00 | 0.0000E+00 | 0.0000E+00 |
| A8 | 4.2284E−03 | −3.1643E−02 | −5.5837E−03 | −1.7997E−01 |
| A9 | 0.0000E+00 | 0.0000E+00 | 0.0000E+00 | 0.0000E+00 |
| A10 | −6.2536E−03 | 3.4280E−02 | 2.0056E−02 | 1.3141E−01 |
| A11 | 0.0000E+00 | 0.0000E+00 | 0.0000E+00 | 0.0000E+00 |
| A12 | 1.3696E−03 | −1.3982E−02 | −6.1352E−03 | −4.4517E−02 |
| A13 | 0.0000E+00 | 0.0000E+00 | 0.0000E+00 | 0.0000E+00 |
| A14 | 0.0000E+00 | 1.9839E−03 | 0.0000E+00 | 5.2669E−03 |
| A15 | 0.0000E+00 | 0.0000E+00 | 0.0000E+00 | 0.0000E+00 |
| A16 | 0.0000E+00 | 0.0000E+00 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |
| | 7 | 8 | 9 | 10 |
| R | 1000.000 | 11.637 | −8.183 | −9.786 |
| K | −1.0000E+01 | −7.7761E+00 | 0.0000E+00 | 0.0000E+00 |
| A3 | 0.0000E+00 | 0.0000E+00 | 0.0000E+00 | 0.0000E+00 |
| A4 | −3.3455E−02 | 3.4719E−02 | −4.6220E−02 | −9.3713E−02 |
| A5 | 0.0000E+00 | 0.0000E+00 | 0.0000E+00 | 0.0000E+00 |
| A6 | 1.3741E−01 | 5.3005E−02 | 1.1777E−02 | 4.7362E−02 |
| A7 | 0.0000E+00 | 0.0000E+00 | 0.0000E+00 | 0.0000E+00 |
| A8 | −1.8451E−01 | −9.1570E−02 | −1.8902E−03 | −1.3810E−02 |
| A9 | 0.0000E+00 | 0.0000E+00 | 0.0000E+00 | 0.0000E+00 |
| A10 | 1.2257E−01 | 7.2951E−02 | −7.9753E−03 | −5.3245E−03 |
| A11 | 0.0000E+00 | 0.0000E+00 | 0.0000E+00 | 0.0000E+00 |
| A12 | −3.5532E−02 | −2.9922E−02 | 5.5091E−03 | 3.5337E−03 |
| A13 | 0.0000E+00 | 0.0000E+00 | 0.0000E+00 | 0.0000E+00 |
| A14 | 3.4042E−03 | 6.7588E−03 | 0.0000E+00 | 0.0000E+00 |
| A15 | 0.0000E+00 | 0.0000E+00 | 0.0000E+00 | 0.0000E+00 |
| A16 | 1.9828E−05 | 7.5117E−05 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

TABLE 13
Example 3

| | Si | | | |
|---|---|---|---|---|
| | 11 | 12 | 13 | 14 |
| R | −17.015 | 46.212 | 5.079 | 4.408 |
| K | −1.0000E+01 | 1.0000E+01 | 3.3781E+00 | 1.8282E+00 |
| A3 | −1.0137E−01 | −2.8862E−02 | 2.1929E−02 | −4.4632E−02 |
| A4 | 3.5173E−01 | 3.9728E−02 | −4.0506E−02 | −7.2356E−02 |
| A5 | −9.3883E−01 | 3.3292E−02 | 1.5228E−02 | 1.4267E−01 |
| A6 | 1.4071E+00 | −4.7817E−01 | −6.8864E−02 | −1.1112E−01 |
| A7 | −2.2916E+00 | 6.2151E−01 | 3.3219E−02 | 2.5364E−02 |
| A8 | 4.0658E+00 | −2.0198E−01 | −4.4026E−03 | 4.7588E−03 |
| A9 | −4.4403E+00 | −6.3087E−02 | 1.6778E−02 | −1.7849E−03 |
| A10 | 1.7059E+00 | −5.0593E−02 | −1.2372E−02 | 1.5203E−04 |
| A11 | 9.1685E−01 | 8.5960E−02 | −2.8489E−03 | −1.3160E−04 |
| A12 | −7.2252E−01 | 1.2734E−02 | 3.3507E−03 | −1.0477E−04 |
| A13 | −2.0877E−01 | −3.0148E−02 | −6.7294E−05 | 4.4871E−05 |
| A14 | 1.3011E−01 | −7.1086E−03 | −2.1709E−04 | 1.5814E−05 |
| A15 | 1.0802E−01 | 1.2847E−02 | 2.3132E−05 | −5.7013E−06 |
| A16 | −5.1648E−02 | −3.0859E−03 | −5.0403E−07 | −1.0366E−08 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

| | 15 | 16 |
|---|---|---|
| R | 2.160 | 1.798 |
| K | −5.8900E−01 | −2.2233E+00 |
| A3 | −4.3681E−02 | 4.5696E−02 |
| A4 | −2.1250E−01 | −2.3391E−01 |
| A5 | 8.4646E−02 | 1.2733E−01 |
| A6 | 9.6375E−03 | 1.8763E−04 |
| A7 | −4.7131E−03 | −2.0644E−02 |
| A8 | −2.8548E−03 | 4.7524E−03 |
| A9 | 7.7626E−04 | 3.6298E−04 |
| A10 | 2.8745E−04 | −4.8096E−06 |
| A11 | −4.1912E−05 | −3.8225E−05 |
| A12 | −3.4073E−05 | −2.1091E−05 |
| A13 | −3.4038E−08 | 4.8557E−06 |
| A14 | 1.7848E−06 | 1.0375E−06 |
| A15 | 6.1754E−07 | −1.1817E−07 |
| A16 | −1.7551E−07 | −2.6539E−08 |
| A17 | 0.0000E+00 | 0.0000E+00 |
| A18 | 0.0000E+00 | 0.0000E+00 |
| A19 | 0.0000E+00 | 0.0000E+00 |
| A20 | 0.0000E+00 | 0.0000E+00 |

TABLE 14
Example 3

| | |
|---|---|
| f | 4.64 |
| F-number | 1.6 |
| Total length | 5.819 |
| ω | 39.6 |

TABLE 15
Example 3

| | Focal length |
|---|---|
| L1 | 17.80 |
| L2 | 4.37 |
| L3 | −17.55 |
| L4 | −100.30 |
| L5 | −19.26 |
| L6 | −100.54 |
| L7 | −99.83 |

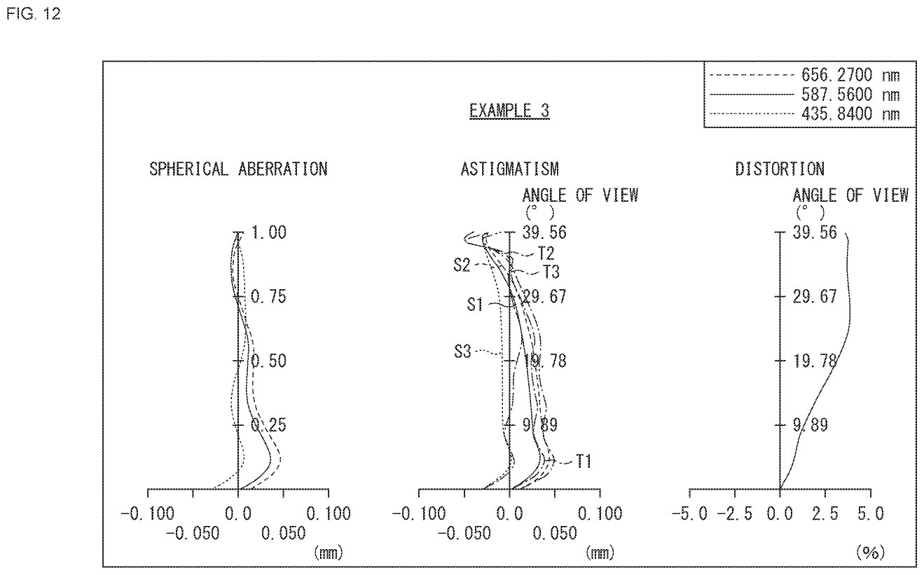

FIG. 12 describes various aberrations in Numerical example 3 described above.

As can be seen from each of the aberration diagrams, the imaging lens 3 according to Numerical example 3 has the various aberrations corrected optimally, while being small-sized with a great aperture. It is clear that the imaging lens 3 according to Numerical example 3 has superior image forming performance.

### Numerical Example 4

Table 16 describes basic lens data of Numerical example 4 in which specific numerical values are applied to the imaging lens 4 illustrated in FIG. 4. In the imaging lens 4 according to Numerical example 4, the seventh lens L7 has the positive refractive power in the vicinity of the optical axis.

In the imaging lens 4 according to Numerical example 4, both surfaces of each lens of the first lens L1 to the seventh lens L7 have aspheric shapes. Tables 17 and 18 describe values of coefficients representing the aspheric shapes.

Moreover, Table 19 describes values of a focal length f, an F-number, a total length, and a half angle of view ω of a whole lens system in the imaging lens 4 according to Numerical example 4. Table 20 describes values of a focal length of each of the first lens L1, the second lens L2, the third lens L3, the fourth lens L4, the fifth lens L5, the sixth lens L6, and the seventh lens L7.

TABLE 16
Example 4

| Si | Li | Ri | Di | Ndi | νdi |
|---|---|---|---|---|---|
| 1 | | | 0.180 | | |
| 2 | St | ∞ | −0.180 | | |
| 3 | L1R1 | 2.391 | 0.558 | 1.544 | 56.1 |
| 4 | L1R2 | 3.000 | 0.154 | | |
| 5 | L2R1 | 2.021 | 0.632 | 1.544 | 56.1 |
| 6 | L2R2 | −17.555 | 0.025 | | |
| 7 | L3R1 | −5.555 | 0.250 | 1.614 | 25.9 |
| 8 | L3R2 | 19.535 | 0.459 | | |
| 9 | L4R1 | −28.715 | 0.357 | 1.661 | 20.4 |
| 10 | L4R2 | 138.619 | 0.126 | | |
| 11 | L5R1 | −20.000 | 0.360 | 1.651 | 21.5 |
| 12 | L5R2 | −80.000 | 0.090 | | |
| 13 | L6R1 | 6.000 | 0.467 | 1.651 | 21.5 |
| 14 | L6R2 | 5.251 | 0.114 | | |
| 15 | L7R1 | 1.913 | 0.992 | 1.535 | 55.7 |
| 16 | L7R2 | 1.708 | 0.690 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 17
Example 4

| | Si | | | |
|---|---|---|---|---|
| | 3 | 4 | 5 | 6 |
| R | 2.391 | 3.000 | 2.021 | −17.555 |
| K | −2.2679E−01 | −6.3668E+00 | −5.4988E+00 | 5.0700E+00 |
| A3 | 0.0000E+00 | 0.0000E+00 | 0.0000E+00 | 0.0000E+00 |
| A4 | −3.3074E−02 | −8.7847E−02 | 5.4291E−04 | −1.5767E−02 |
| A5 | 0.0000E+00 | 0.0000E+00 | 0.0000E+00 | 0.0000E+00 |
| A6 | 1.9388E−02 | 5.9943E−02 | −2.3211E−02 | 6.2902E−02 |
| A7 | 0.0000E+00 | 0.0000E+00 | 0.0000E+00 | 0.0000E+00 |
| A8 | −2.3006E−02 | −1.5216E−01 | −1.9066E−02 | −1.5312E−01 |
| A9 | 0.0000E+00 | 0.0000E+00 | 0.0000E+00 | 0.0000E+00 |
| A10 | −3.3165E−02 | 2.7215E−01 | 1.1830E−01 | 1.2204E−02 |
| A11 | 0.0000E+00 | 0.0000E+00 | 0.0000E+00 | 0.0000E+00 |
| A12 | 9.3424E−02 | −3.0159E−01 | −2.0508E−01 | 3.2591E−01 |
| A13 | 0.0000E+00 | 0.0000E+00 | 0.0000E+00 | 0.0000E+00 |
| A14 | −9.4089E−02 | 2.1203E−01 | 2.1368E−01 | −4.8393E−01 |
| A15 | 0.0000E+00 | 0.0000E+00 | 0.0000E+00 | 0.0000E+00 |
| A16 | 4.8569E−02 | −9.2030E−02 | −1.3261E−01 | 3.2699E−01 |
| A17 | 0.0000E+00 | 0.0000E+00 | 0.0000E+00 | 0.0000E+00 |
| A18 | −1.2682E−02 | 2.2546E−02 | 4.4606E−02 | −1.1000E−01 |
| A19 | 0.0000E+00 | 0.0000E+00 | 0.0000E+00 | 0.0000E+00 |
| A20 | 1.3200E−03 | −2.3940E−03 | −6.2927E−03 | 1.4855E−02 |
| | 7 | 8 | 9 | 10 |
| R | −5.555 | 19.535 | −28.715 | 138.619 |
| K | 7.4201E+00 | −9.3224E+00 | 6.2324E+00 | 9.4626E+00 |
| A3 | 0.0000E+00 | 0.0000E+00 | −7.5901E−02 | −3.1276E−02 |
| A4 | 9.6844E−02 | 1.0499E−01 | 2.4228E−01 | 1.5750E−02 |
| A5 | 0.0000E+00 | 0.0000E+00 | −7.7317E−01 | 1.4870E−01 |
| A6 | 1.3673E−02 | −6.4034E−03 | 1.8369E+00 | −5.2326E−01 |
| A7 | 0.0000E+00 | 0.0000E+00 | −4.5144E+00 | 7.2038E−01 |
| A8 | −1.2777E−01 | −8.9728E−02 | 8.3460E+00 | −5.2535E−01 |
| A9 | 0.0000E+00 | 0.0000E+00 | −9.0186E+00 | −2.2839E−02 |
| A10 | 1.2972E−01 | 1.4314E−01 | 3.6786E+00 | 3.9436E−02 |
| A11 | 0.0000E+00 | 0.0000E+00 | 2.0339E+00 | 2.4847E−01 |
| A12 | −7.3335E−02 | −1.3921E−01 | −1.9191E+00 | 1.4748E−02 |
| A13 | 0.0000E+00 | 0.0000E+00 | −5.5977E−01 | −1.9944E−01 |
| A14 | 2.6027E−02 | 7.4454E−02 | 5.3617E−01 | −1.6517E−02 |
| A15 | 0.0000E+00 | 0.0000E+00 | 3.8673E−01 | 6.9585E−02 |
| A16 | −4.3175E−03 | −1.5790E−02 | −2.8119E−01 | 8.6765E−04 |
| A17 | 0.0000E+00 | 0.0000E+00 | −3.1777E−02 | 5.4529E−03 |
| A18 | 0.0000E+00 | 0.0000E+00 | 3.9860E−02 | −5.6638E−03 |
| A19 | 0.0000E+00 | 0.0000E+00 | 6.6046E−03 | −4.7469E−03 |
| A20 | 0.0000E+00 | 0.0000E+00 | −6.3053E−03 | 2.1484E−03 |

TABLE 18
Example 4

| | Si | | | |
|---|---|---|---|---|
| | 11 | 12 | 13 | 14 |
| R | −20.000 | −80.000 | 6.000 | 5.251 |
| K | 0.0000E+00 | 0.0000E+00 | 1.7568E+00 | 3.7949E+00 |
| A3 | 1.5377E−01 | 2.4609E−01 | 1.9584E−01 | −9.0217E−03 |
| A4 | −6.8077E−01 | −4.1574E−01 | −1.3229E−01 | −1.1602E−01 |
| A5 | 1.6057E+00 | 9.0216E−02 | −9.3083E−02 | 3.1162E−01 |
| A6 | −1.4619E+00 | 3.1501E−01 | 6.7036E−02 | −3.0965E−01 |
| A7 | 1.4804E−01 | −2.3162E−01 | 3.1225E−02 | 9.5364E−02 |
| A8 | 4.9877E−01 | 4.4122E−02 | −6.3793E−02 | 3.2253E−03 |
| A9 | −1.1904E−01 | −2.2128E−02 | 4.0285E−02 | −2.4989E−03 |
| A10 | −1.2182E−01 | 6.2967E−03 | −2.3907E−02 | −9.7294E−05 |
| A11 | −1.0796E−02 | 3.5504E−03 | 1.9695E−04 | −6.4788E−05 |
| A12 | 8.5181E−03 | 1.9498E−03 | 9.5313E−03 | −2.6329E−04 |
| A13 | 2.3868E−02 | −6.6767E−04 | −5.8118E−04 | 1.3707E−04 |
| A14 | 2.4392E−02 | 9.3040E−05 | −1.2203E−03 | 2.5547E−06 |
| A15 | −1.3192E−02 | −1.6939E−04 | −1.5225E−04 | −1.9882E−05 |
| A16 | −9.0548E−03 | −6.8195E−05 | −6.6339E−05 | −1.4639E−05 |
| A17 | −1.2625E−03 | −4.9619E−05 | −6.5630E−06 | 1.1123E−05 |
| A18 | 7.3188E−04 | −2.8168E−05 | 1.4032E−05 | −5.0665E−09 |
| A19 | 3.8688E−03 | 6.3691E−05 | 3.5973E−05 | −4.2543E−07 |
| A20 | −1.5316E−03 | −1.5411E−05 | −1.1594E−05 | −5.7765E−08 |

| | 15 | 16 |
|---|---|---|
| R | 1.913 | 1.708 |
| K | −6.1099E−01 | −1.0948E+00 |
| A3 | −6.7930E−02 | 4.5343E−02 |
| A4 | −2.8363E−01 | −3.2262E−01 |
| A5 | 1.0820E−01 | 1.8843E−01 |
| A6 | 4.1711E−02 | 5.4578E−03 |
| A7 | −3.0331E−03 | −4.4745E−02 |
| A8 | −8.9328E−03 | 1.1929E−02 |
| A9 | −1.1310E−03 | 2.1022E−03 |
| A10 | 8.3996E−04 | −8.1174E−04 |
| A11 | −2.0231E−04 | −3.7034E−05 |
| A12 | −8.9604E−05 | −1.7146E−05 |
| A13 | 7.0257E−05 | 2.2046E−06 |
| A14 | 4.0523E−05 | 2.5136E−06 |
| A15 | −5.3983E−06 | −L4954E−06 |
| A16 | −2.7787E−06 | 2.7692E−07 |
| A17 | −5.9667E−07 | 3.3798E−07 |
| A18 | −L7949E−07 | −4.7442E−08 |
| A19 | −8.1647E−08 | −3.2850E−08 |
| A20 | 6.5730E−08 | 5.8794E−09 |

TABLE 19
Example 4

| | |
|---|---|
| f | 4.21 |
| F-number | 1.6 |
| Total length | 5.585 |
| ω | 39.2 |

TABLE 20
Example 4

| | Focal length |
|---|---|
| L1 | 16.39 |
| L2 | 3.38 |
| L3 | −7.01 |
| L4 | −35.97 |
| L5 | −41.10 |
| L6 | −85.78 |
| L7 | 43.58 |

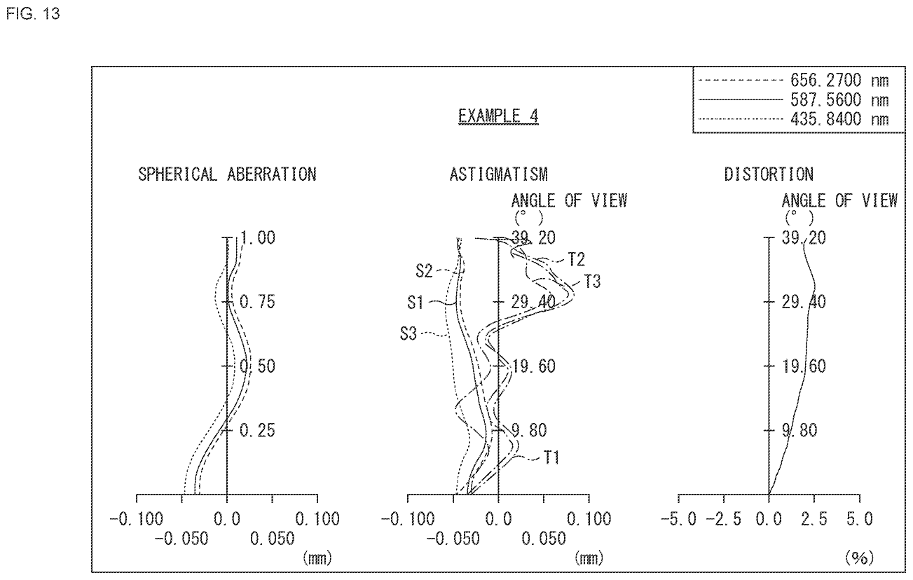

FIG. 13 describes various aberrations in Numerical example 4 described above.

As can be seen from each of the aberration diagrams, the imaging lens 4 according to Numerical example 4 has the various aberrations corrected optimally, while being small-sized with a great aperture. It is clear that the imaging lens 4 according to Numerical example 4 has superior image forming performance.

### Numerical Example 5

Table 21 describes basic lens data of Numerical example 5 in which specific numerical values are applied to the imaging lens 5 illustrated in FIG. 5. In the imaging lens 5 according to Numerical example 5, the seventh lens L7 has the negative refractive power in the vicinity of the optical axis.

In the imaging lens 5 according to Numerical example 5, both surfaces of each lens of the first lens L1 to the seventh lens L7 have aspheric shapes. Tables 22 and 23 describe values of coefficients representing the aspheric shapes.

Moreover, Table 24 describes values of a focal length f, an F-number, a total length, and a half angle of view ω of a whole lens system in the imaging lens 5 according to Numerical example 5. Table 25 describes values of a focal length of each of the first lens L1, the second lens L2, the third lens L3, the fourth lens L4, the fifth lens L5, the sixth lens L6, and the seventh lens L7.

TABLE 21
Example 5

| Si | Li | Ri | Di | Ndi | νdi |
|---|---|---|---|---|---|
| 1 | | | 0.400 | | |
| 2 | St | ∞ | −0.400 | | |
| 3 | L1R1 | 2.100 | 0.808 | 1.544 | 56.1 |
| 4 | L1R2 | 24.341 | 0.058 | | |
| 5 | L2R1 | 29.521 | 0.409 | 1.544 | 56.1 |
| 6 | L2R2 | 32.959 | 0.016 | | |
| 7 | L3R1 | 31.378 | 0.223 | 1.671 | 19.2 |
| 8 | L3R2 | 6.912 | 0.307 | | |
| 9 | L4R1 | 10.512 | 0.339 | 1.544 | 56.1 |
| 10 | L4R2 | 9.870 | 0.234 | | |
| 11 | L5R1 | 3473.402 | 0.430 | 1.644 | 22.5 |
| 12 | L5R2 | 49.838 | 0.224 | | |
| 13 | L6R1 | 4.644 | 0.686 | 1.616 | 25.8 |
| 14 | L6R2 | 4.317 | 0.300 | | |
| 15 | L7R1 | 2.246 | 0.832 | 1.535 | 55.7 |
| 16 | L7R2 | 1.766 | 0.674 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 22
Example 5

| | Si | | | |
|---|---|---|---|---|
| | 3 | 4 | 5 | 6 |
| R | 2.100 | 24.341 | 29.521 | 32.959 |
| K | 7.4881E−02 | −9.8701E+00 | 6.1672E+01 | −6.9241E+04 |
| A3 | 0.0000E+00 | 0.0000E+00 | 0.0000E+00 | 0.0000E+00 |
| A4 | −5.8203E−03 | −1.3373E−02 | 8.4560E−04 | −6.1377E−02 |
| A5 | 0.0000E+00 | 0.0000E+00 | 0.0000E+00 | 0.0000E+00 |
| A6 | −7.7716E−03 | 5.4386E−03 | −3.7691E−03 | 1.2880E−01 |
| A7 | 0.0000E+00 | 0.0000E+00 | 0.0000E+00 | 0.0000E+00 |
| A8 | 5.4860E−03 | −3.2101E−02 | −6.4380E−03 | −1.8311E−01 |
| A9 | 0.0000E+00 | 0.0000E+00 | 0.0000E+00 | 0.0000E+00 |
| A10 | −6.0733E−03 | 3.4641E−02 | 1.7419E−02 | 1.3194E−01 |
| A11 | 0.0000E+00 | 0.0000E+00 | 0.0000E+00 | 0.0000E+00 |
| A12 | 1.1302E−03 | −1.4187E−02 | −5.5379E−03 | −4.3916E−02 |
| A13 | 0.0000E+00 | 0.0000E+00 | 0.0000E+00 | 0.0000E+00 |
| A14 | 0.0000E+00 | 2.0053E−03 | 0.0000E+00 | 5.1829E−03 |
| A15 | 0.0000E+00 | 0.0000E+00 | 0.0000E+00 | 0.0000E+00 |
| A16 | 0.0000E+00 | 0.0000E+00 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |
| | 7 | 8 | 9 | 10 |
| R | 31.378 | 6.912 | 10.512 | 9.870 |
| K | −3.5619E+04 | −1.1809E+01 | 0.0000E+00 | 0.0000E+00 |
| A3 | 0.0000E+00 | 0.0000E+00 | 0.0000E+00 | 0.0000E+00 |
| A4 | −4.5652E−02 | −4.3791E−03 | −6.2097E−02 | −5.9697E−02 |
| A5 | 0.0000E+00 | 0.0000E+00 | 0.0000E+00 | 0.0000E+00 |
| A6 | 1.3728E−01 | 6.6655E−02 | 8.4281E−03 | 2.0402E−02 |
| A7 | 0.0000E+00 | 0.0000E+00 | 0.0000E+00 | 0.0000E+00 |
| A8 | −1.8459E−01 | −8.7541E−02 | −4.4945E−03 | −1.5375E−02 |
| A9 | 0.0000E+00 | 0.0000E+00 | 0.0000E+00 | 0.0000E+00 |
| A10 | 1.2129E−01 | 6.8421E−02 | −9.5741E−03 | −3.1599E−03 |
| A11 | 0.0000E+00 | 0.0000E+00 | 0.0000E+00 | 0.0000E+00 |
| A12 | −3.4613E−02 | −2.9895E−02 | 5.4907E−03 | 3.5111E−03 |
| A13 | 0.0000E+00 | 0.0000E+00 | 0.0000E+00 | 0.0000E+00 |
| A14 | 3.4053E−03 | 6.7358E−03 | 0.0000E+00 | 0.0000E+00 |
| A15 | 0.0000E+00 | 0.0000E+00 | 0.0000E+00 | 0.0000E+00 |
| A16 | 1.9930E−05 | 7.5117E−05 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

TABLE 23
Example 5

| | Si | | | |
|---|---|---|---|---|
| | 11 | 12 | 13 | 14 |
| R | 3473.402 | 49.838 | 4.644 | 4.317 |
| K | 4.4959E+06 | −7.5652E+03 | 3.5552E+00 | 1.7630E+00 |
| A3 | −1.0376E−01 | −1.6595E−02 | 2.6544E−02 | −2.4663E−02 |
| A4 | 3.9230E−01 | 2.9324E−02 | −3.6573E−02 | −7.1039E−02 |
| A5 | −9.5454E−01 | 3.7542E−02 | 1.5744E−02 | 1.3810E−01 |
| A6 | 1.3975E+00 | −4.7631E−01 | −6.9995E−02 | −1.1154E−01 |
| A7 | −2.2908E+00 | 6.2126E−01 | 3.2582E−02 | 2.5653E−02 |
| A8 | 4.0680E+00 | −2.0248E−01 | −4.5380E−03 | 4.8869E−03 |
| A9 | −4.4393E+00 | −6.3516E−02 | 1.6790E−02 | −1.7584E−03 |
| A10 | 1.7055E+00 | −5.0705E−02 | −1.2331E−02 | 1.5244E−04 |
| A11 | 9.1609E−01 | 8.6003E−02 | −2.8226E−03 | −1.3480E−04 |
| A12 | −7.2296E−01 | 1.2795E−02 | 3.3632E−03 | −1.0655E−04 |
| A13 | −2.0874E−01 | −3.0108E−02 | −6.2719E−05 | 4.4312E−05 |
| A14 | 1.3038E−01 | −7.0973E−03 | −2.1783E−04 | 1.5698E−05 |
| A15 | 1.0818E−01 | 1.2850E−02 | 2.0770E−05 | −5.6972E−06 |
| A16 | −5.1647E−02 | −3.0971E−03 | −3.0896E−06 | 8.4234E−09 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

| | 15 | 16 |
|---|---|---|
| R | 2.246 | 1.766 |
| K | −6.5502E−01 | −1.5534E+00 |
| A3 | −2.4503E−02 | 3.1030E−02 |
| A4 | −2.1429E−01 | −2.3495E−01 |
| A5 | 8.4468E−02 | 1.2691E−01 |
| A6 | 9.5529E−03 | 5.4227E−05 |
| A7 | −4.7671E−03 | −2.0661E−02 |
| A8 | −2.8763E−03 | 4.7548E−03 |
| A9 | 7.7023E−04 | 3.6460E−04 |
| A10 | 2.8641E−04 | −4.3460E−06 |
| A11 | −4.1787E−05 | −3.8145E−05 |
| A12 | −3.3876E−05 | −2.1105E−05 |
| A13 | 7.2895E−08 | 4.8493E−06 |
| A14 | 1.8265E−06 | 1.0368E−06 |
| A15 | 6.2877E−07 | −1.1710E−07 |
| A16 | −1.7521E−07 | −2.5535E−08 |
| A17 | 0.0000E+00 | 0.0000E+00 |
| A18 | 0.0000E+00 | 0.0000E+00 |
| A19 | 0.0000E+00 | 0.0000E+00 |
| A20 | 0.0000E+00 | 0.0000E+00 |

TABLE 24
Example 5

| | |
|---|---|
| f | 4.71 |
| F-number | 1.7 |
| Total length | 5.850 |
| ω | 39.0 |

TABLE 25
Example 5

| | |
|---|---|
| L1 | 4.18 |
| L2 | 500.00 |
| L3 | −13.27 |
| L4 | −364.84 |
| L5 | −78.49 |
| L6 | −500.00 |
| L7 | −38.88 |

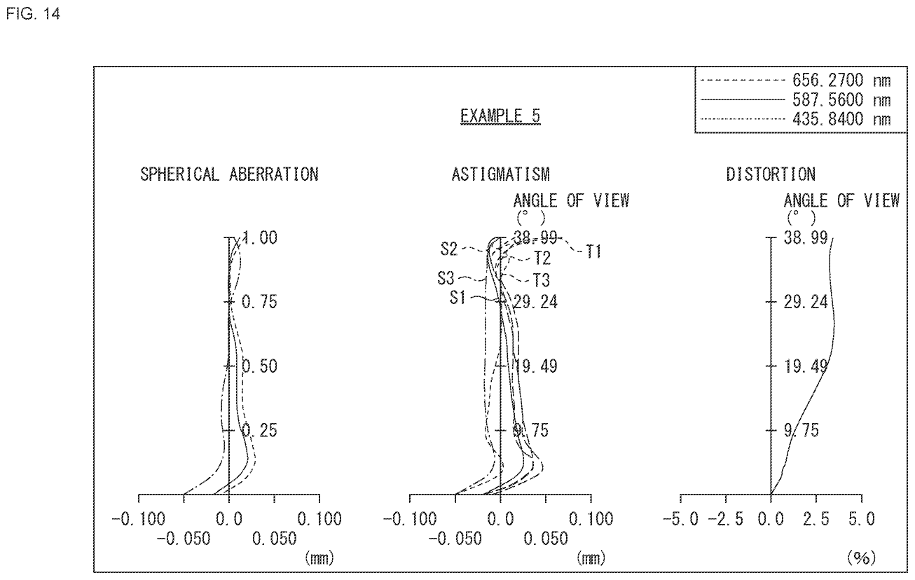

FIG. 14 describes various aberrations in Numerical example 5 described above.

As can be seen from each of the aberration diagrams, the imaging lens 5 according to Numerical example 5 has the various aberrations corrected optimally, while being small-sized with a great aperture. It is clear that the imaging lens 5 according to Numerical example 5 has superior image forming performance.

### Numerical Example 6

Table 26 describes basic lens data of Numerical example 6 in which specific numerical values are applied to the imaging lens 6 illustrated in FIG. 6. In the imaging lens 6 according to Numerical example 6, the seventh lens L7 has the negative refractive power in the vicinity of the optical axis.

In the imaging lens 6 according to Numerical example 6, both surfaces of each lens of the first lens L1 to the seventh lens L7 have aspheric shapes. Tables 27 and 28 describe values of coefficients representing the aspheric shapes.

Moreover, Table 29 describes values of a focal length f, an F-number, a total length, and a half angle of view ω of a whole lens system in the imaging lens 6 according to Numerical example 6. Table 30 describes values of a focal length of each of the first lens L1, the second lens L2, the third lens L3, the fourth lens L4, the fifth lens L5, the sixth lens L6, and the seventh lens L7.

TABLE 26
Example 6

| Si | Li | Ri | Di | Ndi | νdi |
|---|---|---|---|---|---|
| 1 | | | 0.370 | | |
| 2 | St | ∞ | −0.370 | | |
| 3 | L1R1 | 2.091 | 0.573 | 1.544 | 56.1 |
| 4 | L1R2 | 1.947 | 0.050 | | |
| 5 | L2R1 | 1.833 | 0.616 | 1.544 | 56.1 |
| 6 | L2R2 | −172.529 | 0.027 | | |
| 7 | L3R1 | 24.928 | 0.250 | 1.671 | 19.2 |
| 8 | L3R2 | 3.935 | 0.324 | | |
| 9 | L4R1 | 16.012 | 0.284 | 1.544 | 56.1 |
| 10 | L4R2 | 15.026 | 0.204 | | |
| 11 | L5R1 | 12.228 | 0.444 | 1.644 | 22.5 |
| 12 | L5R2 | 11.613 | 0.247 | | |
| 13 | L6R1 | 4.606 | 0.641 | 1.616 | 25.8 |
| 14 | L6R2 | 4.298 | 0.257 | | |
| 15 | L7R1 | 2.041 | 0.829 | 1.535 | 55.7 |
| 16 | L7R2 | 1.745 | 0.795 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 27
Example 6

| | Si | | | |
|---|---|---|---|---|
| | 3 | 4 | 5 | 6 |
| R | 2.091 | 1.947 | 1.833 | −172.529 |
| K | −1.1454E−01 | −5.9702E+00 | −5.3244E+00 | 1.4379E+04 |
| A3 | 0.0000E+00 | 0.0000E+00 | 0.0000E+00 | 0.0000E+00 |
| A4 | −1.3139E−02 | −5.4959E−02 | −3.1881E−02 | −5.2076E−02 |
| A5 | 0.0000E+00 | 0.0000E+00 | 0.0000E+00 | 0.0000E+00 |
| A6 | −3.0965E−03 | 4.7297E−03 | −2.3104E−02 | 1.1792E−01 |
| A7 | 0.0000E+00 | 0.0000E+00 | 0.0000E+00 | 0.0000E+00 |
| A8 | 4.8567E−03 | −2.9309E−02 | −9.2316E−03 | −1.8605E−01 |
| A9 | 0.0000E+00 | 0.0000E+00 | 0.0000E+00 | 0.0000E+00 |
| A10 | −5.8162E−03 | 3.5257E−02 | 1.9155E−02 | 1.3167E−01 |
| A11 | 0.0000E+00 | 0.0000E+00 | 0.0000E+00 | 0.0000E+00 |
| A12 | 1.4312E−03 | −1.4573E−02 | −4.8441E−03 | −4.3930E−02 |
| A13 | 0.0000E+00 | 0.0000E+00 | 0.0000E+00 | 0.0000E+00 |
| A14 | 0.0000E+00 | 2.4627E−03 | 0.0000E+00 | 5.5367E−03 |
| A15 | 0.0000E+00 | 0.0000E+00 | 0.0000E+00 | 0.0000E+00 |
| A16 | 0.0000E+00 | 0.0000E+00 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |
| | 7 | 8 | 9 | 10 |
| R | 24.928 | 3.935 | 16.012 | 15.026 |
| K | 1.0432E+02 | 6.3143E+00 | 0.0000E+00 | 0.0000E+00 |
| A3 | 0.0000E+00 | 0.0000E+00 | 0.0000E+00 | 0.0000E+00 |
| A4 | −2.6067E−02 | −1.4847E−03 | −3.6033E−02 | −4.8252E−02 |
| A5 | 0.0000E+00 | 0.0000E+00 | 0.0000E+00 | 0.0000E+00 |
| A6 | 1.4448E−01 | 6.2296E−02 | 3.9351E−03 | 1.6516E−02 |
| A7 | 0.0000E+00 | 0.0000E+00 | 0.0000E+00 | 0.0000E+00 |
| A8 | −1.8889E−01 | −8.9070E−02 | 1.6512E−03 | −1.2224E−02 |
| A9 | 0.0000E+00 | 0.0000E+00 | 0.0000E+00 | 0.0000E+00 |
| A10 | 1.1900E−01 | 6.9261E−02 | −6.7328E−03 | −2.1977E−03 |
| A11 | 0.0000E+00 | 0.0000E+00 | 0.0000E+00 | 0.0000E+00 |
| A12 | −3.3601E−02 | −2.9897E−02 | 5.4919E−03 | 3.9350E−03 |
| A13 | 0.0000E+00 | 0.0000E+00 | 0.0000E+00 | 0.0000E+00 |
| A14 | 3.4056E−03 | 6.7358E−03 | 0.0000E+00 | 0.0000E+00 |
| A15 | 0.0000E+00 | 0.0000E+00 | 0.0000E+00 | 0.0000E+00 |
| A16 | 1.9930E−05 | 7.5117E−05 | 0.0000E+00 | 0.0000E+00 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

TABLE 28
Example 6

| | Si | | | |
|---|---|---|---|---|
| | 11 | 12 | 13 | 14 |
| R | 12.228 | 11.613 | 4.606 | 4.298 |
| K | −4.5637E+02 | −3.9580E+02 | 1.7359E−01 | 1.7455E+00 |
| A3 | −1.0427E−01 | −2.0827E−02 | 3.7906E−02 | −2.9129E−02 |
| A4 | 4.0425E−01 | 3.4804E−02 | −4.5953E−02 | −6.6948E−02 |
| A5 | −9.5974E−01 | 3.4064E−02 | 2.3881E−02 | 1.3518E−01 |
| A6 | 1.3919E+00 | −4.7677E−01 | −6.9370E−02 | −1.1151E−01 |
| A7 | −2.2929E+00 | 6.2101E−01 | 3.1512E−02 | 2.5737E−02 |
| A8 | 4.0679E+00 | −2.0324E−01 | −4.9703E−03 | 4.8777E−03 |
| A9 | −4.4387E+00 | −6.4104E−02 | 1.6764E−02 | −1.7711E−03 |
| A10 | 1.7059E+00 | −5.0958E−02 | −1.2257E−02 | 1.5095E−04 |
| A11 | 9.1635E−01 | 8.5973E−02 | −2.7664E−03 | −1.3322E−04 |
| A12 | −7.2291E−01 | 1.2853E−02 | 3.3874E−03 | −1.0517E−04 |
| A13 | −2.0879E−01 | −3.0048E−02 | −5.8558E−05 | 4.4926E−05 |
| A14 | 1.3027E−01 | −7.0639E−03 | −2.1910E−04 | 1.5879E−05 |
| A15 | 1.0809E−01 | 1.2840E−02 | 1.9354E−05 | −5.6853E−06 |
| A16 | −5.1730E−02 | −3.1297E−03 | −3.5981E−06 | −2.2577E−08 |
| A17 | 0.0000E+00 | 0.0000E+00 | 0.0000E+00 | 0.0000E+00 |
| A18 | 0.0000E+00 | 0.0000E+00 | 0.0000E+00 | 0.0000E+00 |
| A19 | 0.0000E+00 | 0.0000E+00 | 0.0000E+00 | 0.0000E+00 |
| A20 | 0.0000E+00 | 0.0000E+00 | 0.0000E+00 | 0.0000E+00 |

| | 15 | 16 |
|---|---|---|
| R | 2.041 | 1.745 |
| K | −6.6609E−01 | −1.3468E+00 |
| A3 | −3.6216E−02 | 2.8280E−02 |
| A4 | −2.1413E−01 | −2.3880E−01 |
| A5 | 8.5431E−02 | 1.2682E−01 |
| A6 | 9.7808E−03 | 3.8885E−04 |
| A7 | −4.7353E−03 | −2.0534E−02 |
| A8 | −2.8743E−03 | 4.7846E−03 |
| A9 | 7.6999E−04 | 3.6920E−04 |
| A10 | 2.8642E−04 | −4.1795E−06 |
| A11 | −4.1729E−05 | −3.8344E−05 |
| A12 | −3.3845E−05 | −2.1203E−05 |
| A13 | 7.7439E−08 | 4.8175E−06 |
| A14 | 1.8205E−06 | 1.0288E−06 |
| A15 | 6.2161E−07 | −1.1845E−07 |
| A16 | −1.8038E−07 | −2.5528E−08 |
| A17 | 0.0000E+00 | 0.0000E+00 |
| A18 | 0.0000E+00 | 0.0000E+00 |
| A19 | 0.0000E+00 | 0.0000E+00 |
| A20 | 0.0000E+00 | 0.0000E+00 |

TABLE 29
Example 6

| | |
|---|---|
| f | 4.71 |
| F-number | 1.7 |
| Total length | 5.850 |
| ω | 39.4 |

TABLE 30
Example 6

| | Focal length |
|---|---|
| L1 | 130.82 |
| L2 | 3.34 |
| L3 | −7.00 |
| L4 | −500.00 |
| L5 | −500.00 |
| L6 | −500.00 |
| L7 | −897.59 |

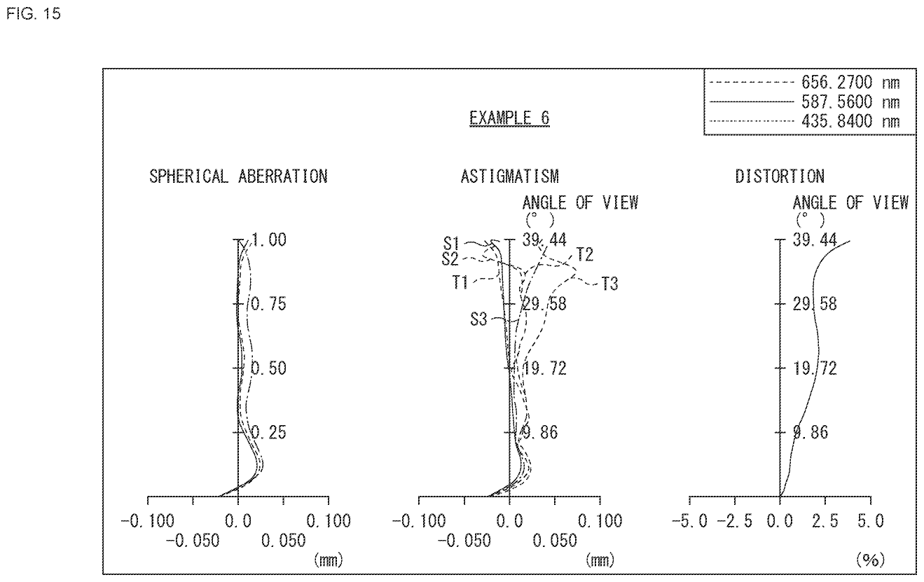

FIG. 15 describes various aberrations in Numerical example 6 described above.

As can be seen from each of the aberration diagrams, the imaging lens 6 according to Numerical example 6 has the various aberrations corrected optimally, while being small-sized with a great aperture. It is clear that the imaging lens 6 according to Numerical example 6 has superior image forming performance.

### Numerical Example 7

Table 31 describes basic lens data of Numerical example 7 in which specific numerical values are applied to the imaging lens 7 illustrated in FIG. 7. In the imaging lens 7 according to Numerical example 7, the seventh lens L7 has the positive refractive power in the vicinity of the optical axis.

In the imaging lens 7 according to Numerical example 7, both surfaces of each lens of the first lens L1 to the seventh lens L7 have aspheric shapes. Tables 32 and 33 describe values of coefficients representing the aspheric shapes.

Moreover, Table 34 describes values of a focal length f, an F-number, a total length, and a half angle of view ω of a whole lens system in the imaging lens 7 according to Numerical example 7. Table 35 describes values of a focal length of each of the first lens L1, the second lens L2, the third lens L3, the fourth lens L4, the fifth lens L5, the sixth lens L6, and the seventh lens L7.

TABLE 31
Example 7

| Si | Li | Ri | Di | Ndi | νdi |
|---|---|---|---|---|---|
| 1 | | | 0.180 | | |
| 2 | St | ∞ | −0.180 | | |
| 3 | L1R1 | 2.459 | 0.518 | 1.544 | 56.1 |
| 4 | L1R2 | 3.000 | 0.133 | | |
| 5 | L2R1 | 1.975 | 0.680 | 1.544 | 56.1 |
| 6 | L2R2 | 97.694 | 0.070 | | |
| 7 | L3R1 | −3.923 | 0.250 | 1.671 | 19.2 |
| 8 | L3R2 | −7.506 | 0.459 | | |
| 9 | L4R1 | −17.122 | 0.365 | 1.651 | 21.5 |
| 10 | L4R2 | 11.968 | 0.085 | | |
| 11 | L5R1 | −100.000 | 0.360 | 1.544 | 56.1 |
| 12 | L5R2 | −320.023 | 0.064 | | |
| 13 | L6R1 | 6.000 | 0.507 | 1.614 | 25.9 |
| 14 | L6R2 | 5.251 | 0.109 | | |
| 15 | L7R1 | 1.854 | 1.047 | 1.535 | 55.7 |
| 16 | L7R2 | 1.708 | 0.640 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 32
Example 7

| | Si | | | |
|---|---|---|---|---|
| | 3 | 4 | 5 | 6 |
| R | 2.459 | 3.000 | 1.975 | 97.694 |
| K | −2.2679E−01 | −6.3668E+00 | −5.4988E+00 | 5.0700E+00 |
| A3 | 0.0000E+00 | 0.0000E+00 | 0.0000E+00 | 0.0000E+00 |
| A4 | −2.7192E−02 | −9.6833E−02 | −3.0698E−03 | −3.4993E−02 |
| A5 | 0.0000E+00 | 0.0000E+00 | 0.0000E+00 | 0.0000E+00 |
| A6 | 2.8874E−03 | 6.3967E−02 | −2.1331E−02 | 4.2633E−02 |
| A7 | 0.0000E+00 | 0.0000E+00 | 0.0000E+00 | 0.0000E+00 |
| A8 | −1.6428E−03 | −1.5961E−01 | −4.0746E−02 | −1.3631E−01 |
| A9 | 0.0000E+00 | 0.0000E+00 | 0.0000E+00 | 0.0000E+00 |
| A10 | −4.9004E−02 | 2.7457E−01 | 1.3244E−01 | 2.0971E−02 |
| A11 | 0.0000E+00 | 0.0000E+00 | 0.0000E+00 | 0.0000E+00 |
| A12 | 9.5864E−02 | −2.9795E−01 | −2.0383E−01 | 3.2056E−01 |
| A13 | 0.0000E+00 | 0.0000E+00 | 0.0000E+00 | 0.0000E+00 |
| A14 | −9.2150E−02 | 2.0995E−01 | 2.1463E−01 | −4.9012E−01 |
| A15 | 0.0000E+00 | 0.0000E+00 | 0.0000E+00 | 0.0000E+00 |
| A16 | 4.8800E−02 | −9.2288E−02 | −1.3830E−01 | 3.3077E−01 |
| A17 | 0.0000E+00 | 0.0000E+00 | 0.0000E+00 | 0.0000E+00 |
| A18 | −1.3363E−02 | 2.2971E−02 | 4.7965E−02 | −1.1008E−01 |
| A19 | 0.0000E+00 | 0.0000E+00 | 0.0000E+00 | 0.0000E+00 |
| A20 | 1.4585E−03 | −2.4962E−03 | −6.9292E−03 | 1.4668E−02 |
| | 7 | 8 | 9 | 10 |
| R | −3.923 | −7.506 | −17.122 | 11.968 |
| K | 7.4201E+00 | −9.3224E+00 | 6.2324E+00 | 9.4626E+00 |
| A3 | 0.0000E+00 | 0.0000E+00 | −7.8210E−02 | −1.6253E−01 |
| A4 | 1.0834E−01 | 1.0546E−01 | 1.6359E−01 | 1.1451E−01 |
| A5 | 0.0000E+00 | 0.0000E+00 | −6.9974E−01 | 1.6252E−02 |
| A6 | 6.0569E−02 | 5.1050E−02 | 1.7567E+00 | −3.9107E−01 |
| A7 | 0.0000E+00 | 0.0000E+00 | −4.4098E+00 | 6.4928E−01 |
| A8 | −1.5180E−01 | −1.2772E−01 | 8.1853E+00 | −4.8473E−01 |
| A9 | 0.0000E+00 | 0.0000E+00 | −8.8961E+00 | −1.0082E−02 |
| A10 | 1.3577E−01 | 1.5293E−01 | 3.7169E+00 | 1.8644E−02 |
| A11 | 0.0000E+00 | 0.0000E+00 | 2.0390E+00 | 2.3821E−01 |
| A12 | −6.0842E−02 | −1.5333E−01 | −2.0215E+00 | 1.6126E−02 |
| A13 | 0.0000E+00 | 0.0000E+00 | −5.5851E−01 | −1.9467E−01 |
| A14 | 1.1372E−02 | 8.7307E−02 | 5.5971E−01 | −1.4557E−02 |
| A15 | 0.0000E+00 | 0.0000E+00 | 4.0324E−01 | 6.9647E−02 |
| A16 | 4.2270E−04 | −1.9267E−02 | −2.6487E−01 | 1.3562E−03 |
| A17 | 0.0000E+00 | 0.0000E+00 | −3.6966E−02 | 5.6057E−03 |
| A18 | 0.0000E+00 | 0.0000E+00 | 2.9742E−02 | −5.7985E−03 |
| A19 | 0.0000E+00 | 0.0000E+00 | 1.0596E−03 | −5.7629E−03 |
| A20 | 0.0000E+00 | 0.0000E+00 | −1.5750E−03 | 2.5602E−03 |

TABLE 33
Example 7

| | Si | | | |
|---|---|---|---|---|
| | 11 | 12 | 13 | 14 |
| R | −100.000 | −320.023 | 6.000 | 5.251 |
| K | 0.0000E+00 | 0.0000E+00 | 1.7568E+00 | 3.7949E+00 |
| A3 | −7.5631E−02 | 2.4723E−01 | 3.2739E−01 | 1.5352E−01 |
| A4 | −4.7215E−01 | −4.6254E−01 | −2.8675E−01 | −2.9873E−01 |
| A5 | 1.6614E+00 | 1.6254E−01 | −8.2020E−02 | 3.0495E−01 |
| A6 | −1.5901E+00 | 1.9632E−01 | 8.6748E−02 | −2.4741E−01 |
| A7 | 1.7894E−01 | −4.4351E−02 | 5.9729E−02 | 9.6922E−02 |
| A8 | 5.4492E−01 | −7.4234E−02 | −7.0621E−02 | −8.2224E−03 |
| A9 | −1.7923E−01 | −7.9104E−03 | 3.4700E−02 | −3.8649E−03 |
| A10 | −1.2075E−01 | 1.0506E−02 | −2.7538E−02 | 5.9829E−04 |
| A11 | 4.4137E−03 | 8.4351E−03 | −1.7687E−04 | −6.7697E−05 |
| A12 | 2.7472E−02 | 3.5087E−05 | 9.5013E−03 | −1.8700E−04 |
| A13 | 2.4770E−02 | −9.4139E−04 | 2.3210E−04 | 1.7233E−04 |
| A14 | 1.5813E−02 | −2.1972E−04 | −L0273E−03 | 9.0647E−06 |
| A15 | −1.8347E−02 | 4.0849E−05 | −1.8384E−04 | −1.8861E−05 |
| A16 | −L0390E−02 | −L0951E−04 | −4.6162E−05 | 1.8626E−06 |
| A17 | 3.7787E−04 | −7.0272E−05 | −3.3799E−05 | −3.7666E−07 |
| A18 | 2.0596E−03 | −L0607E−05 | 4.7828E−06 | 6.8013E−08 |
| A19 | 4.7212E−03 | 7.2571E−05 | 3.0460E−05 | −3.2817E−07 |
| A20 | −2.3000E−03 | −2.0034E−05 | −7.7035E−06 | 1.1796E−07 |

| | 15 | 16 |
|---|---|---|
| R | 1.854 | 1.708 |
| K | −6.1099E−01 | −1.0948E+00 |
| A3 | 3.9799E−02 | 4.7313E−02 |
| A4 | −4.4804E−01 | −3.2338E−01 |
| A5 | 1.8179E−01 | 1.7555E−01 |
| A6 | 3.4432E−02 | 1.6382E−02 |
| A7 | −3.7435E−04 | −4.1194E−02 |
| A8 | −1.0086E−02 | 7.8980E−03 |
| A9 | −1.4412E−03 | 1.2846E−03 |
| A10 | 7.4997E−04 | 1.2461E−04 |
| A11 | −1.7839E−04 | −6.8986E−05 |
| A12 | −8.3189E−05 | −5.9973E−05 |
| A13 | 6.9679E−05 | 4.4161E−06 |
| A14 | 4.3974E−05 | 1.3789E−06 |
| A15 | −5.4110E−06 | −1.9443E−06 |
| A16 | −2.7010E−06 | 4.6118E−07 |
| A17 | −6.8033E−07 | 3.4626E−07 |
| A18 | −2.0550E−07 | −3.0873E−08 |
| A19 | −9.8550E−08 | −3.0474E−08 |
| A20 | 7.2555E−08 | 4.0507E−09 |

TABLE 34
Example 7

| | |
|---|---|
| f | 4.27 |
| F-number | 1.6 |
| Total length | 5.598 |
| ω | 38.9 |

TABLE 35
Example 7

| | Focal length |
|---|---|
| L1 | 18.78 |
| L2 | 3.70 |
| L3 | −12.61 |
| L4 | −10.78 |
| L5 | −267.92 |
| L6 | −92.21 |
| L7 | 27.13 |

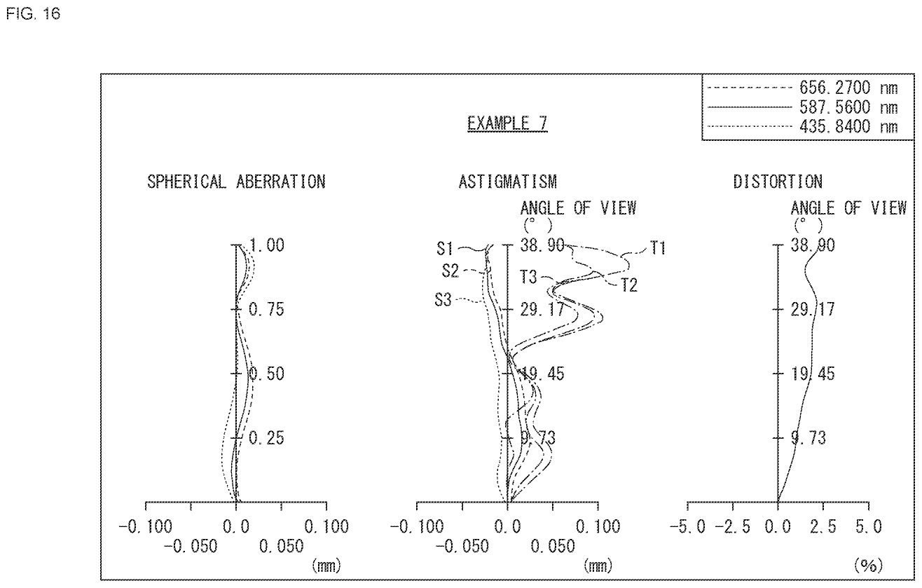

FIG. 16 describes various aberrations in Numerical example 7 described above.

As can be seen from each of the aberration diagrams, the imaging lens 7 according to Numerical example 7 has the various aberrations corrected optimally, while being small-sized with a great aperture. It is clear that the imaging lens 7 according to Numerical example 7 has superior image forming performance.

### Numerical Example 8

Table 36 describes basic lens data of Numerical example 8 in which specific numerical values are applied to the imaging lens 8 illustrated in FIG. 8. In the imaging lens 8 according to Numerical example 8, the seventh lens L7 has the negative refractive power in the vicinity of the optical axis.

In the imaging lens 8 according to Numerical example 8, both surfaces of each lens of the first lens L1 to the seventh lens L7 have aspheric shapes. Tables 37 and 38 describe values of coefficients representing the aspheric shapes.

Moreover, Table 39 describes values of a focal length f, an F-number, a total length, and a half angle of view ω of a whole lens system in the imaging lens 8 according to Numerical example 8. Table 40 describes values of a focal length of each of the first lens L1, the second lens L2, the third lens L3, the fourth lens L4, the fifth lens L5, the sixth lens L6, and the seventh lens L7.

TABLE 36
Example 8

| Si | Li | Ri | Di | Ndi | νdi |
|---|---|---|---|---|---|
| 1 | | | 0.180 | | |
| 2 | St | ∞ | −0.180 | | |
| 3 | L1R1 | 2.525 | 0.541 | 1.544 | 56.1 |
| 4 | L1R2 | 3.000 | 0.146 | | |
| 5 | L2R1 | 2.013 | 0.735 | 1.544 | 56.1 |
| 6 | L2R2 | −47.796 | 0.035 | | |
| 7 | L3R1 | −3.904 | 0.250 | 1.671 | 19.2 |
| 8 | L3R2 | −7.151 | 0.333 | | |
| 9 | L4R1 | −100.000 | 0.380 | 1.614 | 25.9 |
| 10 | L4R2 | −6661.139 | 0.208 | | |
| 11 | L5R1 | −100.000 | 0.360 | 1.544 | 56.1 |
| 12 | L5R2 | −320.023 | 0.111 | | |
| 13 | L6R1 | 6.000 | 0.493 | 1.614 | 25.9 |
| 14 | L6R2 | 5.251 | 0.247 | | |
| 15 | L7R1 | 1.756 | 0.522 | 1.535 | 55.7 |
| 16 | L7R2 | 1.099 | 0.640 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 37
Example 8

| | Si | | | |
|---|---|---|---|---|
| | 3 | 4 | 5 | 6 |
| R | 2.525 | 3.000 | 2.013 | −47.796 |
| K | −2.2679E−01 | −6.3668E+00 | −5.4988E+00 | 5.0700E+00 |
| A3 | 0.0000E+00 | 0.0000E+00 | 0.0000E+00 | 0.0000E+00 |
| A4 | −4.1694E−02 | −1.1058E−01 | −2.4014E−03 | −5.2669E−02 |
| A5 | 0.0000E+00 | 0.0000E+00 | 0.0000E+00 | 0.0000E+00 |
| A6 | 1.9094E−03 | 5.9097E−02 | −1.2310E−02 | 6.3270E−02 |
| A7 | 0.0000E+00 | 0.0000E+00 | 0.0000E+00 | 0.0000E+00 |
| A8 | −3.5400E−03 | −1.5324E−01 | −3.7501E−02 | −1.3404E−01 |
| A9 | 0.0000E+00 | 0.0000E+00 | 0.0000E+00 | 0.0000E+00 |
| A10 | −4.8378E−02 | 2.7433E−01 | 1.3256E−01 | 1.9894E−02 |
| A11 | 0.0000E+00 | 0.0000E+00 | 0.0000E+00 | 0.0000E+00 |
| A12 | 9.5366E−02 | −2.9904E−01 | −2.0402E−01 | 3.1954E−01 |
| A13 | 0.0000E+00 | 0.0000E+00 | 0.0000E+00 | 0.0000E+00 |
| A14 | −9.0955E−02 | 2.0979E−01 | 2.1368E−01 | −4.9255E−01 |
| A15 | 0.0000E+00 | 0.0000E+00 | 0.0000E+00 | 0.0000E+00 |
| A16 | 4.8600E−02 | −9.2169E−02 | −1.3831E−01 | 3.3269E−01 |
| A17 | 0.0000E+00 | 0.0000E+00 | 0.0000E+00 | 0.0000E+00 |
| A18 | −1.3725E−02 | 2.3029E−02 | 4.8076E−02 | −1.1011E−01 |
| A19 | 0.0000E+00 | 0.0000E+00 | 0.0000E+00 | 0.0000E+00 |
| A20 | 1.5915E−03 | −2.4983E−03 | −6.8690E−03 | 1.4511E−02 |
| | 7 | 8 | 9 | 10 |
| R | −3.904 | −7.151 | −100.000 | −6661.139 |
| K | 7.4201E+00 | −9.3224E+00 | 6.2324E+00 | 9.4626E+00 |
| A3 | 0.0000E+00 | 0.0000E+00 | −7.5490E−02 | −2.9114E−02 |
| A4 | 9.4779E−02 | 1.2441E−01 | 2.5168E−01 | 6.8039E−02 |
| A5 | 0.0000E+00 | 0.0000E+00 | −6.9277E−01 | 7.2374E−03 |
| A6 | 5.1965E−02 | 1.8857E−02 | 1.7357E+00 | −3.8940E−01 |
| A7 | 0.0000E+00 | 0.0000E+00 | −4.4297E+00 | 6.5398E−01 |
| A8 | −1.4892E−01 | −1.2729E−01 | 8.1770E+00 | −4.8543E−01 |
| A9 | 0.0000E+00 | 0.0000E+00 | −8.8961E+00 | −4.0552E−03 |
| A10 | 1.3709E−01 | 1.5610E−01 | 3.7183E+00 | 1.3731E−02 |
| A11 | 0.0000E+00 | 0.0000E+00 | 2.0409E+00 | 2.3511E−01 |
| A12 | −5.8816E−02 | −1.5018E−01 | −2.0203E+00 | 1.5250E−02 |
| A13 | 0.0000E+00 | 0.0000E+00 | −5.5788E−01 | −1.9447E−01 |
| A14 | 1.1294E−02 | 8.9072E−02 | 5.5799E−01 | −1.4035E−02 |
| A15 | 0.0000E+00 | 0.0000E+00 | 4.0594E−01 | 7.0248E−02 |
| A16 | −9.8407E−05 | −2.0406E−02 | −2.6492E−01 | 1.7077E−03 |
| A17 | 0.0000E+00 | 0.0000E+00 | −3.7454E−02 | 5.8389E−03 |
| A18 | 0.0000E+00 | 0.0000E+00 | 2.9421E−02 | −5.7504E−03 |
| A19 | 0.0000E+00 | 0.0000E+00 | 9.8689E−04 | −5.8380E−03 |
| A20 | 0.0000E+00 | 0.0000E+00 | −1.3238E−03 | 2.4676E−03 |

TABLE 38
Example 8

| | Si | | | |
|---|---|---|---|---|
| | 11 | 12 | 13 | 14 |
| R | −100.000 | −320.023 | 6.000 | 5.251 |
| K | 0.0000E+00 | 0.0000E+00 | 1.7568E+00 | 3.7949E+00 |
| A3 | 1.5804E−01 | 2.9293E−01 | 2.5434E−01 | 1.6084E−01 |
| A4 | −5.8688E−01 | −3.9622E−01 | −2.5944E−01 | −3.6405E−01 |
| A5 | 1.4352E+00 | 1.0853E−01 | −7.9828E−02 | 3.4806E−01 |
| A6 | −1.4630E+00 | 1.5702E−01 | 8.4182E−02 | −2.4632E−01 |
| A7 | 2.2329E−01 | −4.3049E−02 | 6.0171E−02 | 9.3360E−02 |
| A8 | 5.4826E−01 | −6.5390E−02 | −6.8321E−02 | −8.9638E−03 |
| A9 | −1.9340E−01 | −3.0304E−03 | 3.5668E−02 | −3.8306E−03 |
| A10 | −1.3293E−01 | 1.1753E−02 | −2.7536E−02 | 6.7770E−04 |
| A11 | −1.4272E−03 | 8.3015E−03 | −2.7743E−04 | −5.1218E−05 |
| A12 | 2.6321E−02 | −4.0688E−04 | 9.3639E−03 | −1.7726E−04 |
| A13 | 2.6799E−02 | −1.1746E−03 | 1.6098E−04 | 1.7499E−04 |
| A14 | 1.7283E−02 | −2.9880E−04 | −1.0559E−03 | 9.3528E−06 |
| A15 | −1.7222E−02 | −4.1280E−05 | −1.8918E−04 | −1.9118E−05 |
| A16 | −1.0143E−02 | −7.2297E−05 | −4.3274E−05 | 1.6887E−06 |
| A17 | 5.3877E−04 | −4.2223E−05 | −2.7956E−05 | −4.7346E−07 |
| A18 | 2.2667E−03 | −1.3251E−05 | 8.0183E−06 | 2.0338E−08 |
| A19 | 4.6020E−03 | 7.3875E−05 | 3.1615E−05 | −3.2629E−07 |
| A20 | −2.5701E−03 | −2.1501E−05 | −8.9355E−06 | 1.2711E−07 |

| | 15 | 16 |
|---|---|---|
| R | 1.756 | 1.099 |
| K | −6.1099E−01 | −1.0948E+00 |
| A3 | 2.8832E−02 | −1.2470E−01 |
| A4 | −4.9523E−01 | −2.9632E−01 |
| A5 | 1.8327E−01 | 1.9447E−01 |
| A6 | 3.7086E−02 | 1.1347E−02 |
| A7 | 4.2701E−04 | −4.2002E−02 |
| A8 | −9.0431E−03 | 8.1017E−03 |
| A9 | −1.3883E−03 | 1.3747E−03 |
| A10 | 7.1031E−04 | 1.1529E−04 |
| A11 | −1.9771E−04 | −7.9208E−05 |
| A12 | −8.9256E−05 | −6.2481E−05 |
| A13 | 6.8360E−05 | 4.8267E−06 |
| A14 | 4.3462E−05 | 2.1456E−06 |
| A15 | −5.5428E−06 | −1.9449E−06 |
| A16 | −2.7578E−06 | 4.5478E−07 |
| A17 | −7.0125E−07 | 3.4225E−07 |
| A18 | −2.0482E−07 | −3.2384E−08 |
| A19 | −9.4773E−08 | −3.0657E−08 |
| A20 | 7.6530E−08 | 4.2013E−09 |

TABLE 39
Example 8

| | |
|---|---|
| f | 4.25 |
| F-number | 1.6 |
| Total length | 5.312 |
| ω | 39.2 |

TABLE 40
Example 8

| | Focal length |
|---|---|
| L1 | 20.96 |
| L2 | 3.57 |
| L3 | −13.23 |
| L4 | −165.22 |
| L5 | −267.92 |
| L6 | −91.33 |
| L7 | −7.60 |

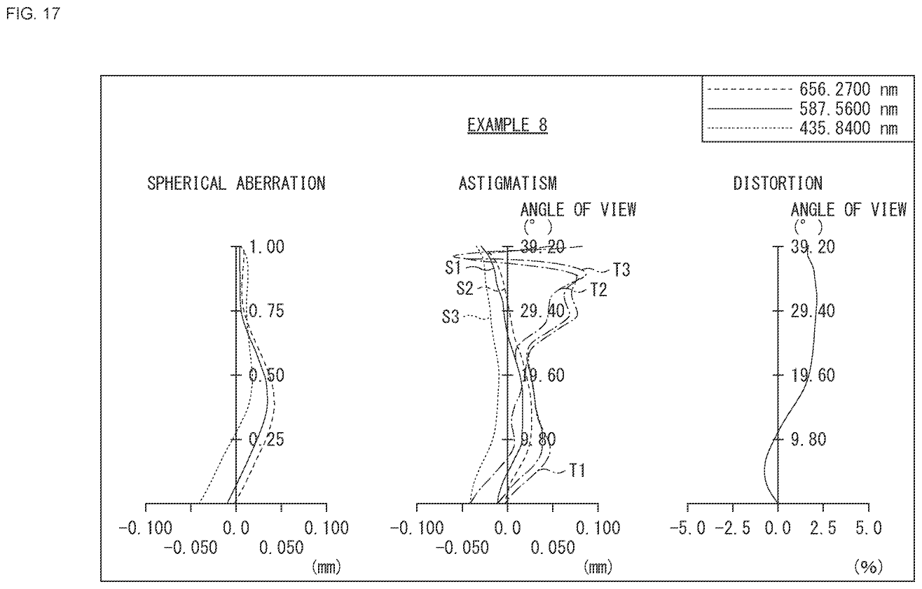

FIG. 17 describes various aberrations in Numerical example 8 described above.

As can be seen from each of the aberration diagrams, the imaging lens 8 according to Numerical example 8 has the various aberrations corrected optimally, while being small-sized with a great aperture. It is clear that the imaging lens 8 according to Numerical example 8 has superior image forming performance.

### Numerical Example 9

Table 41 describes basic lens data of Numerical example 9 in which specific numerical values are applied to the imaging lens 9 illustrated in FIG. 9. In the imaging lens 9 according to Numerical example 9, the seventh lens L7 has the positive refractive power in the vicinity of the optical axis.

In the imaging lens 9 according to Numerical example 9, both surfaces of each lens of the first lens L1 to the seventh lens L7 have aspheric shapes. Tables 42 and 43 describe values of coefficients representing the aspheric shapes.

Moreover, Table 44 describes values of a focal length f, an F-number, a total length, and a half angle of view ω of a whole lens system in the imaging lens 9 according to Numerical example 9. Table 45 describes values of a focal length of each of the first lens L1, the second lens L2, the third lens L3, the fourth lens L4, the fifth lens L5, the sixth lens L6, and the seventh lens L7.

TABLE 41
Example 9

| Si | Li | Ri | Di | Ndi | νdi |
|---|---|---|---|---|---|
| 1 | | | 0.180 | | |
| 2 | St | ∞ | −0.180 | | |
| 3 | L1R1 | 2.380 | 0.576 | 1.544 | 56.1 |
| 4 | L1R2 | 3.300 | 0.220 | | |
| 5 | L2R1 | 2.152 | 0.586 | 1.544 | 56.1 |
| 6 | L2R2 | −109.249 | 0.025 | | |
| 7 | L3R1 | −12.459 | 0.250 | 1.671 | 19.2 |
| 8 | L3R2 | 9.602 | 0.437 | | |
| 9 | L4R1 | −50.000 | 0.358 | 1.614 | 25.9 |
| 10 | L4R2 | −600.000 | 0.137 | | |
| 11 | L5R1 | −20.000 | 0.360 | 1.544 | 56.1 |
| 12 | L5R2 | −80.000 | 0.114 | | |
| 13 | L6R1 | 6.000 | 0.490 | 1.614 | 25.9 |
| 14 | L6R2 | 5.251 | 0.107 | | |
| 15 | L7R1 | 1.737 | 0.859 | 1.535 | 55.7 |
| 16 | L7R2 | 1.501 | 0.690 | | |
| 17 | SGR1 | ∞ | 0.110 | 1.517 | 64.2 |
| 18 | SGR2 | ∞ | 0.200 | | |
| 19 | IMG | ∞ | 0.000 | | |

TABLE 42
Example 9

| | Si | | | |
|---|---|---|---|---|
| | 3 | 4 | 5 | 6 |
| R | 2.380 | 3.300 | 2.152 | −109.249 |
| K | −2.2679E−01 | −6.3668E+00 | −5.4988E+00 | 5.0700E+00 |
| A3 | 0.0000E+00 | 0.0000E+00 | 0.0000E+00 | 0.0000E+00 |
| A4 | −3.1343E−02 | −7.6329E−02 | 4.2281E−03 | −1.6324E−02 |
| A5 | 0.0000E+00 | 0.0000E+00 | 0.0000E+00 | 0.0000E+00 |
| A6 | 1.3502E−02 | 4.7024E−02 | −3.4723E−02 | 6.1707E−02 |
| A7 | 0.0000E+00 | 0.0000E+00 | 0.0000E+00 | 0.0000E+00 |
| A8 | −1.9673E−02 | −1.5048E−01 | −1.5695E−02 | −1.5566E−01 |
| A9 | 0.0000E+00 | 0.0000E+00 | 0.0000E+00 | 0.0000E+00 |
| A10 | −3.3778E−02 | 2.7262E−01 | 1.1892E−01 | 1.5376E−02 |
| All | 0.0000E+00 | 0.0000E+00 | 0.0000E+00 | 0.0000E+00 |
| A12 | 9.3025E−02 | −3.0147E−01 | −2.0528E−01 | 3.2631E−01 |
| A13 | 0.0000E+00 | 0.0000E+00 | 0.0000E+00 | 0.0000E+00 |
| A14 | −9.4153E−02 | 2.1208E−01 | 2.1335E−01 | −4.8480E−01 |
| A15 | 0.0000E+00 | 0.0000E+00 | 0.0000E+00 | 0.0000E+00 |
| A16 | 4.8634E−02 | −9.2131E−02 | −1.3229E−01 | 3.2706E−01 |
| A17 | 0.0000E+00 | 0.0000E+00 | 0.0000E+00 | 0.0000E+00 |
| A18 | −1.2651E−02 | 2.2488E−02 | 4.4628E−02 | −1.0968E−01 |
| A19 | 0.0000E+00 | 0.0000E+00 | 0.0000E+00 | 0.0000E+00 |
| A20 | 1.3094E−03 | −2.3598E−03 | −6.3408E−03 | 1.4697E−02 |
| | 7 | 8 | 9 | 10 |
| R | −12.459 | 9.602 | −50.000 | −600.000 |
| K | 7.4201E+00 | −9.3224E+00 | 6.2324E+00 | 9.4626E+00 |
| A3 | 0.0000E+00 | 0.0000E+00 | −6.7318E−02 | 1.2797E−03 |
| A4 | 7.6417E−02 | 8.4486E−02 | 2.4622E−01 | −2.1729E−02 |
| A5 | 0.0000E+00 | 0.0000E+00 | −8.1159E−01 | 1.6353E−01 |
| A6 | 2.0252E−02 | 2.9382E−04 | 1.8604E+00 | −5.2473E−01 |
| A7 | 0.0000E+00 | 0.0000E+00 | −4.4999E+00 | 7.1737E−01 |
| A8 | −1.3060E−01 | −9.1830E−02 | 8.3450E+00 | −5.2304E−01 |
| A9 | 0.0000E+00 | 0.0000E+00 | −9.0282E+00 | −2.2399E−02 |
| A10 | 1.2857E−01 | 1.4051E−01 | 3.6728E+00 | 3.9304E−02 |
| A11 | 0.0000E+00 | 0.0000E+00 | 2.0342E+00 | 2.4843E−01 |
| A12 | −7.2177E−02 | −1.3906E−01 | −1.9182E+00 | 1.4653E−02 |
| A13 | 0.0000E+00 | 0.0000E+00 | −5.5926E−01 | −1.9956E−01 |
| A14 | 2.6470E−02 | 7.5030E−02 | 5.3655E−01 | −1.6578E−02 |
| A15 | 0.0000E+00 | 0.0000E+00 | 3.8686E−01 | 6.9542E−02 |
| A16 | −4.6670E−03 | −1.5807E−02 | −2.8132E−01 | 7.9609E−04 |
| A17 | 0.0000E+00 | 0.0000E+00 | −3.1586E−02 | 5.0706E−03 |
| A18 | 0.0000E+00 | 0.0000E+00 | 3.9883E−02 | −5.3959E−03 |
| A19 | 0.0000E+00 | 0.0000E+00 | 6.6891E−03 | −4.6015E−03 |
| A20 | 0.0000E+00 | 0.0000E+00 | −6.4064E−03 | 2.0663E−03 |

TABLE 43
Example 9

| | Si | | | |
|---|---|---|---|---|
| | 11 | 12 | 13 | 14 |
| R | −20.000 | −80.000 | 6.000 | 5.251 |
| K | 0.0000E+00 | 0.0000E+00 | 1.7568E+00 | 3.7949E+00 |
| A3 | 1.8258E−01 | 2.6397E−01 | 1.7123E−01 | 1.6945E−02 |
| A4 | −6.7356E−01 | −4.3375E−01 | −8.0638E−02 | −1.4567E−01 |
| A5 | 1.5971E+00 | 1.3087E−01 | −1.3075E−01 | 3.2264E−01 |
| A6 | −1.4746E+00 | 2.9494E−01 | 7.2337E−02 | −3.1308E−01 |
| A7 | 1.4763E−01 | −2.3837E−01 | 3.5418E−02 | 9.7890E−02 |
| A8 | 5.0260E−01 | 4.3325E−02 | −6.3323E−02 | 3.5986E−03 |
| A9 | −1.1666E−01 | −2.1660E−02 | 3.9572E−02 | −2.9129E−03 |
| A10 | −1.2141E−01 | 6.4198E−03 | −2.4605E−02 | −2.9398E−04 |
| A11 | −1.1639E−02 | 4.3652E−03 | 9.3217E−05 | −9.8995E−05 |
| A12 | 7.0993E−03 | 2.5511E−03 | 9.5560E−03 | −2.5929E−04 |
| A13 | 2.3778E−02 | −6.7492E−04 | −5.4535E−04 | 1.4485E−04 |
| A14 | 2.4499E−02 | −1.3661E−04 | −1.2088E−03 | 6.5175E−06 |
| A15 | −1.2994E−02 | −2.3740E−04 | −1.4744E−04 | −1.8820E−05 |
| A16 | −8.8941E−03 | −6.7487E−05 | −6.4920E−05 | −1.4416E−05 |
| A17 | −1.2051E−03 | −4.8301E−05 | −6.0587E−06 | 1.1191E−05 |
| A18 | 8.1243E−04 | −2.4913E−05 | 1.3049E−05 | −6.3515E−08 |
| A19 | 3.8419E−03 | 6.4685E−05 | 3.5896E−05 | −4.5384E−07 |
| A20 | −1.6060E−03 | −1.4490E−05 | −1.1489E−05 | −6.3689E−08 |

| | 15 | 16 |
|---|---|---|
| R | 1.737 | 1.501 |
| K | −6.1099E−01 | −1.0948E+00 |
| A3 | −6.3515E−02 | 3.9880E−02 |
| A4 | −2.9378E−01 | −3.5148E−01 |
| A5 | 1.0518E−01 | 2.0338E−01 |
| A6 | 4.2098E−02 | 7.7084E−03 |
| A7 | −3.0809E−03 | −4.6022E−02 |
| A8 | −8.8089E−03 | 1.1612E−02 |
| A9 | −1.1191E−03 | 2.0834E−03 |
| A10 | 8.3754E−04 | −8.0514E−04 |
| All | −2.0294E−04 | −3.2272E−05 |
| A12 | −8.9522E−05 | −1.4097E−05 |
| A13 | 7.0051E−05 | 3.0129E−06 |
| A14 | 4.0514E−05 | 2.4819E−06 |
| A15 | −5.3924E−06 | −1.5405E−06 |
| A16 | −2.7726E−06 | 2.5113E−07 |
| A17 | −5.9184E−07 | 3.3292E−07 |
| A18 | −1.7813E−07 | −4.8126E−08 |
| A19 | −8.1567E−08 | −3.2708E−08 |
| A20 | 6.4239E−08 | 6.1777E−09 |

TABLE 44
Example 9

| | |
|---|---|
| f | 4.13 |
| F-number | 1.4 |
| Total length | 5.520 |
| Ω | 39.3 |

TABLE 45
Example 9

| | Focal length |
|---|---|
| L1 | 12.88 |
| L2 | 3.89 |
| L3 | −8.05 |
| L4 | −88.79 |
| L5 | −49.20 |
| L6 | −91.15 |
| L7 | 77.41 |

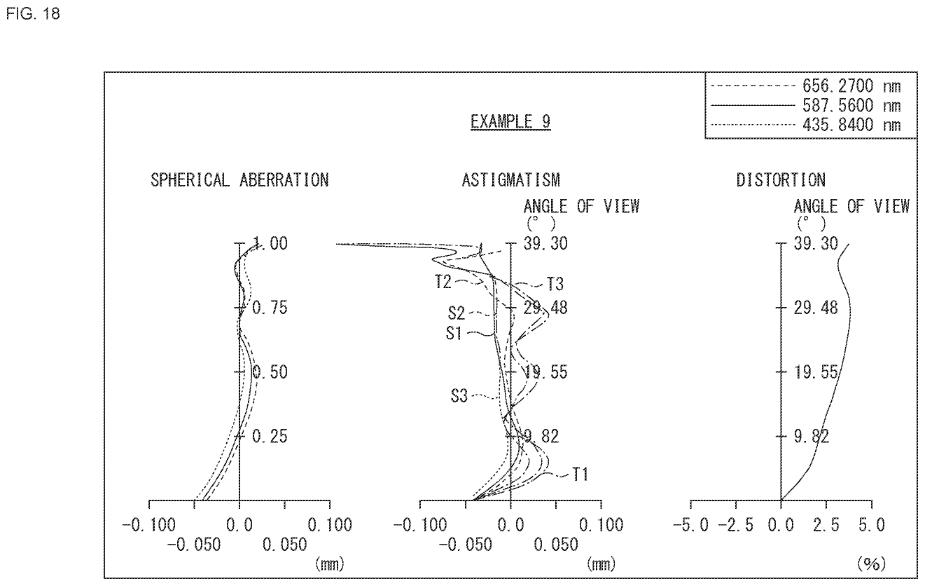

FIG. 18 describes various aberrations in Numerical example 9 described above.

As can be seen from each of the aberration diagrams, the imaging lens 9 according to Numerical example 9 has the various aberrations corrected optimally, while being small-sized with a great aperture. It is clear that the imaging lens 9 according to Numerical example 9 has superior image forming performance.

[Other Numerical Data regarding Each Example]

Tables 46 to 49 summarize values related to each of the forgoing conditional expressions for each numerical example. As can be seen from Tables 46 to 49, a value of each numerical example for each conditional expression falls within the range of the corresponding numerical value.

TABLE 46

| Conditional Expression | | Example 1 | Example 2 | Example 3 | Example 4 | Example 5 |
|---|---|---|---|---|---|---|
| (1) | TTL/f12 | 1.54 | 1.59 | 1.53 | 1.33 | 1.42 |
| (2) | f1/f | 1.72 | 248.31 | 3.84 | 3.89 | 0.89 |
| (3) | f2/f | 1.35 | 0.71 | 0.94 | 0.80 | 106.09 |
| (4) | νd(L3) | 19.2 | 19.2 | 19.2 | 25.9 | 19.2 |
| (5) | \|f3/f12\| | 2.70 | 2.62 | 4.60 | 1.55 | 3.21 |
| (6) | f3/f | −2.17 | −2.04 | −3.78 | −1.66 | −2.81 |
| (7) | f3/f456 | 0.31 | 0.32 | 1.36 | 0.46 | 0.24 |
| (8) | f3/f4567 | 0.62 | 0.63 | 1.76 | 0.39 | 0.61 |
| (9) | f4/f | −427.66 | −391.93 | −21.63 | −8.54 | −77.41 |
| (10) | \|f7/f12\| | 10.12 | 10.11 | 26.17 | 9.61 | 9.41 |
| (11) | \| f1/f34567 \| | 1.55 | 239.88 | 3.12 | 3.89 | 0.61 |
| (12) | \| f2/f34567 \| | 1.22 | 0.68 | 0.77 | 0.80 | 72.52 |
| (13) | f3/f4 | 0.0051 | 0.0052 | 0.1750 | 0.1949 | 0.0364 |
| (14) | f3/f5 | 0.29 | 0.27 | 0.91 | 0.17 | 0.17 |
| (15) | f45/f | −7.52 | −7.43 | −3.43 | −4.54 | −13.82 |

TABLE 47

| Conditional Expression | | Example 6 | Example 7 | Example 8 | Example 9 |
|---|---|---|---|---|---|
| (1) | TTL/f12 | 1.62 | 1.33 | 1.29 | 1.19 |
| (2) | f1/f | 27.77 | 4.40 | 4.93 | 3.12 |
| (3) | f2/f | 0.71 | 0.87 | 0.84 | 0.94 |
| (4) | νd(L3) | 19.2 | 19.2 | 19.2 | 19.2 |
| (5) | \|f3/f12\| | 1.94 | 2.99 | 3.20 | 1.73 |
| (6) | f3/f | −1.49 | −2.96 | −3.12 | −1.95 |
| (7) | f3/f456 | 0.04 | 1.40 | 0.28 | 0.35 |
| (8) | f3/f4567 | 0.06 | 1.21 | 2.04 | 0.32 |
| (9) | f4/f | −106.15 | −2.53 | −38.91 | −21.49 |
| (10) | \|f7/f12\| | 249.32 | 6.43 | 1.84 | 16.65 |
| (11) | \| f1/f34567 \| | 24.74 | 3.77 | 5.60 | 2.53 |
| (12) | \| f2/f34567 \| | 0.63 | 0.74 | 0.95 | 0.77 |
| (13) | f3/f4 | 0.0140 | 1.1697 | 0.0801 | 0.0907 |
| (14) | f3/f5 | 0.01 | 0.05 | 0.05 | 0.16 |
| (15) | f45/f | −52.84 | −2.43 | −24.05 | −7.65 |

TABLE 48

| | Example 1 | Example 2 | Example 3 | Example 4 | Example 5 |
|---|---|---|---|---|---|
| f | 4.69 | 4.69 | 4.64 | 4.21 | 4.71 |
| f1 | 8.06 | 1164.00 | 17.80 | 16.40 | 4.18 |
| f2 | 6.32 | 3.32 | 4.37 | 3.37 | 500.00 |
| f3 | −10.19 | −9.54 | −17.55 | −7.01 | −13.27 |
| f4 | −2004.60 | −1837.21 | −100.30 | −35.98 | −364.84 |
| f5 | −35.61 | −35.65 | −19.26 | −41.10 | −78.49 |
| f6 | −1811.81 | −302.26 | −100.54 | −85.74 | −500.00 |
| f7 | −38.27 | −36.88 | −99.83 | 43.58 | −38.88 |
| f12 | 3.78 | 3.65 | 3.82 | 4.54 | 4.13 |
| f45 | −35.27 | −34.83 | −15.92 | −19.13 | −65.13 |
| f456 | −32.41 | −29.52 | −12.89 | −15.13 | −54.74 |
| f4567 | −16.42 | −15.17 | −9.97 | −17.82 | −21.89 |
| f34567 | −5.20 | −4.85 | −5.70 | −4.21 | −6.90 |
| νd(L3) | 19.2 | 19.2 | 19.2 | 25.9 | 19.2 |
| TTL | 5.82 | 5.79 | 5.82 | 6.01 | 5.85 |

TABLE 49

| | Example 6 | Example 7 | Example 8 | Example 9 |
|---|---|---|---|---|
| f | 4.71 | 4.27 | 4.25 | 4.13 |
| f1 | 130.82 | 18.78 | 20.96 | 12.88 |
| f2 | 3.34 | 3.70 | 3.57 | 3.89 |
| f3 | −7.00 | −12.61 | −13.23 | −8.05 |
| f4 | −500.00 | −10.78 | −165.22 | −88.79 |
| f5 | −500.00 | −267.92 | −267.92 | −49.20 |
| f6 | −500.00 | −92.21 | −91.33 | −91.15 |
| f7 | −897.59 | 27.13 | −7.60 | 77.41 |
| f12 | 3.60 | 4.22 | 4.13 | 4.65 |
| f45 | −248.87 | −10.36 | −102.12 | −31.59 |
| f456 | −161.04 | −8.97 | −47.25 | −22.76 |
| f4567 | −120.83 | −10.38 | −6.49 | −25.14 |
| f34567 | −5.29 | −4.97 | −3.74 | −5.08 |
| νd(L3) | 19.2 | 19.2 | 19.2 | 19.2 |
| TTL | 5.85 | 5.60 | 5.31 | 5.52 |

### 5. APPLIED EXAMPLES

### [5.1 First Applied Example]

The technology according to the present disclosure is applicable to various products. For example, the technology according to the present disclosure may be implemented as an apparatus to be mounted on a movable body of any kind of an automobile, an electric automobile, a hybrid electric automobile, a motorcycle, a bicycle, a personal mobility, an airplane, a drone, a vessel, a robot, a construction machine, an agricultural machine (a tractor), and the like.

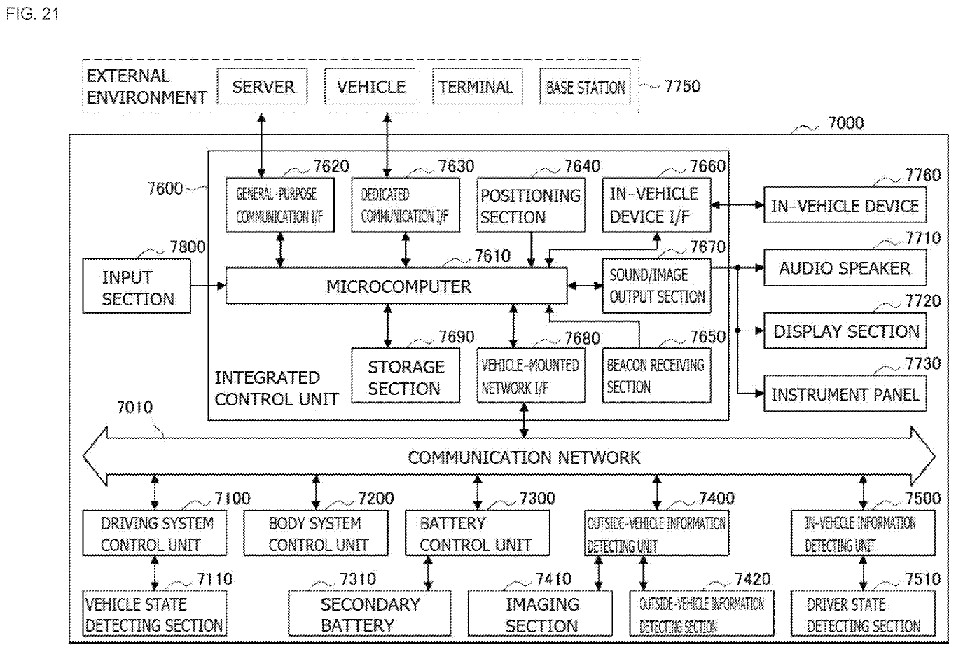

FIG. 21 is a block diagram depicting an example of schematic configuration of a vehicle control system 7000 as an example of a mobile body control system to which the technology according to an embodiment of the present disclosure can be applied. The vehicle control system 7000 includes a plurality of electronic control units connected to each other via a communication network 7010. In the example depicted in FIG. 21, the vehicle control system 7000 includes a driving system control unit 7100, a body system control unit 7200, a battery control unit 7300, an outside-vehicle information detecting unit 7400, an in-vehicle information detecting unit 7500, and an integrated control unit 7600. The communication network 7010 connecting the plurality of control units to each other may, for example, be a vehicle-mounted communication network compliant with an arbitrary standard such as controller area network (CAN), local interconnect network (LIN), local area network (LAN), FlexRay (registered trademark), or the like.

Each of the control units includes: a microcomputer that performs arithmetic processing according to various kinds of programs; a storage section that stores the programs executed by the microcomputer, parameters used for various kinds of operations, or the like; and a driving circuit that drives various kinds of control target devices. Each of the control units further includes: a network interface (I/F) for performing communication with other control units via the communication network 7010; and a communication I/F for performing communication with a device, a sensor, or the like within and without the vehicle by wire communication or radio communication. A functional configuration of the integrated control unit 7600 illustrated in FIG. 21 includes a microcomputer 7610, a general-purpose communication I/F 7620, a dedicated communication I/F 7630, a positioning section 7640, a beacon receiving section 7650, an in-vehicle device I/F 7660, a sound/image output section 7670, a vehicle-mounted network I/F 7680, and a storage section 7690. The other control units similarly include a microcomputer, a communication I/F, a storage section, and the like.

The driving system control unit 7100 controls the operation of devices related to the driving system of the vehicle in accordance with various kinds of programs. For example, the driving system control unit 7100 functions as a control device for a driving force generating device for generating the driving force of the vehicle, such as an internal combustion engine, a driving motor, or the like, a driving force transmitting mechanism for transmitting the driving force to wheels, a steering mechanism for adjusting the steering angle of the vehicle, a braking device for generating the braking force of the vehicle, and the like. The driving system control unit 7100 may have a function as a control device of an antilock brake system (ABS), electronic stability control (ESC), or the like.

The driving system control unit 7100 is connected with a vehicle state detecting section 7110. The vehicle state detecting section 7110, for example, includes at least one of a gyro sensor that detects the angular velocity of axial rotational movement of a vehicle body, an acceleration sensor that detects the acceleration of the vehicle, and sensors for detecting an amount of operation of an accelerator pedal, an amount of operation of a brake pedal, the steering angle of a steering wheel, an engine speed or the rotational speed of wheels, and the like. The driving system control unit 7100 performs arithmetic processing using a signal input from the vehicle state detecting section 7110, and controls the internal combustion engine, the driving motor, an electric power steering device, the brake device, and the like.

The body system control unit 7200 controls the operation of various kinds of devices provided to the vehicle body in accordance with various kinds of programs. For example, the body system control unit 7200 functions as a control device for a keyless entry system, a smart key system, a power window device, or various kinds of lamps such as a headlamp, a backup lamp, a brake lamp, a turn signal, a fog lamp, or the like. In this case, radio waves transmitted from a mobile device as an alternative to a key or signals of various kinds of switches can be input to the body system control unit 7200. The body system control unit 7200 receives these input radio waves or signals, and controls a door lock device, the power window device, the lamps, or the like of the vehicle.

The battery control unit 7300 controls a secondary battery 7310, which is a power supply source for the driving motor, in accordance with various kinds of programs. For example, the battery control unit 7300 is supplied with information about a battery temperature, a battery output voltage, an amount of charge remaining in the battery, or the like from a battery device including the secondary battery 7310. The battery control unit 7300 performs arithmetic processing using these signals, and performs control for regulating the temperature of the secondary battery 7310 or controls a cooling device provided to the battery device or the like.

The outside-vehicle information detecting unit 7400 detects information about the outside of the vehicle including the vehicle control system 7000. For example, the outside-vehicle information detecting unit 7400 is connected with at least one of an imaging section 7410 and an outside-vehicle information detecting section 7420. The imaging section 7410 includes at least one of a time-of-flight (ToF) camera, a stereo camera, a monocular camera, an infrared camera, and other cameras. The outside-vehicle information detecting section 7420, for example, includes at least one of an environmental sensor for detecting current atmospheric conditions or weather conditions and a peripheral information detecting sensor for detecting another vehicle, an obstacle, a pedestrian, or the like on the periphery of the vehicle including the vehicle control system 7000.

The environmental sensor, for example, may be at least one of a rain drop sensor detecting rain, a fog sensor detecting a fog, a sunshine sensor detecting a degree of sunshine, and a snow sensor detecting a snowfall. The peripheral information detecting sensor may be at least one of an ultrasonic sensor, a radar device, and a LIDAR device (Light detection and Ranging device, or Laser imaging detection and ranging device). Each of the imaging section 7410 and the outside-vehicle information detecting section 7420 may be provided as an independent sensor or device, or may be provided as a device in which a plurality of sensors or devices are integrated.

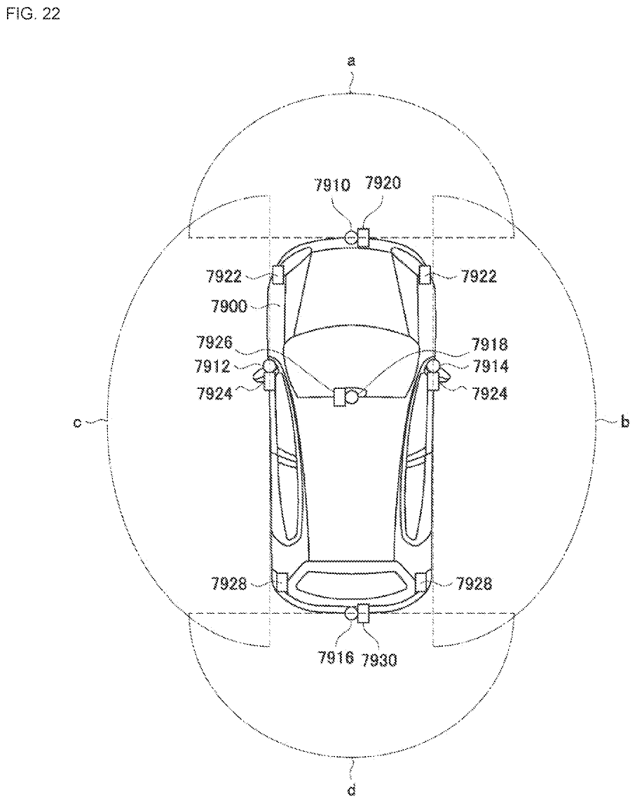

FIG. 22 depicts an example of installation positions of the imaging section 7410 and the outside-vehicle information detecting section 7420. Imaging sections 7910, 7912, 7914, 7916, and 7918 are, for example, disposed at at least one of positions on a front nose, sideview mirrors, a rear bumper, and a back door of the vehicle 7900 and a position on an upper portion of a windshield within the interior of the vehicle. The imaging section 7910 provided to the front nose and the imaging section 7918 provided to the upper portion of the windshield within the interior of the vehicle obtain mainly an image of the front of the vehicle 7900. The imaging sections 7912 and 7914 provided to the sideview mirrors obtain mainly an image of the sides of the vehicle 7900. The imaging section 7916 provided to the rear bumper or the back door obtains mainly an image of the rear of the vehicle 7900. The imaging section 7918 provided to the upper portion of the windshield within the interior of the vehicle is used mainly to detect a preceding vehicle, a pedestrian, an obstacle, a signal, a traffic sign, a lane, or the like.

Incidentally, FIG. 22 depicts an example of photographing ranges of the respective imaging sections 7910, 7912, 7914, and 7916. An imaging range a represents the imaging range of the imaging section 7910 provided to the front nose. Imaging ranges b and c respectively represent the imaging ranges of the imaging sections 7912 and 7914 provided to the sideview mirrors. An imaging range d represents the imaging range of the imaging section 7916 provided to the rear bumper or the back door. A bird's-eye image of the vehicle 7900 as viewed from above can be obtained by superimposing image data imaged by the imaging sections 7910, 7912, 7914, and 7916, for example.

Outside-vehicle information detecting sections 7920, 7922, 7924, 7926, 7928, and 7930 provided to the front, rear, sides, and corners of the vehicle 7900 and the upper portion of the windshield within the interior of the vehicle may be, for example, an ultrasonic sensor or a radar device. The outside-vehicle information detecting sections 7920, 7926, and 7930 provided to the front nose of the vehicle 7900, the rear bumper, the back door of the vehicle 7900, and the upper portion of the windshield within the interior of the vehicle may be a LIDAR device, for example. These outside-vehicle information detecting sections 7920 to 7930 are used mainly to detect a preceding vehicle, a pedestrian, an obstacle, or the like.

Returning to FIG. 21, the description will be continued. The outside-vehicle information detecting unit 7400 makes the imaging section 7410 image an image of the outside of the vehicle, and receives imaged image data. In addition, the outside-vehicle information detecting unit 7400 receives detection information from the outside-vehicle information detecting section 7420 connected to the outside-vehicle information detecting unit 7400. In a case where the outside-vehicle information detecting section 7420 is an ultrasonic sensor, a radar device, or a LIDAR device, the outside-vehicle information detecting unit 7400 transmits an ultrasonic wave, an electromagnetic wave, or the like, and receives information of a received reflected wave. On the basis of the received information, the outside-vehicle information detecting unit 7400 may perform processing of detecting an object such as a human, a vehicle, an obstacle, a sign, a character on a road surface, or the like, or processing of detecting a distance thereto. The outside-vehicle information detecting unit 7400 may perform environment recognition processing of recognizing a rainfall, a fog, road surface conditions, or the like on the basis of the received information. The outside-vehicle information detecting unit 7400 may calculate a distance to an object outside the vehicle on the basis of the received information.

In addition, on the basis of the received image data, the outside-vehicle information detecting unit 7400 may perform image recognition processing of recognizing a human, a vehicle, an obstacle, a sign, a character on a road surface, or the like, or processing of detecting a distance thereto. The outside-vehicle information detecting unit 7400 may subject the received image data to processing such as distortion correction, alignment, or the like, and combine the image data imaged by a plurality of different imaging sections 7410 to generate a bird's-eye image or a panoramic image. The outside-vehicle information detecting unit 7400 may perform viewpoint conversion processing using the image data imaged by the imaging section 7410 including the different imaging parts.

The in-vehicle information detecting unit 7500 detects information about the inside of the vehicle. The in-vehicle information detecting unit 7500 is, for example, connected with a driver state detecting section 7510 that detects the state of a driver. The driver state detecting section 7510 may include a camera that images the driver, a biosensor that detects biological information of the driver, a microphone that collects sound within the interior of the vehicle, or the like. The biosensor is, for example, disposed in a seat surface, the steering wheel, or the like, and detects biological information of an occupant sitting in a seat or the driver holding the steering wheel. On the basis of detection information input from the driver state detecting section 7510, the in-vehicle information detecting unit 7500 may calculate a degree of fatigue of the driver or a degree of concentration of the driver, or may determine whether the driver is dozing. The in-vehicle information detecting unit 7500 may subject an audio signal obtained by the collection of the sound to processing such as noise canceling processing or the like.

The integrated control unit 7600 controls general operation within the vehicle control system 7000 in accordance with various kinds of programs. The integrated control unit 7600 is connected with an input section 7800. The input section 7800 is implemented by a device capable of input operation by an occupant, such, for example, as a touch panel, a button, a microphone, a switch, a lever, or the like. The integrated control unit 7600 may be supplied with data obtained by voice recognition of voice input through the microphone. The input section 7800 may, for example, be a remote control device using infrared rays or other radio waves, or an external connecting device such as a mobile telephone, a personal digital assistant (PDA), or the like that supports operation of the vehicle control system 7000. The input section 7800 may be, for example, a camera. In that case, an occupant can input information by gesture. Alternatively, data may be input which is obtained by detecting the movement of a wearable device that an occupant wears. Further, the input section 7800 may, for example, include an input control circuit or the like that generates an input signal on the basis of information input by an occupant or the like using the above-described input section 7800, and which outputs the generated input signal to the integrated control unit 7600. An occupant or the like inputs various kinds of data or gives an instruction for processing operation to the vehicle control system 7000 by operating the input section 7800.

The storage section 7690 may include a read only memory (ROM) that stores various kinds of programs executed by the microcomputer and a random access memory (RAM) that stores various kinds of parameters, operation results, sensor values, or the like. In addition, the storage section 7690 may be implemented by a magnetic storage device such as a hard disc drive (HDD) or the like, a semiconductor storage device, an optical storage device, a magneto-optical storage device, or the like.

The general-purpose communication I/F 7620 is a communication I/F used widely, which communication I/F mediates communication with various apparatuses present in an external environment 7750. The general-purpose communication I/F 7620 may implement a cellular communication protocol such as global system for mobile communications (GSM (registered trademark)), worldwide interoperability for microwave access (WiMAX (registered trademark)), long term evolution (LTE (registered trademark)), LTE-advanced (LTE-A), or the like, or another wireless communication protocol such as wireless LAN (referred to also as wireless fidelity (Wi-Fi (registered trademark)), Bluetooth (registered trademark), or the like. The general-purpose communication I/F 7620 may, for example, connect to an apparatus (for example, an application server or a control server) present on an external network (for example, the Internet, a cloud network, or a company-specific network) via a base station or an access point. In addition, the general-purpose communication I/F 7620 may connect to a terminal present in the vicinity of the vehicle (which terminal is, for example, a terminal of the driver, a pedestrian, or a store, or a machine type communication (MTC) terminal) using a peer to peer (P2P) technology, for example.

The dedicated communication I/F 7630 is a communication I/F that supports a communication protocol developed for use in vehicles. The dedicated communication I/F 7630 may implement a standard protocol such, for example, as wireless access in vehicle environment (WAVE), which is a combination of institute of electrical and electronic engineers (IEEE) 802.11p as a lower layer and IEEE 1609 as a higher layer, dedicated short range communications (DSRC), or a cellular communication protocol. The dedicated communication I/F 7630 typically carries out V2X communication as a concept including one or more of communication between a vehicle and a vehicle (Vehicle to Vehicle), communication between a road and a vehicle (Vehicle to Infrastructure), communication between a vehicle and a home (Vehicle to Home), and communication between a pedestrian and a vehicle (Vehicle to Pedestrian).

The positioning section 7640, for example, performs positioning by receiving a global navigation satellite system (GNSS) signal from a GNSS satellite (for example, a GPS signal from a global positioning system (GPS) satellite), and generates positional information including the latitude, longitude, and altitude of the vehicle. Incidentally, the positioning section 7640 may identify a current position by exchanging signals with a wireless access point, or may obtain the positional information from a terminal such as a mobile telephone, a personal handyphone system (PHS), or a smart phone that has a positioning function.

The beacon receiving section 7650, for example, receives a radio wave or an electromagnetic wave transmitted from a radio station installed on a road or the like, and thereby obtains information about the current position, congestion, a closed road, a necessary time, or the like. Incidentally, the function of the beacon receiving section 7650 may be included in the dedicated communication I/F 7630 described above.

The in-vehicle device I/F 7660 is a communication interface that mediates connection between the microcomputer 7610 and various in-vehicle devices 7760 present within the vehicle. The in-vehicle device I/F 7660 may establish wireless connection using a wireless communication protocol such as wireless LAN, Bluetooth (registered trademark), near field communication (NFC), or wireless universal serial bus (WUSB). In addition, the in-vehicle device I/F 7660 may establish wired connection by universal serial bus (USB), high-definition multimedia interface (HDMI (registered trademark)), mobile high-definition link (MHL), or the like via a connection terminal (and a cable if necessary) not depicted in the figures. The in-vehicle devices 7760 may, for example, include at least one of a mobile device and a wearable device possessed by an occupant and an information device carried into or attached to the vehicle. The in-vehicle devices 7760 may also include a navigation device that searches for a path to an arbitrary destination. The in-vehicle device I/F 7660 exchanges control signals or data signals with these in-vehicle devices 7760.

The vehicle-mounted network I/F 7680 is an interface that mediates communication between the microcomputer 7610 and the communication network 7010. The vehicle-mounted network I/F 7680 transmits and receives signals or the like in conformity with a predetermined protocol supported by the communication network 7010.

The microcomputer 7610 of the integrated control unit 7600 controls the vehicle control system 7000 in accordance with various kinds of programs on the basis of information obtained via at least one of the general-purpose communication I/F 7620, the dedicated communication I/F 7630, the positioning section 7640, the beacon receiving section 7650, the in-vehicle device I/F 7660, and the vehicle-mounted network I/F 7680. For example, the microcomputer 7610 may calculate a control target value for the driving force generating device, the steering mechanism, or the braking device on the basis of the obtained information about the inside and outside of the vehicle, and output a control command to the driving system control unit 7100. For example, the microcomputer 7610 may perform cooperative control intended to implement functions of an advanced driver assistance system (ADAS) which functions include collision avoidance or shock mitigation for the vehicle, following driving based on a following distance, vehicle speed maintaining driving, a warning of collision of the vehicle, a warning of deviation of the vehicle from a lane, or the like. In addition, the microcomputer 7610 may perform cooperative control intended for automatic driving, which makes the vehicle to travel autonomously without depending on the operation of the driver, or the like, by controlling the driving force generating device, the steering mechanism, the braking device, or the like on the basis of the obtained information about the surroundings of the vehicle.

The microcomputer 7610 may generate three-dimensional distance information between the vehicle and an object such as a surrounding structure, a person, or the like, and generate local map information including information about the surroundings of the current position of the vehicle, on the basis of information obtained via at least one of the general-purpose communication I/F 7620, the dedicated communication I/F 7630, the positioning section 7640, the beacon receiving section 7650, the in-vehicle device I/F 7660, and the vehicle-mounted network I/F 7680. In addition, the microcomputer 7610 may predict danger such as collision of the vehicle, approaching of a pedestrian or the like, an entry to a closed road, or the like on the basis of the obtained information, and generate a warning signal. The warning signal may, for example, be a signal for producing a warning sound or lighting a warning lamp.

The sound/image output section 7670 transmits an output signal of at least one of a sound and an image to an output device capable of visually or auditorily notifying information to an occupant of the vehicle or the outside of the vehicle. In the example of FIG. 21, an audio speaker 7710, a display section 7720, and an instrument panel 7730 are illustrated as the output device. The display section 7720 may, for example, include at least one of an on-board display and a head-up display. The display section 7720 may have an augmented reality (AR) display function. The output device may be other than these devices, and may be another device such as headphones, a wearable device such as an eyeglass type display worn by an occupant or the like, a projector, a lamp, or the like. In a case where the output device is a display device, the display device visually displays results obtained by various kinds of processing performed by the microcomputer 7610 or information received from another control unit in various forms such as text, an image, a table, a graph, or the like. In addition, in a case where the output device is an audio output device, the audio output device converts an audio signal constituted of reproduced audio data or sound data or the like into an analog signal, and auditorily outputs the analog signal.

Incidentally, at least two control units connected to each other via the communication network 7010 in the example depicted in FIG. 21 may be integrated into one control unit. Alternatively, each individual control unit may include a plurality of control units. Further, the vehicle control system 7000 may include another control unit not depicted in the figures. In addition, part or the whole of the functions performed by one of the control units in the above description may be assigned to another control unit. That is, predetermined arithmetic processing may be performed by any of the control units as long as information is transmitted and received via the communication network 7010. Similarly, a sensor or a device connected to one of the control units may be connected to another control unit, and a plurality of control units may mutually transmit and receive detection information via the communication network 7010.

In the vehicle control system 7000 described above, the imaging lens and the imaging apparatus of the present disclosure are applicable to any of the imaging section 7410 and the imaging sections 7910, 7912, 7914, 7916, and 7918.

### [5.2 Second Applied Example]

The technology according to the present disclosure may be applied to an endoscopic surgery system.

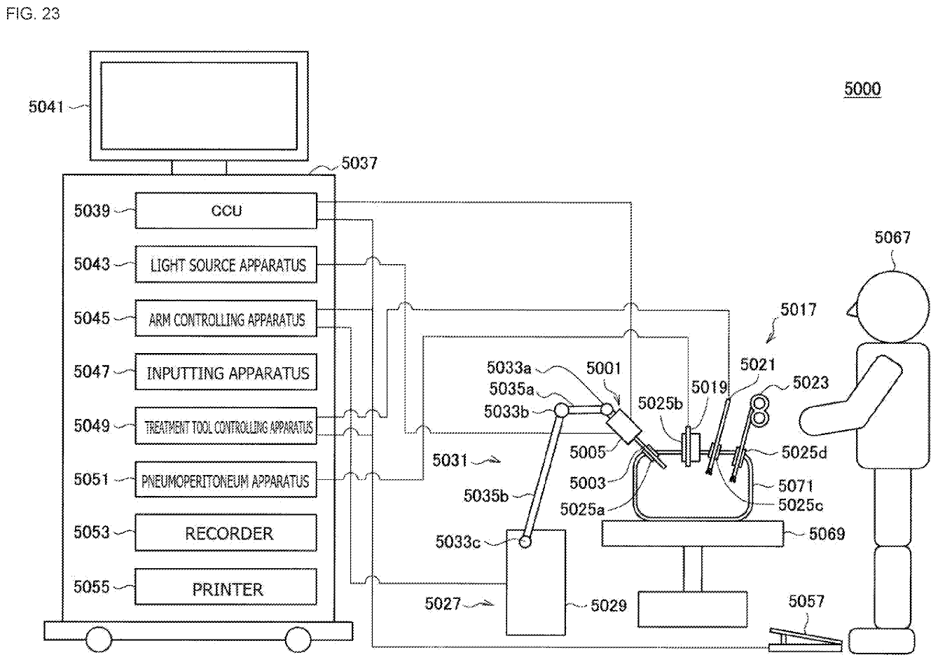

FIG. 23 is a view depicting an example of a schematic configuration of an endoscopic surgery system 5000 to which the technology according to an embodiment of the present disclosure can be applied. In FIG. 23, a state is illustrated in which a surgeon (medical doctor) 5067 is using the endoscopic surgery system 5000 to perform surgery for a patient 5071 on a patient bed 5069. As depicted, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body cavity of the patient 5071 through the trocars 5025a to 5025d. In the example depicted, as the other surgical tools 5017, a pneumoperitoneum tube 5019, an energy device 5021 and forceps 5023 are inserted into body cavity of the patient 5071. Further, the energy device 5021 is a treatment tool for performing incision and peeling of a tissue, sealing of a blood vessel or the like by high frequency current or ultrasonic vibration. However, the surgical tools 5017 depicted are mere examples at all, and as the surgical tools 5017, various surgical tools which are generally used in endoscopic surgery such as, for example, tweezers or a retractor may be used.

An image of a surgical region in a body cavity of the patient 5071 imaged by the endoscope 5001 is displayed on a display apparatus 5041. The surgeon 5067 would use the energy device 5021 or the forceps 5023 while watching the image of the surgical region displayed on the display apparatus 5041 on the real time basis to perform such treatment as, for example, resection of an affected area. It is to be noted that, though not depicted, the pneumoperitoneum tube 5019, the energy device 5021 and the forceps 5023 are supported by the surgeon 5067, an assistant or the like during surgery.

(Supporting Arm Apparatus)

The supporting arm apparatus 5027 includes an arm unit 5031 extending from a base unit 5029. In the example depicted, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endo scope 5001 can be implemented.

(Endoscope)

The endoscope 5001 includes the lens barrel 5003 which has a region of a predetermined length from a distal end thereof to be inserted into a body cavity of the patient 5071, and a camera head 5005 connected to a proximal end of the lens barrel 5003. In the example depicted, the endoscope 5001 is depicted as a rigid endoscope having the lens barrel 5003 of the hard type. However, the endoscope 5001 may otherwise be configured as a flexible endoscope having the lens barrel 5003 of the flexible type.

The lens barrel 5003 has, at a distal end thereof, an opening in which an objective lens is fitted. A light source apparatus 5043 is connected to the endoscope 5001 such that light generated by the light source apparatus 5043 is introduced to a distal end of the lens barrel by a light guide extending in the inside of the lens barrel 5003 and is irradiated toward an observation target in a body cavity of the patient 5071 through the objective lens. It is to be noted that the endoscope 5001 may be a forward-viewing endoscope or may be an oblique-viewing endoscope or a side-viewing endoscope.

An optical system and an image pickup element are provided in the inside of the camera head 5005 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image pickup elements may be provided on the camera head 5005. In this case, a plurality of relay optical systems are provided in the inside of the lens barrel 5003 in order to guide observation light to each of the plurality of image pickup elements.

(Various Apparatus Incorporated in Cart)

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process). The CCU 5039 provides the image signal for which the image processes have been performed to the display apparatus 5041. Further, the CCU 5039 transmits a control signal to the camera head 5005 to control driving of the camera head 5005. The control signal may include information relating to an image pickup condition such as a magnification or a focal distance.

The display apparatus 5041 displays an image based on an image signal for which the image processes have been performed by the CCU 5039 under the control of the CCU 5039. If the endo scope 5001 is ready for imaging of a high resolution such as 4K (horizontal pixel number 3840×vertical pixel number 216), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical region to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined controlling method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image pickup condition (type of irradiation light, magnification, focal distance or the like) by the endoscope 5001, an instruction to drive the energy device 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video imaged by the camera. Further, the inputting apparatus 5047 includes a microphone which can collect the voice of a user, and various kinds of inputting are performed by voice collected by the microphone. By configuring the inputting apparatus 5047 such that various kinds of information can be inputted in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy device 5021 for cautery or incision of a tissue, sealing of a blood vessel or the like. A pneumoperitoneum apparatus 5051 feeds gas into a body cavity of the patient 5071 through the pneumoperitoneum tube 5019 to inflate the body cavity in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

(Supporting Arm Apparatus)

The supporting arm apparatus 5027 includes the base unit 5029 serving as a base, and the arm unit 5031 extending from the base unit 5029. In the example depicted, the arm unit 5031 includes the plurality of joint portions 5033a, 5033b and 5033c and the plurality of links 5035a and 5035b connected to each other by the joint portion 5033b. In FIG. 23, for simplified illustration, the configuration of the arm unit 5031 is depicted in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body cavity of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the rotational angle of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known controlling methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the operating room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches with and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical region can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperate with each other to implement driving control of the arm unit 5031.

(Light Source Apparatus)

The light source apparatus 5043 supplies irradiation light upon imaging of a surgical region to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 5043. Further, in this case, if laser beams from the respective RGB laser light sources are irradiated time-divisionally on an observation target and driving of the image pickup elements of the camera head 5005 is controlled in synchronism with the irradiation timings, then images individually corresponding to the R, G and B colors can be picked up time-divisionally. According to the method just described, a color image can be obtained even if a color filter is not provided for the image pickup element.

Further, driving of the light source apparatus 5043 may be controlled such that the intensity of light to be outputted is changed for each predetermined time. By controlling driving of the image pickup element of the camera head 5005 in synchronism with the timing of the change of the intensity of light to acquire images time-divisionally and synthesizing the images, an image of a high dynamic range free from underexposed blocked up shadows and overexposed highlights can be created.

Further, the light source apparatus 5043 may be configured to supply light of a predetermined wavelength band ready for special light observation. In special light observation, for example, by utilizing the wavelength dependency of absorption of light in a body tissue to irradiate light of a narrower wavelength band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5043 can be configured to supply such narrow-band light and/or excitation light suitable for special light observation as described above.

(Camera Head and CCU)

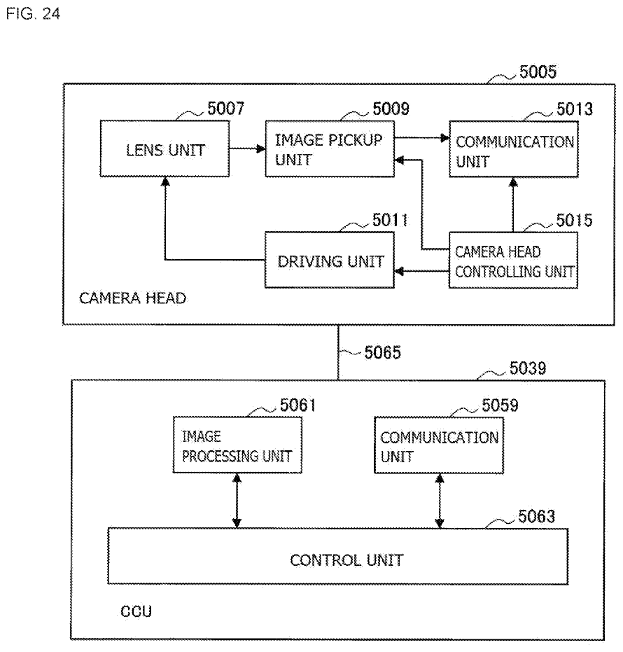

Functions of the camera head 5005 of the endoscope 5001 and the CCU 5039 are described in more detail with reference to FIG. 24. FIG. 24 is a block diagram depicting an example of a functional configuration of the camera head 5005 and the CCU 5039 depicted in FIG. 23.

Referring to FIG. 24, the camera head 5005 has, as functions thereof, a lens unit 5007, an image pickup unit 5009, a driving unit 5011, a communication unit 5013 and a camera head controlling unit 5015. Further, the CCU 5039 has, as functions thereof, a communication unit 5059, an image processing unit 5061 and a control unit 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from a distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image pickup element of the image pickup unit 5009. Further, the zoom lens and the focusing lens are configured such that the positions thereof on their optical axis are movable for adjustment of the magnification and the focal point of a picked up image.

The image pickup unit 5009 includes an image pickup element and disposed at a succeeding stage to the lens unit 5007. Observation light having passed through the lens unit 5007 is condensed on the light receiving face of the image pickup element, and an image signal corresponding to the observation image is generated by photoelectric conversion of the image pickup element. The image signal generated by the image pickup unit 5009 is provided to the communication unit 5013.

As the image pickup element which is included by the image pickup unit 5009, an image sensor, for example, of the complementary metal oxide semiconductor (CMOS) type is used which has a Bayer array and is capable of picking up an image in color. It is to be noted that, as the image pickup element, an image pickup element may be used which is ready, for example, for imaging of an image of a high resolution equal to or not less than 4K. If an image of a surgical region is obtained in a high resolution, then the surgeon 5067 can comprehend a state of the surgical region in enhanced details and can proceed with the surgery more smoothly.

Further, the image pickup element which is included by the image pickup unit 5009 includes such that it has a pair of image pickup elements for acquiring image signals for the right eye and the left eye compatible with 3D display. Where 3D display is applied, the surgeon 5067 can comprehend the depth of a living body tissue in the surgical region more accurately. It is to be noted that, if the image pickup unit 5009 is configured as that of the multi-plate type, then a plurality of systems of lens units 5007 are provided corresponding to the individual image pickup elements of the image pickup unit 5009.

The image pickup unit 5009 may not necessarily be provided on the camera head 5005. For example, the image pickup unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controlling unit 5015. Consequently, the magnification and the focal point of a picked up image by the image pickup unit 5009 can be adjusted suitably.

The communication unit 5013 includes a communication apparatus for transmitting and receiving various kinds of information to and from the CCU 5039. The communication unit 5013 transmits an image signal acquired from the image pickup unit 5009 as RAW data to the CCU 5039 through the transmission cable 5065. Thereupon, in order to display a picked up image of a surgical region in low latency, preferably the image signal is transmitted by optical communication. This is because, upon surgery, the surgeon 5067 performs surgery while observing the state of an affected area through a picked up image, it is demanded for a moving image of the surgical region to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image pickup conditions such as, for example, information that a frame rate of a picked up image is designated, information that an exposure value upon image picking up is designated and/or information that a magnification and a focal point of a picked up image are designated. The communication unit 5013 provides the received control signal to the camera head controlling unit 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controlling unit 5015.

It is to be noted that the image pickup conditions such as the frame rate, exposure value, magnification or focal point are set automatically by the control unit 5063 of the CCU 5039 on the basis of an acquired image signal. In other words, an auto exposure (AE) function, an auto focus (AF) function and an auto white balance (AWB) function are incorporated in the endoscope 5001.

The camera head controlling unit 5015 controls driving of the camera head 5005 on the basis of a control signal from the CCU 5039 received through the communication unit 5013. For example, the camera head controlling unit 5015 controls driving of the image pickup element of the image pickup unit 5009 on the basis of information that a frame rate of a picked up image is designated and/or information that an exposure value upon image picking up is designated. Further, for example, the camera head controlling unit 5015 controls the driving unit 5011 to suitably move the zoom lens and the focus lens of the lens unit 5007 on the basis of information that a magnification and a focal point of a picked up image are designated. The camera head controlling unit 5015 may further include a function for storing information for identifying the lens barrel 5003 and/or the camera head 5005.

It is to be noted that, by disposing the components such as the lens unit 5007 and the image pickup unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processing unit 5061.

Further, the communication unit 5059 transmits, to the camera head 5005, a control signal for controlling driving of the camera head 5005. The control signal may also be transmitted by optical communication.

The image processing unit 5061 performs various image processes for an image signal in the form of RAW data transmitted thereto from the camera head 5005. The image processes include various known signal processes such as, for example, a development process, an image quality improving process (a bandwidth enhancement process, a super-resolution process, a noise reduction (NR) process and/or an image stabilization process) and/or an enlargement process (electronic zooming process). Further, the image processing unit 5061 performs a detection process for an image signal in order to perform AE, AF and AWB.

The image processing unit 5061 includes a processor such as a CPU or a GPU, and when the processor operates in accordance with a predetermined program, the image processes and the detection process described above can be performed. It is to be noted that, where the image processing unit 5061 includes a plurality of GPUs, the image processing unit 5061 suitably divides information relating to an image signal such that image processes are performed in parallel by the plurality of GPUs.

The control unit 5063 performs various kinds of control relating to image picking up of a surgical region by the endoscope 5001 and display of the picked up image. For example, the control unit 5063 generates a control signal for controlling driving of the camera head 5005. Thereupon, if image pickup conditions are inputted by the user, then the control unit 5063 generates a control signal on the basis of the input by the user. Alternatively, where the endoscope 5001 has an AE function, an AF function and an AWB function incorporated therein, the control unit 5063 suitably calculates an optimum exposure value, focal distance and white balance in response to a result of a detection process by the image processing unit 5061 and generates a control signal.

Further, the control unit 5063 controls the display apparatus 5041 to display an image of a surgical region on the basis of an image signal for which image processes have been performed by the image processing unit 5061. Thereupon, the control unit 5063 recognizes various objects in the surgical region image using various image recognition technologies. For example, the control unit 5063 can recognize a surgical tool such as forceps, a particular living body region, bleeding, mist when the energy device 5021 is used and so forth by detecting the shape, color and so forth of edges of the objects included in the surgical region image. The control unit 5063 causes, when it controls the display unit 5041 to display a surgical region image, various kinds of surgery supporting information to be displayed in an overlapping manner with an image of the surgical region using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fiber ready for optical communication or a composite cable ready for both of electrical and optical communication.

Here, while, in the example depicted, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the operating room. Therefore, such a situation that movement of medical staff in the operating room is disturbed by the transmission cable 5065 can be eliminated.

An example of the endoscopic surgery system 5000 to which the technology according to an embodiment of the present disclosure can be applied has been described above. It is to be noted here that, although the endoscopic surgery system 5000 has been described as an example, the system to which the technology according to an embodiment of the present disclosure can be applied is not limited to the example. For example, the technology according to an embodiment of the present disclosure may be applied to a flexible endoscopic system for inspection or a microscopic surgery system.

The technology according to the present disclosure is suitably applicable to the camera head 5005. In particular, the imaging lens of the present disclosure is suitably applicable to the lens unit 5007 of the camera head 5005.

### 6. OTHER EMBODIMENTS

The technology according to the present disclosure is not limited to the forgoing description of the embodiment and the working examples, and may be carried out in various modifications.

For example, the shapes and the numerical values of the respective parts described in the forgoing numerical examples are each a mere example of the implementation of the present technology, and the technical scope of the present technology should not be construed as being limited by these examples.

Further, in the embodiments and the working examples above, the configuration including substantially seven lenses has been described; however, a configuration may be provided that further includes a lens substantially having no refractive power.

Further, for example, the present technology may be provided with the following configurations.

According to the present technology in the following configurations, with a seven-lens overall configuration, optimization of a configuration of each lens is attained. Hence, it is possible to provide a high-performance imaging lens or an imaging apparatus that attain size reduction and an increase in aperture size.
[1]

An imaging lens including, in order from object side toward image plane side:

a first lens having positive refractive power in the vicinity of an optical axis;

a second lens having positive refractive power in the vicinity of the optical axis;

a third lens having negative refractive power in the vicinity of the optical axis;

a fourth lens having negative refractive power in the vicinity of the optical axis;

a fifth lens having negative refractive power in the vicinity of the optical axis;

a sixth lens having negative refractive power in the vicinity of the optical axis; and

a seventh lens of which a lens surface on the image plane side has an aspheric shape having an inflection point.
[2]

The imaging lens according to [1] described above, in which the following conditional expression is satisfied,

1.1<TTL/f12<1.8 (1)

where “TTL” is a distance along the optical axis from a vertex of a surface on the object side of the first lens to an image plane, and “f12” is a composite focal length of the first lens and the second lens.
[3]

The imaging lens according to [1] described above, in which the following conditional expression is satisfied,

0.8<f1/f <273.0 (2)

where “f1” is a focal length of the first lens, and “f” is a focal length of a whole lens system.
[4]

The imaging lens according to [1] described above, in which the following conditional expression is satisfied,

0.6<f2/f<116.0 (3)

where “f2” is a focal length of the second lens, and “f” is a focal length of a whole lens system.
[5]

The imaging lens according to [1] described above, in which the following conditional expression is satisfied,

17.3<vd(L3)<28.5 (4)

where “vd(L3)” is an Abbe number of the third lens with respect to a d-line.
[6]

The imaging lens according to [1] described above, in which the following conditional expression is satisfied,

1.4<|f3/f12|<5.1 (5)

where “f3” is a focal length of the third lens, and “f12” is a composite focal length of the first lens and the second lens.
[7]

The imaging lens according to [1] described above, in which the following conditional expression is satisfied,

−4.2<f3/f<−1.3 (6)

where “f3” is a focal length of the third lens, and “f” is a focal length of a whole lens system.
[8]

The imaging lens according to [1] described above, in which the following conditional expression is satisfied,

0.0<f3/f456<1.5 (7)

where “f3” is a focal length of the third lens, and “f456” is a composite focal length of the fourth lens, the fifth lens, and the sixth lens.
[9]

The imaging lens according to [1] described above, in which the following conditional expression is satisfied,

0.0<f3/f4567<2.2 (8)

where “f3” is a focal length of the third lens, and “f4567” is a composite focal length of the fourth lens, the fifth lens, the sixth lens, and the seventh lens.
[10]

The imaging lens according to [1] described above, in which the following conditional expression is satisfied,

−470.0<f4/f<−2.3 (9)

where “f4” is a focal length of the fourth lens, and “f” is a focal length of a whole lens system.
[11]

The imaging lens according to [1] described above, in which the following conditional expression is satisfied,

1.7<|f7/f12|<274.0 (10)

where “f7” is a focal length of the seventh lens, and “f12” is a composite focal length of the first lens and the second lens.
[12]

The imaging lens according to [1] described above, in which the following conditional expression is satisfied,

0.5<|f1/f34567|<263.0 (11)

where “f1” is a focal length of the first lens, and “f34567” is a composite focal length of the third lens, the fourth lens, the fifth lens, the sixth lens, and the seventh lens.
[13]

The imaging lens according to [1] described above, in which the following conditional expression is satisfied,

0.6<|f2/f34567|<79.8 (12)

where “f2” is a focal length of the second lens, and “f34567” is a composite focal length of the third lens, the fourth lens, the fifth lens, the sixth lens, and the seventh lens.
[14]

The imaging lens according to [1] described above, in which the following conditional expression is satisfied,

0.0<f3/f4<1.3 (13)

where “f3” is a focal length of the third lens, and “f4” is a focal length of the fourth lens.
[15]

The imaging lens according to [1] described above, in which the following conditional expression is satisfied,

0.0<f3/f5<1.0 (14)

where “f3” is a focal length of the third lens, and “f5” is a focal length of the fifth lens.
[16]

The imaging lens according to [1] described above, in which the following conditional expression is satisfied,

−58.1<f45/f<−2.2 (15)

where “f45” is a composite focal length of the fourth lens and the fifth lens, and “f” is a focal length of a whole lens system.
[17]

The imaging lens according to [1] described above, in which an aperture stop is disposed between a lens surface on the object side of the first lens and a lens surface on the image plane side of the first lens.
[18]

An imaging apparatus including an imaging lens and an imaging element that outputs an imaging signal corresponding to an optical image formed by the imaging lens, the imaging lens including, in order from object side toward image plane side:

a first lens having positive refractive power in the vicinity of an optical axis;

a second lens having positive refractive power in the vicinity of the optical axis;

a third lens having negative refractive power in the vicinity of the optical axis;

a fourth lens having negative refractive power in the vicinity of the optical axis;

a fifth lens having negative refractive power in the vicinity of the optical axis;

a sixth lens having negative refractive power in the vicinity of the optical axis; and

a seventh lens of which a lens surface on the image plane side has an aspheric shape having an inflection point.
[19]

The imaging lens according to any one of [1] to [17] described above, further including a lens that substantially has no refractive power.
[20]

The imaging apparatus according to [18] described above, in which the imaging lens further includes a lens that substantially has no refractive power.

This application claims the priority on the basis of Japanese Patent Application No. 2018-203211 filed with Japan Patent Office on Oct. 29, 2018, the entire contents of which are incorporated in this application by reference.

It should be understood by those skilled in the art that various modifications, combinations, sub-combinations, and alterations may occur depending on design requirements and other factors insofar as they are within the scope of the appended claims or the equivalents thereof.

1. An imaging lens comprising, in order from object side toward image plane side:
a first lens having positive refractive power in vicinity of an optical axis;
a second lens having positive refractive power in the vicinity of the optical axis;
a third lens having negative refractive power in the vicinity of the optical axis;
a fourth lens having negative refractive power in the vicinity of the optical axis;
a fifth lens having negative refractive power in the vicinity of the optical axis;
a sixth lens having negative refractive power in the vicinity of the optical axis; and
a seventh lens of which a lens surface on the image plane side has an aspheric shape having an inflection point.

2. The imaging lens according to claim 1, wherein the following conditional expression is satisfied,
1.1<i TTL/f12<1.8 (1)
where “TTL” is a distance along the optical axis from a vertex of a surface on the object side of the first lens to an image plane, and “f12” is a composite focal length of the first lens and the second lens.

3. The imaging lens according to claim 1, wherein the following conditional expression is satisfied,
0.8<f1/f<273.0 (2)
where “f1” is a focal length of the first lens, and “f” is a focal length of a whole lens system.

4. The imaging lens according to claim 1, wherein the following conditional expression is satisfied,
0.6<f2/f<116.0 (3)
where “f2” is a focal length of the second lens, and “f” is a focal length of a whole lens system.

5. The imaging lens according to claim 1, wherein the following conditional expression is satisfied,
17.3<vd(L3)<28.5 (4)
where “vd(L3)” is an Abbe number of the third lens with respect to a d-line.

6. The imaging lens according to claim 1, wherein the following conditional expression is satisfied,
1.4<|f3/f12|<5.1 (5)
where “f3” is a focal length of the third lens, and “f12” is a composite focal length of the first lens and the second lens.

7. The imaging lens according to claim 1, wherein the following conditional expression is satisfied,
−4.2<f3/f<−1.3 (6)
where “f3” is a focal length of the third lens, and “f” is a focal length of a whole lens system.

8. The imaging lens according to claim 1, wherein the following conditional expression is satisfied,
0.0<f3/f456<1.5 (7)
where “f3” is a focal length of the third lens, and “f456” is a composite focal length of the fourth lens, the fifth lens, and the sixth lens.

9. The imaging lens according to claim 1, wherein the following conditional expression is satisfied,
0.0<f3/f4567<2.2 (8)
where “f3” is a focal length of the third lens, and “f4567” is a composite focal length of the fourth lens, the fifth lens, the sixth lens, and the seventh lens.

10. The imaging lens according to claim 1, wherein the following conditional expression is satisfied,
−470.0<f4/f<−2.3 (9)
where “f4” is a focal length of the fourth lens, and “f” is a focal length of a whole lens system.

11. The imaging lens according to claim 1, wherein the following conditional expression is satisfied,
1.7<|f7/f12|<274.0 (10)
where “f7” is a focal length of the seventh lens, and “f12” is a composite focal length of the first lens and the second lens.

12. The imaging lens according to claim 1, wherein the following conditional expression is satisfied,
0.5<|f1/f34567|<263.0 (11)
where “f1” is a focal length of the first lens, and “f34567” is a composite focal length of the third lens, the fourth lens, the fifth lens, the sixth lens, and the seventh lens.

13. The imaging lens according to claim 1, wherein the following conditional expression is satisfied,
0.6<|f2/f34567|<79.8 (12)
where “f2” is a focal length of the second lens, and “f34567” is a composite focal length of the third lens, the fourth lens, the fifth lens, the sixth lens, and the seventh lens.

14. The imaging lens according to claim 1, wherein the following conditional expression is satisfied,
0.0<f3/f4<1.3 (13)
where “f3” is a focal length of the third lens, and “f4” is a focal length of the fourth lens.

15. The imaging lens according to claim 1, wherein the following conditional expression is satisfied,
0.0<f3/f5<1.0 (14)
where “f3” is a focal length of the third lens, and “f5” is a focal length of the fifth lens.

16. The imaging lens according to claim 1, wherein the following conditional expression is satisfied,
−58.1<f45/f<−2.2 (15)
where “f45” is a composite focal length of the fourth lens and the fifth lens, and “f” is a focal length of a whole lens system.

17. The imaging lens according to claim 1, wherein an aperture stop is disposed between a lens surface on the object side of the first lens and a lens surface on the image plane side of the first lens.

18. An imaging apparatus including an imaging lens and an imaging element that outputs an imaging signal corresponding to an optical image formed by the imaging lens, the imaging lens comprising, in order from object side toward image plane side:
a first lens having positive refractive power in vicinity of an optical axis;
a second lens having positive refractive power in the vicinity of the optical axis;
a third lens having negative refractive power in the vicinity of the optical axis;
a fourth lens having negative refractive power in the vicinity of the optical axis;
a fifth lens having negative refractive power in the vicinity of the optical axis;
a sixth lens having negative refractive power in the vicinity of the optical axis; and
a seventh lens of which a lens surface on the image plane side has an aspheric shape having an inflection point.
